(19) United States
(12) Patent Application Publication
(10) Pub. No.: US 2026/0009027 A1
(43) Pub. Date: Jan. 8, 2026

(54) PRIME EDITING-MEDIATED READTHROUGH OF PREMATURE TERMINATION CODONS (PERT)

(71) Applicants: The Broad Institute, Inc., Cambridge, MA (US); President and Fellows of Harvard College, Cambridge, MA (US)

(72) Inventors: David R. Liu, Cambridge, MA (US); Aditya Raguram, Cambridge, MA (US); Steven Erwood, Cambridge, MA (US); Sarah Pierce, Cambridge, MA (US); Olukeyede Oye, Cambridge, MA (US)

(73) Assignees: The Broad Institute, Inc., Cambridge, MA (US); President and Fellows of Harvard College, Cambridge, MA (US)

(21) Appl. No.: 19/271,745

(22) Filed: Jul. 16, 2025

Related U.S. Application Data
(63) Continuation of application No. PCT/US2024/011892, filed on Jan. 17, 2024.
(60) Provisional application No. 63/480,495, filed on Jan. 18, 2023.
(60) Provisional application No. 63/579,778, filed on Aug. 30, 2023.

(51) Int. Cl. C12N 15/11; C12N 9/12; C12N 9/22

(52) U.S. Cl. CPC C12N 15/11; C12N 9/1252; C12N 9/1276; C12N 9/226; C12Y 207/07007; C12Y 207/07049; C07K 2319/00; C12N 2310/20

(57) ABSTRACT

Aspects of the disclosure relate to methods, compositions, and systems for editing an endogenous tRNA into a suppressor tRNA, or alternatively, replacing said endogenous tRNA with a suppressor tRNA with using prime editing. Additional aspects relate to compositions comprising the prime editing machinery, pegRNAs, and/or complexes comprising the prime editor and pegRNA that are capable of editing and/or replacing an endogenous tRNA to yield a suppressor tRNA. In some aspects, the disclosure further relates to polynucleotides encoding one or more nucleic acid sequences encoding the prime editor and/or pegRNA, cells comprising the polynucleotides and complexes comprising the prime editor and pegRNA, kits comprising any one of the compositions, complexes, polynucleotides, vectors, and/or cells disclosed herein, and/or delivery systems for administering any one of the compositions, complexes, polynucleotides, vectors to a subject in need thereof. Additional aspects relate to methods for inserting a new suppressor tRNA gene into a target site in a genome (e.g., a safe harbor locus site) using prime editing.

### RELATED APPLICATIONS

This application claims priority under 35 U.S.C. §§ 120 and 365(c) to International PCT Application, PCT/US2024/011892, filed Jan. 17, 2024, which claims priority under 35 U.S.C. § 119(e) to U.S. Provisional Application Ser. No. 63/480,495, filed Jan. 18, 2023, and U.S. Provisional Application Ser. No. 63/579,778, filed Aug. 30, 2023, each of which is incorporated herein by reference.

### GOVERNMENT SUPPORT

This invention was made with government support under R35GM118062 awarded by NIH MIRA. The government has certain rights in the invention.

### REFERENCE TO AN ELECTRONIC SEQUENCE LISTING

The contents of the electronic sequence listing (B119570177US02-SEQ-GJM.xml; Size: 40,985,063 bytes; and Date of Creation: Jul. 14, 2025) are herein incorporated by reference in its entirety.

### BACKGROUND

Nonsense mutations in genomic DNA lead to premature termination codons (PTCs) in mRNAs, which in turn impede translation of full-length proteins. Diminished translation of full-length proteins due to PTCs can induce pathogenic effects in cells and organisms. Indeed, approximately 33% of known human genetic diseases and 11% of known pathogenic gene variants are caused by PTCs. Interestingly, many bacteria and viruses utilize suppressor tRNAs to enable translational stop codon readthrough (e.g., the ribosome goes past the stop codon and continues translating the mRNA into protein). However, suppressor tRNAs do not naturally occur in humans. Suppressor tRNAs were recently used to rescue a genetic disease in a mouse model carrying a nonsense mutation. While the therapy was found safe in humans, the suppressor tRNA was delivered via an adeno-associated viral vector (herein “AAV”). It is generally known in the art that permanent expression of the suppressor tRNA is necessary for continued rescue from the disease, which is challenging to achieve using AAV and requires repeated administration of the suppressor tRNA vector. Prime editing allows for precise editing of the genomic DNA encoding tRNAs and may provide a platform for the treatment of diseases associated with PTCs.

### SUMMARY

Aspects of the present disclosure relate to compositions and methods for generating suppressor tRNAs from endogenous tRNAs, using prime editing, to enable readthrough of premature termination codons. In some embodiments, the methods and compositions relate to editing a DNA sequence encoding the anticodon of an endogenous tRNA to read through amber, ochre, or opal stop codons. In other embodiments, the methods and compositions relate to editing a DNA sequence to change the identity of the tRNA molecules charged amino acid. Combinations are also possible, for example, in some embodiments the DNA sequences encoding the anticodon and the charging loops are edited sequentially or simultaneously. Additional embodiments relate to compositions and methods for replacing a DNA sequence encoding an endogenous tRNA with a DNA sequence encoding a suppressor tRNA. Other embodiments still, relate to compositions and methods for inserting a DNA sequence encoding a suppressor tRNA gene into a target site of the genome, such as a safe harbor site. Other aspects of the disclosure further relate to methods for selecting suppressor tRNAs with high read-through efficiency of PTCs and methods of selecting pegRNAs to edit endogenous tRNA genes into suppressor tRNA genes.

Additional aspects disclosed herein relate to compositions comprising the prime editing machinery (e.g., fusion protein comprising a nucleic acid programmable DNA binding protein and reverse transcriptase and/or pegRNA, etc.) and/or complexes comprising the prime editor and pegRNA that are capable of editing an endogenous tRNA into a suppressor tRNA. In some aspects, the disclosure further relates to polynucleotides encoding one or more nucleic acid sequences encoding the prime editor and/or pegRNA, vectors encoding said polynucleotides, cells comprising the polynucleotides and complexes comprising the prime editor and pegRNA, kits comprising any one of the compositions, complexes, polynucleotides, vectors (e.g., AAV), and/or cells disclosed herein, and/or delivery systems for administering any one of the compositions, complexes, polynucleotides, vectors to a subject in need thereof (e.g., lipid nanoparticles). Other aspects relate to methods for inserting a new suppressor tRNA gene into a target site in a genome (e.g., a safe harbor locus site) using prime editing and methods for delivering the compositions, complexes, polynucleotides, and vectors using virus-like particles.

As defined elsewhere herein, suppressor tRNAs are endogenous tRNAs that are naturally charged with their cognate amino acids but possess engineered anticodon loops designed to bind PTCs (e.g., amber, ochre, or opal stop codons). As such, these suppressor tRNAs bind to PTCs during the process of translation, leading to incorporation of an amino acid instead of terminating translation.

Humans (and other species, such as domesticated animals and plants) possess over 500 interspersed tRNA genes, and many of these genes are redundant and dispensable (see Table 1). For example, many endogenous tRNA possess isodecorders, which as used herein, refers to tRNA molecules sharing the same anticodon sequence but diverging elsewhere in their sequence. These isodecoders are redundant and dispensable, and thus, can be edited using prime editing to convert endogenous tRNA molecules into suppressor tRNA molecules capable of reading through PTCs. As another example, there are multiple instances of non-synonymous anticodon mutations being found in healthy study participants, supporting the redundancy and dispensability of certain tRNA genes. For instance, one or both copies of the tRNA^{Lys} CUU gene is deleted in ˜50% of humans. As such, prime editing could be used to convert the CUU anticodon of this tRNALys gene into UUA, UCA, or CUA for ochre (e.g., 5′ UAA 3′), opal (e.g., 5′ UGA 3′), and amber (e.g., 5 UAG 3′) stop codon suppression, respectively (e.g., it would permit creation of a suppressor tRNALys from an endogenous tRNALys).

The inventors of the present disclosure have developed a technique known as “PERT” or prime editing-mediated readthrough of premature termination codons (herein “PERT”). PERT uses prime editing to convert a DNA sequence encoding an endogenous tRNA gene into a DNA sequence encoding a suppressor tRNA molecule with a bespoke anticodon loop designed to suppress PTCs (e.g., amber, ochre, or opal stop codons, see FIG. 1). The skilled artisan will appreciate that using the prime editing techniques disclosed herein permits permanent expression of the suppressor tRNA, leading to persistent PTC readthrough and rescue of protein expression. Without wishing to be bound by any particular theory, it is believed that because long-term expression of suppressor tRNAs is well tolerated in human cells and in mice. PERT may be used to safely rescue desired protein expression while otherwise minimally perturbing treated cells. Additionally, or alternatively, prime editing may be used to customize other parts of the tRNA beyond the anticodon loop to modulate amino acid identity and other tRNA characteristics in a subject in need thereof.

As described above, several aspects of the present relate to methods and compositions for generating suppressor tRNAs for readthrough of PTCs. One aspect, according to the present disclosure, relates to compositions and methods for editing a DNA sequence encoding the anticodon of an endogenous tRNA, using prime editing, such that the edited tRNA is capable of reading through an amber, ochre, or opal stop codon. For example, prime editing may be used to convert the tRNA^{Lys}-CUU (e.g., CULU is the anticodon) gene into tRNA^{Lys}-UUA. tRNA^{Lys}-UCA, or tRNA^{Lys}-CUA for ochre (e.g., 5′ UAA 3′), opal (e.g., 5′ UGA 3′), and amber (e.g., 5′ UAG 3′) suppression, respectively, to generate an endogenous suppressor tRNA^{Lys}.

In some embodiments, the methods and compositions comprise editing a DNA sequence encoding a tRNA at a target site. Any suitable tRNA gene known to the skilled artisan may be edited using the methods disclosed herein, such as those listed in Table 1. In some cases, the target site is an anticodon sequence located within an anticodon arm domain of the endogenous tRNA gene. However, other targets sites are also possible. For instance, the target site may be any domain, or combination of domains, of an endogenous tRNA gene. Non-limiting examples include a D-arm domain, a T-arm domain, a variable arm domain, or an acceptor stem domain.

In some embodiments, the methods and compositions comprise contacting the DNA sequence at the target site with a prime editor and a pegRNA. The prime editor, in some cases, installs one or more modifications at the target site, relative to an unedited endogenous tRNA gene, thus converting the tRNA gene into a suppressor tRNA gene. In some embodiments, the prime editor substitutes the DNA sequence of the endogenous tRNA gene encoding the anticodon sequence with a nonsense suppressor anticodon sequence. In some embodiments, the nonsense suppressor anticodon sequence is 5′-UUA-3′. In some embodiments, the nonsense suppressor anticodon sequence is 5′-UCA-3′. In some embodiments, the nonsense suppressor anticodon sequence is 5′-CUA-3′. In some embodiments, the nonsense suppressor anticodon 5′-UUA-3′ is configured to bind to an ochre premature termination codon (PTC) having the sequence 5′-UAA-3′. In some embodiments, the nonsense suppressor anticodon 5′-UCA-3′ is configured to bind to an opal premature termination codon (PTC) having the sequence 5′-UGA-3′. In some embodiments, the nonsense suppressor anticodon 5′-CUA-3′ is configured to bind to an amber premature termination codon (PTC) having the sequence 5′-UAG-3′.

In some embodiments, the prime editor installs one or more modifications at a target site different than the anticodon sequence within the anticodon arm domain of the endogenous tRNA. For example, in some cases, the prime editor installs a single base nucleotide in the variable arm domain of the tRNA. Editing the variable arm domain of tRNAs is known in the art to result in replacement of the cognate amino acid (e.g., alanine) with a non-cognate amino acid (e.g., serine). In some embodiments, the non-cognate amino acid is serine. In other cases, the prime editor installs one or more edits within the acceptor stem domain of the endogenous tRNA molecule. In some embodiments, the installing the one or more edits within the acceptor stem of the endogenous tRNA results in the replacement of the cognate amino acid with a non-cognate amino acid. For example, installation of a C70U mutation in the acceptor stem domain of the tRNA is known in the art to create a G3:U70 base pair in the acceptor stem domain, which facilitates replacement of the cognate amino acid with the non-cognate amino acid alanine.

In some embodiments, the choice of amino acid to be charged to the suppressor tRNA is tailored by the choice of tRNA to edit and/or by installing sequences recognized by specific aminoacyl-tRNA synthetases to direct amino acid charging of the newly generated suppressor tRNA. In some embodiments, suppression with widely tolerated amino acids, such as glycine, alanine, or serine, may be preferable to suppression with more unusual amino acids, such as proline, arginine, or tryptophan, except when treating diseases caused by premature stop codons that have arisen from mutation of these amino acids. For example, in certain embodiments, mutations of arginine codons (e.g., 5′-CGA-3′ codons mutated to 5′-UGA-3′) to STOP codons are a common cause of genetic diseases, and in these cases, prime editing can be used to create an arginine-charged suppressor tRNA may be especially desirable. This may be accomplished, for example, by prime-editing the anticodon of an arginine charged tRNA to an anticodon that recognizes the TGA stop codon (corresponding to a TCA anticodon).

Without wishing to be bound by any particular theory, it is possible that repurposing certain endogenous tRNAs into suppressor tRNAs may negatively perturb cell function. Alternatively, or additionally, it is possible that the ideal target tRNA candidate for conversion to a suppressor tRNA may not be expressed in a target tissue, and thus would likely elicit poor levels of readthrough. As such, certain embodiments relate to overwriting an existing highly expressed RNA sequence, using prime editing, with the desired suppressor tRNA. In some embodiments, any RNA gene may be overwritten with the sequence of an optimized suppressor tRNA candidate sequence. The DNA sequence encoding the inserted suppressor tRNA may be edited to comprise any suitable anticodon sequence capable of binding to a PTC. The DNA sequence encoding the inserted suppressor tRNA may further comprise any suitable edits to enable charging of the suppressor tRNA molecule with any suitable amino acid. Again, without wishing to be bound by any particular theory, it is believed that replacing the highly expressed tRNA gene with the suppressor tRNA gene will enhance expression of the desired suppressor tRNA molecule, and hence, result in increased read-through efficiency of targeted PTCs.

Additionally, aspects of the disclosure relate to inserting a new suppressor tRNA gene into a human genome. In some embodiments, the tRNA gene is inserted into a safe harbor locus (e.g., ROSA, CCR5, AAVS1, etc.) or general expression site (e.g., ALB) in a host genome (e.g., human. Without wishing to be bound by any particular theory, this approach requires insertion of a small gene (e.g., <1 kb) rather than a local edit of a subset of endogenous tRNA bases but may offer complementary advantages such as the lack of dependence on the presence, sequence, and dispensability of an endogenous tRNA gene in a specific target organism or patient. Further, because tRNAs are short (˜200 bp), and typical Pol III promoters for expressing short RNAs are also small (e.g., U6 promoter, 264 bp), it is possible that all of the elements required for suppressor tRNA expression could be inserted by prime editing methods, such as twin prime editing. Alternatively, in some embodiments, prime editing or twin prime editing is coupled with integrase or recombinase enzymes to perform the insertion as described in U.S. Patent Application Ser. No. 63/271,700, filed Oct. 25, 2021, and in PCT Patent Application, Serial Number PCT/US2022/078655, filed Oct. 25, 2022, both of which are herein incorporated by reference in their entirety. The use of CRISPR-associated transposases (CASTs) and other targeted gene insertion technologies to achieve insertion of a suppressor tRNA or a suppressor tRNA expression cassette into the genome is likewise also envisioned, in other embodiments.

In some embodiments, the methods and compositions comprise inserting the gene into the genome using twin prime editing (twinPE). Briefly, twinPE is an art recognized gene editing technique comprising a first prime editor complex and a second prime editor complex.

Each prime editing complex comprises a prime editor and a pegRNA. Each prime editor comprises a nucleic acid programmable DNA binding protein (napDNAbp) and a polypeptide having an RNA-dependent DNA polymerase activity. Each pegRNA comprises a spacer sequence, gRNA core, an extension arm comprising a DNA synthesis template and a primer binding site (PBS). The DNA synthesis template of the pegRNA of the first prime editor complex encodes a first single-stranded DNA sequence (e.g. desired insert in the 5′-3′ direction) and the DNA synthesis template of the pegRNA of the second prime editor complex encodes a second single-stranded DNA sequence (e.g., desired insert in the 3′-5′ direction). The first single-stranded DNA sequence and the second single-stranded DNA sequence each comprises a region of complementarity to the other, such that, the first single-stranded DNA sequence and the second single-stranded DNA sequence form a duplex comprising the desired insert, as compared to the DNA sequence at the target site to be edited, which integrates into the target site to be edited. In some embodiments, the spacer sequence and extension arm are any sequence listed in Table 2. In some embodiments, any one of the pegRNA sequences listed in Table 2 may further comprise a terminal 5′-G. Without wishing to be bound by any particular theory, it is believed that the 5′-G is necessary for use with polIII promoters, such as U6.

In some embodiments, the methods and compositions comprise inserting a suppressor tRNA gene into the genome using prime editing methods coupled with an integrase and/or a recombinase enzyme. Without wishing to be bound by any particular theory, recombinases, such as serine integrases (e.g., BxB1) are art recognized enzymes capable of performing site-specific recombination. Site-specific recombination is an art recognized process in which DNA strand exchange takes place between two DNA segments (e.g., two different double strand DNAs) possessing at least a certain degree of sequence homology. The enzymes recognize and bind to short specific DNA recognition sites (e.g., a first recognition site located on the first double stranded DNA, and a second recognition site located on a second double stranded DNA), at which they cleave the DNA backbone, exchange the two DNA helices involved, and rejoin the DNA strands. In some embodiments, the first and second recognition sites comprise identical sequences. In other embodiments, the first and second recognition sites comprise different sequences (e.g., attP and attB of phage integrase). Other suitable prime editing systems known by the skilled artisan may also be used, such as the multi-flap (e.g., dual-flap and/or quadruple-flap) prime editing systems disclosed in U.S. patent application Ser. No. 18/053,269 filed on Nov. 7, 2022 and published on Jul. 13, 2023 with Publication No.: US-2023-0220374-A1 and International Patent Application No.: PCT/US2021/031439 filed on May 7, 2021 and published on Nov. 11, 2021 with Publication No.: WO2021/226558, both of which are incorporated herein by reference in their entirety.

In some embodiments, the methods and compositions comprise a DNA plasmid (e.g., a circular plasmid) that encodes a suppressor tRNA comprising an anticodon sequence that is complementary to a PTC (e.g., 5′-UUA-3′, 5′-UCA-3′, 5′-CUA-3′). The DNA plasmid (e.g., circular plasmid) may further comprise a first recombinase recognition site (e.g., AttP). The recombination site may be any suitable recombination site known in the art. For example, in some cases, the first recombinase recognition site comprises an AttB sequence with a sequence identity of at least 85%, at least 90%, at least 95%, at least 98%, at least 99%, or at least 99.5% to SEQ ID NOs: 1-9 and 45711-45712. In other embodiments, the first recombinase recognition site comprises an AttP sequence with a sequence identity of at least 85%, at least 90%, at least 95%, at least 98%, at least 99%, or at least 99.5% to SEQ ID NOs: 10-17 and 45713-45715. Other first recombinase recognition sites are also possible, according to other embodiments.

Exemplary attB and attP sites for phage integrase C31 (φC31 attB) are shown below and are described in Groth et al., A phage integrase directs efficient site-specific integration in human cells” Proceedings of the National Academy of Science USA. May 23, 2000, vol. 97, no. 11, pgs. 5995-6000, and Anzalone et al., “Programmable deletion, replacement, integration, and inversion of large DNA sequences with twin prime editing” Nature Biotechnology. May 2022, 40(5): 731-740, both of which are incorporated herein by reference in their entirety. However, the skilled artisan will appreciate that the invention is not limited to phage integrases, and that any integrase with known attB and attP sites known by the skilled artisan may be used in the current disclosure.

(SEQ ID NO: 1)
CTCGA AGCCG CGGTG CGGGT GCCAG GGCGT GCCCT
TGGGC TCCCC GGGCG CGTAC TCCAC CTCAC CCATC
(SEQ ID NO: 2)
CCG CGGTG CGGGT GCCAG GGCGT GCCCTTGGGC TCCCC
GGGCG CGTAC TCCACC
(SEQ ID NO: 3)
CGGGT GCCAG GGCGT GCCCTTGGGC TCCCC GGGCG CGTAC
(SEQ ID NO: 4)
GGT GCCAG GGCGT GCCCTTGGGC TCCCC GGGCG CG
(SEQ ID NO: 5)
GT GCCAG GGCGT GCCCTTGGGC TCCCC GGGCG CG
(SEQ ID NO: 6)
GT GCCAG GGCGT GCCCTTGGGC TCCCC GGGCG C
(SEQ ID NO: 7)
T GCCAG GGCGT GCCCTTGGGC TCCCC GGGCG CG
(SEQ ID NO: 8)
T GCCAG GGCGT GCCCTTGGGC TCCCC GGGCG C
(SEQ ID NO: 9)
GGCGT GCCCTTGGGC TCCCC
(SEQ ID NO: 45711)
GGCTTGTCGACGACGGCGGACTCCGTCGTCAGGATCAT
(SEQ ID NO: 45712)
GGCTTGTCGACGACGGCGGTCTCCGTCGTCAGGATCAT
AttP sites
(SEQ ID NO: 10)
CGGGA GTAGT GCCCC AACTG GGGTA ACCTTTGAGT TCTCT
CAGTT GGGGG CGTAG GGTCG
(SEQ ID NO: 11)
GTAGT GCCCC AACTG GGGTA ACCTTTGAGT TCTCT CAGTT
GGGGG CGTAG
(SEQ ID NO: 12)
GCCCC AACTG GGGTA ACCTTTGAGT TCTCT CAGTT GGGGG
(SEQ ID NO: 13)
GCCCC AACTG GGGTA ACCTTTGAGT TCTCT CAGTT GGGG
(SEQ ID NO: 14)
CCCC AACTG GGGTA ACCTTTGAGT TCTCT CAGTT GGGGG
(SEQ ID NO: 15)
CCCC AACTG GGGTA ACCTTTGAGT TCTCT CAGTT GGGG
(SEQ ID NO: 16)
CCC AACTG GGGTA ACCTTTGAGT TCTCT CAGTT GGG
(SEQ ID NO: 17)
CC AACTG GGGTA ACCTTTGAGT TCTCT CAGTT GG
(SEQ ID NO: 45713)
GGTTTGTCTGGTCAACCACCGCGGACTCAGTGGTGTACGGTACAA
ACC
(SEQ ID NO: 45714)
GGTTTGTCTGGTCAACCACCGCGGTCTCAGTGGTGTACGGTACAAA
CC
(SEQ ID NO: 45715)
AGGTTTGTCTGGTCAACCACCGCGGTCTCAGTGGTGTACGGTACAAA
CCT

Additionally, the methods and compositions further comprise using prime editing to incorporate a second recombinase recognition site into a target site in the human genome (e.g., safe harbor locus site or general expression site). In some embodiments, the target site comprises a safe harbor site. Safe harbor sites are sites within a host genome that support stable and efficient transgene expression without detrimentally altering cellular function. Any safe harbor site may be used as the insertion point for any of the methods disclosed herein. In some embodiments, a pegRNA guides the prime editor to the target site and encodes the edit to be installed into the human genome. In some embodiments, the DNA synthesis template encodes a single stranded DNA sequence encoding a second recombinase recognition site (e.g., a AttP or AttB). In some embodiments, the second recombinase recognition site comprises an AttB sequence with a sequence identity of at least 85%, at least 90%, at least 95%, at least 98%, at least 99%, or at least 99.5% to SEQ ID NOs: 1-9 and 45711-45712. In some embodiments, the second recombinase recognition site comprises an AttP sequence with a sequence identity of at least 85%, at least 90%, at least 95%, at least 98%, at least 99%, or at least 99.5% to SEQ ID NOs: 10-17 and 45713-45715.

Those of skill in the art will understand, based on the above discussion, that the integrase recombines the DNA plasmid (e.g., circular plasmid) comprising the first recombination recognition site with the second recombination site, previously inserted into the human genome at the target site (e.g., safe harbor locus) via prime editing, to permanently insert the desired suppressor tRNA gene into the human genome at the target site (e.g., ROSA26, CCR5, and AAVS1). In some embodiments, installation of the suppressor tRNA gene at the target site (e.g., safe harbor locus site or general expression site) results in the indefinite expression of the suppressor tRNAs gene.

Other aspects of the present disclosure relate to methods and compositions comprising prime editors. The methods and compositions disclosed herein may comprise any suitable prime editor known to those of skill in the art, such as those disclosed in Chen and Li, “Prime editing for precise and highly versatile genome manipulation” Nature Review Genetics, 2022, doi.org/10.1038/s41576-022-00541-1, which is incorporated herein by reference in its entirety Exemplary embodiments include, but are not limited to, PE1, PE2, PE2*, PEmax, CMP-PE1-V1, hyPE2, IN-PE, ePPE, PE-P3, PE2^{ΔRnh}, sPE, nCas9 and MCP-RT, PE4/PE5, PE2-VQR, PE2-VRQR, PE2-VRER, PE2-NG, PE2-SpG, PE2-SpRY, SaPE2, SaPE2*, Sa^{KKH}PE2, Sa^{KKH}PE2*, SauriCas9-PE, CjCas9-PE, and FnCas9-PE.

In some embodiments, the prime editor may be a PE2, PE3, PE4, PE5, PE2max, PE3max, PE4max, PE5max, twinPE, or Prime-del, such as those disclosed in U.S. Patent Application Ser. No. 63/022,397, filed May 8, 2020, U.S. Patent Application Ser. No. 63/116,785, filed Nov. 20, 2020, and PCT Application, Serial Number PCT/US2021/031439, filed May 7, 2021, each of which is incorporated herein by reference in its entirety.

In some embodiments, the prime editors disclosed herein comprise either an SaCas9 or SpCas9, or a derivative thereof, fused to PEmax or PE6 (e.g., PE6a, PE6b, PE6c, PE6d, PE6e, PE6f, PE6g).

Additionally, some aspects of the present disclosure relate to methods and compositions comprising one or more pegRNAs. Any suitable pegRNA may be used by the skilled artisan, such as those disclosed by Chen and Liu (Nature Review Genetics, 2022). In some embodiments, the pegRNAs comprise a spacer sequence. As is common knowledge in the art, the spacer sequence is configured to bind a target DNA sequence. Any suitable spacer sequence known to the skilled artisan may be used herein, such as those shown in Table 2. In some embodiments, the pegRNA further comprises a 3′-tevopreQi motif stabilizing motif that improves editing efficiency as described in U.S. Patent Application Ser. No. 63/477,155, filed Dec. 23, 2022, which is herein incorporated by reference in its entirety.

In some embodiments, the pegRNAs comprises an extension arm. As is common knowledge in the art, the extension arm comprises a DNA synthesis template that encodes the one or more edits to be installed at the DNA target site and a primer binding site (PBS). Any suitable extension arm known to the skilled artisan may be used herein, such as those shown in Table 2. In some embodiments, the pegRNA directs the prime editor to install an edit at a target site located between positions +11 and +17, relative to a first editable base located 3′ of a pegRNA-directed nick.

In some embodiments, the DNA synthesis template encodes an ochre PTC sequence, an opal PTC sequence, or an amber PTC sequence (e.g., in the 3′ to 5′ direction) to be installed at the target site of the DNA sequence encoding the anticodon sequence domain of the tRNA. Briefly, the DNA synthesis template encodes the PTC in the 3′ to 5′ direction and installs the PTC anticodon sequence (via a polymerase) into the DNA sense strand encoding the endogenous tRNA anticodon sequence. The edit is subsequently incorporated into the endogenous tRNA following a series of process including hybridization, flap cleavage, ligation, and mismatch repair. This results in the installation of PTC in the antisense strand of the DNA sequence encoding the tRNA, which following transcription produces a suppressor tRNA comprising an anticodon to the PTC.

In some embodiments, the DNA synthesis template encodes a nonsense suppressor codon to be installed at a target site of a DNA sequence (e.g., sense strand or coding strand) encoding the anticodon sequence of the tRNA. In some embodiments, the nonsense suppressor codon is selected from the group consisting of 5′-UAA-3′, 5′-UGA-3′, and 5′-UAG-3′.

Alternatively, In some embodiments, the DNA synthesis template encodes a nonsense suppressor anticodon sequence to be inserted into an anticodon sequence of the suppressor tRNA molecule, relative to an endogenous, dispensable tRNA molecule. In this set of embodiments, the DNA synthesis template encodes the anticodon in the 5′ to 3′ direction and installs the PTC into the antisense strand encoding the endogenous tRNA anticodon sequence. The edit is subsequently incorporated into the endogenous tRNA following a series of processes including hybridization, flap cleavage, ligation, and mismatch repair. This results in the installation of anticodon in the antisense strand of the DNA sequence encoding the tRNA, which following transcription produces a suppressor tRNA comprising an anticodon to the PTC. In some embodiments, the nonsense suppressor anticodon is selected from the group consisting of 5′-UUA-3′, 5′-UCA-3′, and 5′-CUA-3′.

Regardless of the mechanism, the DNA synthesis template contains the genetic information necessary (e.g. either CTA/TAG, TCA/TGA, TTA/TAA depending on the strand that the pegRNA binds to), in combination with the prime editor, to ensure that the final anticodon of the tRNA will be the reverse complement (e.g., CTA, TCA, or TTA) of one of the three premature termination codons (e.g., TAG, TGA, or TA A).

In some embodiments, the DNA synthesis template encodes a C70U mutation to be installed at the target site of the DNA sequence encoding the acceptor stem domain of the endogenous, dispensable tRNA. In some embodiments, the tRNA is an endogenous indispensable tRNA. Without wishing to be bound by theory, it is believed that the C70U mutation installs a G3:U70 base pair in the acceptor stem domain, which preferentially is charged by the alanine-aminoacyl-tRNA synthetase. In some embodiments, the DNA synthesis template encodes a single base nucleotide insertion to be installed at the target site of the DNA sequence encoding the variable arm domain of the tRNA. In some embodiments, the DNA synthesis template further encodes a PAM-disrupting mutation, an MMR-evading mutation, or any combination thereof, relative to the endogenous tRNA gene.

In some embodiments, the pegRNA further comprises a protospacer sequence. It is common knowledge in the art that the protospacer sequence binds to a target DNA sequence of interest. Any suitable protospacer known to the skilled artisan may be used to bind to the target tRNA, such as any protospacer sequence listed in Table 2. In some embodiments, the pegRNA is chosen to enable editing of a single specified endogenous tRNA gene without editing other tRNA genes, especially by avoiding the targeting of regions that are highly homologous among tRNA genes. Without wishing to be bound by any particular theory, it is believed that because of prime editing's exquisite target specificity, pegRNAs may be carefully designed to distinguish between on-target and off-target tRNA genes, even ones that differ at most by a few base pairs.

Aspects of the disclosure relate to a complex comprising a prime editor and any pegRNA described herein. In some embodiments, pegRNA comprises a spacer sequence. Any suitable spacer sequence known in the art may be used, such as any sequence listed in Table 2. In some embodiments, the pegRNA comprises an extension arm. Any suitable extension arm known to the skilled artisan may be used, such as any sequence listed in Table 2. In some embodiments, the pegRNA comprises a spacer sequence and an extension arm, such as those listed in Table 2. In some embodiments, the spacer sequence and/or extension arm are at least 85%, at least 90%, at least 95%, at least 98%, at least 99%, or at least 99.5% identical to any sequence listed in Table 2.

In some embodiments, the complex comprises a prime editor and multiple pegRNA's. In some embodiments, the complex comprises a pair of pegRNA's (e.g., two pegRNA's). In some embodiments, the complex comprises two pairs of pegRNA's (e.g., four pegRNA's). Non-limiting embodiments of pairs of pegRNAs designed, for example, to replace an endogenous tRNA sequence with a suppressor tRNA sequence via prime editing, may be found in Table 5.

Other aspects of the disclosure relate to polynucleotides, vectors, cells, pharmaceutical compositions, and kits. For example, in some aspects, the disclosure relates to a polynucleotide comprising a first nucleic acid sequence encoding a prime editor and a second nucleic acid sequence encoding a pegRNA. In some embodiments, the pegRNA comprises any spacer sequence and/or an extension arm listed in Table 2. In some embodiments, the spacer sequence and/or extension arm are at least 85%, at least 90%, at least 95%, at least 98%, at least 99%, or at least 99.5% identical to any sequence listed in Table 2.

In some aspects, the disclosure relates to cells comprising any one of the polynucleotides, complexes, pegRNAs, and/or vectors disclosed herein

In some aspects, the disclosure relates to pharmaceutical compositions comprising any one of the compositions, pegRNAs, complexes, polynucleotides, vectors, and cells disclosed herein, or any combination thereof, and a pharmaceutically acceptable excipient.

In some aspects, the disclosure relates to kits comprising any one of the compositions (e.g., pharmaceutical compositions), pegRNAs, complexes, polynucleotides, vectors, and cells disclosed herein, or any combination thereof, and instructions for editing one or more DNA sequences encoding one or more domains of a tRNA by prime editing. In some embodiments, the tRNA DNA sequence is any sequence listed in Table 1. In some embodiments, the tRNA DNA sequence is at least 85%, at least 90%, at least 95%, at least 98%, at least 99%, or at least 99.5% identical to any sequence listed in Table 1.

Aspects of the present disclosure also relate to methods for selecting suppressor tRNAs with high read-through efficiency. In some embodiments, the methods comprise creating a reporter cell line comprising a reporter construct comprising a constitutively expressed fusion protein comprising a first biomarker protein (e.g., first fluorescent protein), a premature termination codon (PTC) sequence, a ribosomal skipping element, and a second biomarker protein (e.g., second fluorescent protein) different than the first biomarker protein (e.g., first fluorescent protein). The biomarker protein can be any suitable biomarker protein known to those of skill in the art, such as, but not limited to, a fluorescent protein, a phosphorescence protein, or chemiluminescence protein. In some cases, the biomarker protein is mCherry and/or green fluorescent protein. In some embodiments, the methods comprise creating a gene library encoding the sequences of all human tRNA sequences, wherein all the tRNA sequences comprise the same three-base pair anticodon that is complementary to the PTC in the reporter construct. In some embodiments, the methods comprise introducing the library into the reporter cell line, sorting cells that express the second biomarker protein (e.g., second fluorescent protein), and determining which tRNA sequences are enriched in the sorted population.

Other methods relate to methods and compositions for selecting pegRNAs to edit endogenous tRNA genes into suppressor tRNA genes. In some cases, the methods comprise creating a reporter cell line comprising a reporter construct comprising a constitutively expressed fusion protein comprising a first biomarker protein (e.g., fluorescent), a premature termination codon (PTC) sequence, a ribosomal skipping element, and a second biomarker protein (e.g., fluorescent) different than the first biomarker protein (e.g., fluorescent). In some embodiments, the methods further comprise creating a gene library (>22,000) encoding pegRNAs that target every tRNA sequence in the genome. The pegRNAs further comprise an extension arm encoding the genetic information needed to convert the natural tRNA anticodon to a PTC anticodon (e.g., 5′-CTA-3′) to recognize the PTC (e.g., an amber stop codon, 5′-TAG-3′). In some embodiments, the methods comprise introducing the gene library and a prime editor into the cell line, sorting cells that express the second biomarker protein (e.g., fluorescent biomarker), and determining which pegRNA sequences are enriched in the sorted population.

Additional aspects relate to methods for treating a disease caused by premature termination codons, the method comprising installing a suppressor tRNA gene into a target site in a human genome using prime editing, the method comprising administering to a subject (i) a prime editor and (ii) a pegRNA, wherein the suppressor tRNA gene encodes a suppressor tRNA molecule comprising an anticodon sequence comprising ochre stop codon, an opal stop codon, or an amber stop codon.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

The strategies described above are not intended to be limiting in any way. As such, the any one of the disclosed strategies may be used independently or in combination with one or more of the other strategies. For example, in some embodiments, the strategy may comprise using prime editing to edit one or more domains of the tRNA molecule (e.g., anticodon domain and the acceptor stem domain). In some embodiments, the strategy may comprise editing a DNA sequence encoding endogenous tRNA to produce a suppressor tRNA comprising an anticodon that is complimentary to a PTC and charged with a non-cognate amino acid. In some embodiments, an endogenous tRNA isodecoder gene is replaced with a suppressor tRNA gene charged with non-cognate amino acid using prime editing. Other embodiments are also envisioned and are discussed in detail elsewhere herein.

### BRIEF DESCRIPTION OF DRAWINGS

The following drawings form part of the present specification and are included to further demonstrate certain aspects of the present disclosure, which may be better understood by reference to one or more of these drawings in combination with the detailed description of specific embodiments presented herein.

FIG. 1 shows a schematic illustrating the creation of suppressor tRNAs from endogenous tRNA genes using prime editing.

FIG. 2 shows the editing efficiency of 11 endogenous tRNAs following prime editing conversion to suppressor tRNAs in HEK293T cells. Edits include: Arg-CCT-5-1 (CCT>TCA), Arg-CCT-5-1 (CCT>CTA), Arg-CCG-2-1 (CCG>CTA), Arg-CCG-2-1 (CCG>TCA), Arg-TCT-4-1 (TCT>TCA); Arg-TCT-1-1 (TCT>TCA), Lys-CTT-3-1 (CTT>TCA). Lys-CTT-15-1 (CTT>TCA). Lys-CTT-15-1 (CTT>CTA), Leu-CAA-6-1 (CAA>TCA), Leu-CAA-6-1 (CAA>CTA), Leu-TAA-2-1 (TAA>TCA), Leu-TAA-2-1 (TAA>CTA), Leu-TAG-3-1 (TAG>TCA), Leu-TAG-3-1 (TAG>CTA), Gln-CTG-5-1 (CTG>TCA), Gln-CTG-5-1 (CTG>CTA), Ala-AGC-5-1 (AGC>CTA), and Ala-AGC-5-1 (AGC>TCA).

FIG. 3A shows an illustration of a generalized reporter assay used to determine the readthrough efficiency following prime editing conversion of endogenous tRNAs into suppressor tRNAs.

FIG. 3B shows a plot of the percent of sequencing reads with the specified edit or indels for Arg-CCG-2-1 (CCG>CTA) and Leu-TAA-2-1 (TAA>TCA) using prime editing in HEK293T cells.

FIG. 3C shows a plot of the percentage of fluorescent cells obtained using the reporter assay shown in FIGS. 3A and 3B. The eGFP reporter plasmid was edited to contain a single premature termination codon (PTC) located at R109X or L42X. Fluorescent cells are the result of PTC readthrough.

FIG. 4 shows a representative schematic of an exemplary endogenous, dispensable tRNA. Relevant domains include the D-arm domain (e.g., D-loop), acceptor stem domain, T-arm domain (e.g., TΨC loop), variable arm domain (e.g., variable loop), and the anticodon arm domain encoding the anticodon sequence (e.g., anticodon loop) (SEQ ID NO: 45704).

FIGS. 5A-5C show that editing tRNA-Leu-TAA-2-1 (FIG. 5A) (from top to bottom, SEQ ID NOs: 4570545706) leads to detectable read through at mRNA (FIG. 5B) but not protein levels (FIG. 5C).

FIGS. 6A-6D show that each Leu-TAA tRNA gene (FIG. 6A) (from top to bottom, SEQ ID NOs: 45707-45710) can be specifically targeted using prime editing (FIGS. 6B-6D).

FIG. 6B shows editing efficiency of uniquely targeted Leu-TAA tRNA gene family members.

FIG. 6C shows readthrough efficiency measured by percentage of GFP positive single-integrant HEK293T reporter cells following PERT treatment. FIG. 6D shows exemplary quantification of GFP positive cells using flow cytometry.

FIGS. 7A-7B illustrate that converting endogenous Leu-TAA tRNA genes into suppressors using PERT rescues protein expression at two different disease loci (Niemann-Pick disease type C) in HEK293 cells.

FIGS. 8A-8B show that endogenous tRNAs are expressed at different levels (FIG. 8A) and with different number of isodecoders (FIG. 8B)

FIGS. 9A and 9B illustrate that overwriting endogenous tRNA genes (SER-GCT-3-1 or Cys-GCA-3-1) with suppressor tRNA sequences (Leu-TAA-3-1) elicits readthrough of PTCs. Editing efficiencies of tRNA genes are shown in FIG. 9A. Readthrough efficiency measured by median fluorescent signal from single-integrant HEK293T reporter cell populations subject to twinPE-mediated tRNA gene replacement are shown in FIG. 9B.

FIGS. 10A-10B illustrate that overwriting endogenous tRNA genes (Ser-GCT-3-1) with suppressor tRNA sequences (Leu-TAA-4-1) elicits readthrough of PTCs. Editing efficiencies of tRNA genes are shown in FIG. 10A. Readthrough efficiency measured by median fluorescent signal from single-integrant HEK293T reporter cell populations subject to twinPE-mediated tRNA gene replacement are shown in FIG. 10B.

FIG. 11 outlines the reporter construct used to monitor PTC-containing protein translation readthrough. The lentiviral reporter construct contains an mCherry fluorescent protein followed by a premature termination codon (PTC), a ribosomal skipping element (2a), and a GFP fluorescent protein.

FIGS. 12A-12D show the percentage of GFP+ cells when the codon containing a PTC in the lentiviral reporter construct is switched to any of the 20 amino acids at PTC location 1 (FIG. 12B), PTC location 2 (FIG. 12C), and PTC location 3 (FIG. 12D).

FIG. 13 shows the screening strategy used to compare the ability of different suppressor tRNA variants to enable PTC readthrough. The lentiviral tRNA screening construct containing a library of suppressor tRNA variants and one of three promoters: a human U6 promoter, a minimal U6 promoter, or no exogenous promoter beyond the endogenous promoter elements embedded within the tRNA.

FIGS. 14A-14C show results of quality control experiments performed of candidate suppressor tRNA screening plasmid pools. FIG. 14A shows a plot of the percentage of individually miniprepped colonies as a function of promoter backbone that contain the correct versus incorrect sequence. FIG. 14B shows a plot of the number of alignments as a function of the promoter backbone. FIG. 14C shows a plot of the perfect match as a function of the promoter backbone.

FIG. 15 shows that when a premature termination codon is installed before GFP in an mCherry-2a-GFP mRNA construct, a 10-fold lower protein expression of mCherry is observed. Without wishing to be bound by any particular theory, it is hypothesized that this is due to nonsense-mediated mRNA decay. Typically, nonsense-mediated decay is initiated by factors that are recruited to splice sites that are not present in the lentivirus construct. Therefore, it is suspected that this effect is being induced by the lack of a polyA tail in the lentiviral construct, which results in a long 3′-UTR that can also be a substrate for the proteins required to initiate nonsense-mediated decay.

FIG. 16 shows exemplary flow cytometry results illustrating readthrough with hU6 and min-hU6 promoter sup-tRNA pools and TAG reporter systems.

FIGS. 17A-17B show results after sorting the top 5% (FIG. 17A) and 0.5% (FIG. 17B) GFP+ cells that exhibited readthrough with the reporter construct following transduction with the lentiviral library of suppressor tRNAs. The theoretical maximum enrichment value is 200-fold. Results from suppressor tRNAs preceded by a human U6 promoter (top), a minimal U6 promoter (middle), and no exogenous promoter (bottom) are shown.

FIGS. 18A-18B show that 40-bp leader sequences that precede endogenous tRNAs are important for regulating suppressor tRNA function. (FIG. 18A) Pool of lentiviral constructs containing a suppressor tRNA (Leu-TAA-4-1 with anticodon switched to CTA) or control tRNA (Leu-TAA-4-1 with native anticodon) as well as the 40-bp leader sequences that precede every endogenous tRNA in the genome. (FIG. 18B) Enrichment of leader sequences that precede the control construct (left) or the suppressor tRNA construct (right) in the GFP+ population. Bulk GFP+ cells represent all cells exhibiting readthrough (˜44% of cells, with a theoretical maximum enrichment value of 2.27-fold), whereas the [0-25%1, 125-50%], [50-75%], and [75-100%] populations represent quadrants of GFP+ cells (25% of the ˜44% of GFP+ cells, with a theoretical maximum enrichment value of 9-fold).

FIG. 19 shows that termination sequences are required for activity of hU6-Leu-TAA-3 and hU6-Leu-TAA-4 suppressor tRNAs.

FIGS. 20A-20D show the results from a pegRNA screen to identify optimal suppressor tRNA sequences installed using PERT. Schematic of pegRNA screen. 22,177 PE2 epegRNAs targeting tRNAs and converting their anticodons to CTA as well as 1,616 control epegRNAs were packaged into lentivirus and transduced into 293T reporter cells. Cells were transfected with PEmax prime editor to initiate prime editing and cells exhibiting GFP+ readthrough were sorted and processed for next generation sequencing (FIG. 20A). Fold enrichment of pegRNAs targeting each of the amino acid families. Amino acid and anticodon sequence are indicated on the x-axis (FIG. 20B). Example of the performance of pegRNAs targeting one tRNA in the screen (Leu-AAG-2-1) with various RTT homology and PBS lengths (FIG. 20C). Fold enrichment of pegRNAs targeting each of the amino acid families introducing a TGA suppressor anticodon with amino acid and anticodon sequences as indicated on the x-axis (FIG. 20D).

FIG. 21 shows the results from saturation mutagenesis of the Leu-TAA-4-1 suppressor tRNA.

FIG. 22 shows the results of a study in which tRNA expression with native leader sequences was determined. The results indicate that the best leader sequences seem to precede highly expressed tRNAs in 293T cells.

FIGS. 23A-23C show that tRNA sequences can be installed into safe harbor loci with high frequency using twinPE. FIG. 23A shows results for experiments employing a 20 bp overlap, while FIG. 23B shows results for experiments using a 30 bp overlap. FIG. 23C shows data demonstrating successful twinPE installation of a mature suppressor tRNA sequence.

FIGS. 24A-24D show variants identified in a saturation mutagenesis screen enhance readthrough in Leu-TAA-1-1 and Leu-TAA-3-1. Editing efficiency of Leu-TAA-1-1 to introduce an anticodon edit, or an anticodon edit and the variants indicated on the x-axis (FIG. 24A). Readthrough efficiency measured by percentage of GFP positive single-integrant HEK293T reporter cells following PERT treatment introducing additional sequence variants in Leu-TAA-1-1 (FIG. 24B). Editing efficiency of Leu-TAA-3-1 to introduce an anticodon edit, or an anticodon edit and the variants indicated on the x-axis (FIG. 24C). Readthrough efficiency measured by percentage of GFP positive single-integrant HEK293T reporter cells following PERT treatment introducing additional sequence variants in Leu-TAA-3-1 (FIG. 24D)

FIGS. 25A-25C show that the introduction of variant sequences identified in saturation mutagenesis screen enhances readthrough. FIG. 25A shows readthrough efficiency by western blot following delivery of epegRNA and ngRNA pairs capable of introducing a change of hp13 from G•C to T•A (a top hit from the validation in the reporter cell line described in FIG. 24) to HEK293T Niemann-Pick disease type C cell models. The introduction of the hairpin change alongside the anticodon edit led to a marked increase in full-length NPC1 protein production, reaching approximately 1% of wildtype control expression. FIG. 25B shows prime editing efficiency achieved for the indicated suppressor mutations in a mouse Neuro-2a cell model of Hurler syndrome. FIG. 25C shows the editing efficiency and corresponding readthrough in a TGA reporter cell line.

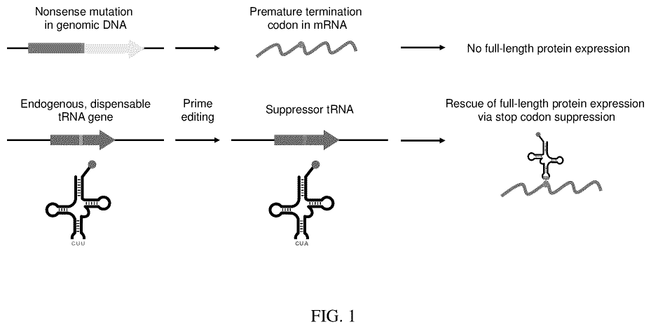

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

### Cas9

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain” as used herein, is a protein fragment comprising an active or inactive cleavage domain of Cas9 and/or the gRNA binding domain of Cas9. A “Cas9 protein” is a full length Cas9 protein. A Cas9 nuclease is also referred to sometimes as a casnl nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc), and a Cas9 domain. The tracrRNA serves as a guide for ribonuclease 3-aided processing of pre-crRNA. Subsequently, Cas9/crRNA/tracrRNA endonucleolytically cleaves a linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which are hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type H CRISPR-Cas immunity systems” (2013) RNA Biology 10:5, 726-737; the entire contents of which are incorporated herein by reference. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science. 337:816-821(2012); Qi et al., “Repurposing CRISPR as an RNA-Guided Platform for Sequence-Specific Control of Gene Expression” (2013) Cell. 28:152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to wild type Cas9 (e.g., SpCas9 of SEQ ID NO: 18). In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9 (e.g., SpCas9 of SEQ ID NO: 18). In some embodiments, the Cas9 variant comprises a fragment of SEQ ID NO: 18 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9 (e.g., SpCas9 of SEQ ID NO: 18). In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SpCas9 of SEQ ID NO: 18).

The skilled artisan will appreciate that any wild type Cas9 or derivative thereof known to the skilled artisan may be used as disclosed herein, such as, for example:

SpCas9, Streptococcus pyogenes M1,
SwissProt Accession No. Q99ZW2, Wild type
(SEQ ID NO: 18)
MDKKYSIGLDIGTNSVGWAVITDEYKVPSKKFKVLGNTDRHSIKK
NLIGALLFDSGETAEATRLKRTARRRYTRRKNRICYLQEIFSNEM
AKVDDSFFHRLEESFLVEEDKKHERHPIFGNIVDEVAYHEKYPTI
YHLRKKLVDSTDKADLRLIYLALAHMIKFRGHFLIEGDLNPDNSD
VDKLFIQLVQTYNQLFEENPINASGVDAKAILSARLSKSRRLENL
IAQLPGEKKNGLFGNLIALSLGLTPNFKSNFDLAEDAKLQLSKDT
YDDDLDNLLAQIGDQYADLFLAAKNLSDAILLSDILRVNTEITKA
PLSASMIKRYDEHHQDLTLLKALVRQQLPEKYKEIFFDQSKNGYA
GYIDGGASQEEFYKFIKPILEKMDGTEELLVKLNREDLLRKQRTF
DNGSIPHQIHLGELHAILRRQEDFYPFLKDNREKIEKILTFRIPY
YVGPLARGNSRFAWMTRKSEETITPWNFEEVVDKGASAQSFIERM
TNEDKNLPNEKVLPKHSLLYEYFTVYNELTKVKYVTEGMRKPAFL
SGEQKKAIVDLLFKTNRKVTVKQLKEDYFKKIECFDSVEISGVED
RFNASLGTYHDLLKIIKDKDFLDNEENEDILEDIVLTLTLFEDRE
MIBERLKTYAHLFDDKVMKQLKRRRYTGWGRLSRKLINGIRDKQS
GKTILDFLKSDGFANRNFMQLIHDDSLTFKEDIQKAQVSGQGDSL
HEHIANLAGSPAIKKGILQTVKVVDELVKVMGRHKPENIVIEMAR
ENQTTQKGQKNSRERMKRIEEGIKELGSQILKEHPVENTQLQNEK
LYLYYLQNGRDMYVDQELDINRLSDYDVDHIVPQSFLKDDSIDNK
VLTRSDKNRGKSDNVPSEEVVKKMKNYWRQLLNAKLITQRKFDNL
TKAERGGLSELDKAGFIKRQLVETRQITKHVAQILDSRMNTKYDE
NDKLIREVKVITLKSKLVSDFRKDFQFYKVREINNYHHAHDAYLN
AVVGTALIKKYPKLESEFVYGDYKVYDVRKMIAKSEQEIGKATAK
YFFYSNIMNFFKTEITLANGEIRKRPLIETNGETGEIVWDKGRDF
ATVRKVLSMPQVNIVKKTEVQTGGFSKESILPKRNSDKLIARKKD
WDPKKYGGFDSPTVAYSVLVVAKVEKGKSKKLKSVKELLGITIME
RSSFEKNPIDFLEAKGYKEVKKDLIIKLPKYSLFELENGRKRMLA
SAGELQKGNELALPSKYVNFLYLASHYEKLKGSPEDNEQKQLFVE
QHKHYLDEIIEQISEFSKRVILADANLDKVLSAYNKHRDKPIREQ
AENIIHLFTLTNLGAPAAFKYFDTTIDRKRYTSTKEVLDATLIHQ
SITGLYETRIDLSQLGGD

### Cognate Amino Acid

The term “cognate amino acid” refers to an amino acid that is conjugated to a tRNA molecule comprising an anticodon sequence encoding for said amino acid.

### CRISPR

CRISPR is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (mc) and a Cas9 protein. The tracrRNA serves as a guide for ribonuclease 3-aided processing of pre-crRNA. Subsequently, Cas9/crRNA/tracrRNA endonucleolytically cleaves a linear or circular dsDNA target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species—the guide RNA. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase Ill.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

In certain types of CRISPR systems (e.g., type II CRISPR systems), correct processing of pre-crRNA requires a trans-encoded small RNA (tracrRNA), endogenous ribonuclease 3 (mc), and a Cas9 protein. The tracrRNA serves as a guide for ribonuclease 3-aided processing of pre-crRNA. Subsequently, Cas9/crRNA/tracrRNA endonucleolytically cleaves a linear or circular nucleic acid target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered to incorporate embodiments of both the crRNA and tracrRNA into a single RNA species—the guide RNA.

In general, a “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. The tracrRNA of the system is complementary (fully or partially) to the tracr mate sequence present on the guide RNA.

### DNA Synthesis Template

As used herein, the term “DNA synthesis template” refers to the region or portion of the extension arm of a pegRNA that is utilized as a template strand by a polymerase of a prime editor to encode a 3′ single-strand DNA flap that contains the desired edit and which then, through the mechanism of prime editing, replaces the corresponding endogenous strand of DNA at the target site. The extension arm, including the DNA synthesis template, may be comprised of DNA or RNA. In the case of RNA, the polymerase of the prime editor can be an RNA-dependent DNA polymerase (e.g., a reverse transcriptase). In the case of DNA, the polymerase of the prime editor can be a DNA-dependent DNA polymerase. In various embodiments the DNA synthesis template may comprise the “edit template” and the “homology arm”, and all or a portion of the optional 5′ end modifier region, e2. That is, depending on the nature of the e2 region (e.g., whether it includes a hairpin, toe loop, or stem/loop secondary structure), the polymerase may encode none, some, or all of the e2 region as well. Said another way, in the case of a 3′ extension arm, the DNA synthesis template can include the portion of the extension arm that spans from the 5′ end of the primer binding site (PBS) to 3′ end of the gRNA core that may operate as a template for the synthesis of a single-strand of DNA by a polymerase (e.g., a reverse transcriptase). In the case of a 5′ extension arm, the DNA synthesis template can include the portion of the extension arm that spans from the 5′ end of the pegRNA molecule to the 3′ end of the edit template. Preferably, the DNA synthesis template excludes the primer binding site (PBS) of pegRNAs either having a 3′ extension arm or a 5′ extension arm. Certain embodiments described here refer to an “an RT template,” which is inclusive of the edit template and the homology arm, i.e., the sequence of the pegRNA extension arm which is actually used as a template during DNA synthesis. The term “RT template” is equivalent to the term “DNA synthesis template.”

### Dual Prime Editing

As used herein, the terms “dual prime editing”, “twin prime editing (or twinPE)”, and “dual-flap prime editing” are considered equivalent. In the dual-flap prime editing system, two pegRNAs are used to target opposite strands of a genomic site and direct the synthesis of two complementary 3′ flaps containing edited DNA sequence. Unlike classical prime editing, there is no requirement for the pair of edited DNA strands (3′ flaps) to directly compete with 5′ flaps in endogenous genomic DNA, as the complementary edited strand is available for hybridization instead. Since both strands of the duplex are synthesized as edited DNA, the dual-flap prime editing system obviates the need for the replacement of the non-edited complementary DNA strand required by classical prime editing. Instead, cellular DNA repair machinery need only excise the paired 5′ flaps (original genomic DNA) and ligate the paired 3′ flaps (edited DNA) into the locus. Therefore, there is no need to include sequences homologous to genomic DNA in the newly synthesized DNA strands, allowing selective hybridization of the new strands and facilitating edits that contain minimal genomic homology. Nuclease-active versions of prime editors that cut both strands of DNA could also be used to accelerate the removal of the original DNA sequence.

### Edit Template

The term “edit template” refers to a portion of the extension arm that encodes the desired edit in the single strand 3′ DNA flap that is synthesized by the polymerase, e.g., a DNA-dependent DNA polymerase, RNA-dependent DNA polymerase (e.g., a reverse transcriptase). Certain embodiments described here refer to “an RT template,” which refers to both the edit template and the homology arm together, i.e., the sequence of the pegRNA extension arm which is actually used as a template during DNA synthesis. The term “RT edit template” is also equivalent to the term “DNA synthesis template,” but wherein the RT edit template reflects the use of a prime editor having a polymerase that is a reverse transcriptase, and wherein the DNA synthesis template reflects more broadly the use of a prime editor having any polymerase.

### Extension Arm

The term “extension arm” refers to a nucleotide sequence component of a pegRNA which provides several functions, including a primer binding site and an edit template for reverse transcriptase. In some embodiments, the extension arm is located at the 3′ end of the guide RNA. In other embodiments, the extension arm is located at the 5′ end of the guide RNA. In some embodiments, the extension arm also includes a homology arm. In various embodiments, the extension arm comprises the following components in a 5′ to 3′ direction: the homology arm, the edit template, and the primer binding site. Since polymerization activity of the reverse transcriptase is in the 5′ to 3′ direction, the preferred arrangement of the homology arm, edit template, and primer binding site is in the 5′ to 3′ direction such that the reverse transcriptase, once primed by an annealed primer sequence, polymerizes a single strand of DNA using the edit template as a complementary template strand. Further details, such as the length of the extension arm, are described elsewhere herein.

The extension arm may also be described as comprising generally two regions: a primer binding site (PBS) and a DNA synthesis template, for instance. The primer binding site binds to the primer sequence that is formed from the endogenous DNA strand of the target site when it becomes nicked by the prime editor complex, thereby exposing a 3′ end on the endogenous nicked strand. As explained herein, the binding of the primer sequence to the primer binding site on the extension arm of the pegRNA creates a duplex region with an exposed 3′ end (i.e., the 3′ of the primer sequence), which then provides a substrate for a polymerase to begin polymerizing a single strand of DNA from the exposed 3′ end along the length of the DNA synthesis template. The sequence of the single strand DNA product is the complement of the DNA synthesis template. Polymerization continues towards the 5′ of the DNA synthesis template (or extension arm) until polymerization terminates. Thus, the DNA synthesis template represents the portion of the extension arm that is encoded into a single strand DNA product (i.e., the 3′ single strand DNA flap containing the desired genetic edit information) by the polymerase of the prime editor complex and which ultimately replaces the corresponding endogenous DNA strand of the target site that sits immediately downstream of the PE-induced nick site. Without being bound by theory, polymerization of the DNA synthesis template continues towards the 5′ end of the extension arm until a termination event. Polymerization may terminate in a variety of ways, including, but not limited to (a) reaching a 5′ terminus of the pegRNA (e.g., in the case of the 5′ extension arm wherein the DNA polymerase simply runs out of template), (b) reaching an impassable RNA secondary structure (e.g., hairpin or stem/loop), or (c) reaching a replication termination signal, e.g., a specific nucleotide sequence that blocks or inhibits the polymerase, or a nucleic acid topological signal, such as, supercoiled DNA or RNA.

### Fusion Protein

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. Another example includes a Cas9 or equivalent thereof to a reverse transcriptase. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

### General Expression Site

The term “general expression site” refers to a site in the human genome to which a gene of interest may be inserted, wherein the site is constitutively expressed (e.g., albumin gene, ALB).

Guide RNA (“gRNA”)

As used herein, the term “guide RNA” is a particular type of guide nucleic acid which is mostly commonly associated with a Cas protein of a CRISPR-Cas9 and which associates with Cas9, directing the Cas9 protein to a specific sequence in a DNA molecule that includes complementarity to the protospacer sequence of the guide RNA. However, this term also embraces the equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference. Exemplary sequences are and structures of guide RNAs are provided herein. In addition, methods for designing appropriate guide RNA sequences are provided herein. As used herein, the “guide RNA” may also be referred to as a “traditional guide RNA” to contrast it with the modified forms of guide RNA termed “prime editing guide RNAs” (or “pegRNAs”).

Guide RNAs or pegRNAs may comprise various structural elements that include, but are not limited to:

Spacer sequence—the sequence in the guide RNA or pegRNA (having about 20 nts in length) which binds to the protospacer in the target DNA.

gRNA core (or gRNA scatfold or backbone sequence)—refers to the sequence within the gRNA that is responsible for Cas9 binding, it does not include the 20 bp spacer/targeting sequence that is used to guide Cas9 to target DNA.

Extension arm—a single strand extension at the 3′ end or the 5′ end of the pegRNA which comprises a primer binding site and a DNA synthesis template sequence that encodes via a polymerase (e.g., a reverse transcriptase) a single stranded DNA flap containing the genetic change of interest, which then integrates into the endogenous DNA by replacing the corresponding endogenous strand, thereby installing the desired genetic change.

Transcription terminator—the guide RNA or pegRNA may comprise a transcriptional termination sequence at the 3′ of the molecule.

### Host Cell

The term “host cell,” as used herein, refers to a cell that can host, replicate, and express a vector described herein, e.g., a vector comprising a nucleic acid molecule encoding an MLH1 variant and a fusion protein comprising a Cas9 or Cas9 equivalent and a reverse transcriptase.

### Linker

The term “linker.” as used herein, refers to a molecule linking two other molecules or moieties. The linker can be an amino acid sequence in the case of a linker joining two fusion proteins. For example, a Cas9 can be fused to a reverse transcriptase by an amino acid linker sequence. The linker can also be a nucleotide sequence in the case of joining two nucleotide sequences together. For example, in the instant case, the traditional guide RNA is linked via a spacer or linker nucleotide sequence to the RNA extension of a prime editing guide RNA which may comprise a RT template sequence and an RT primer binding site. In other embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

napDNAbp

As used herein, the term “nucleic acid programmable DNA binding protein” or “napDNAbp,” of which Cas9 is an example, refer to proteins that use RNA:DNA hybridization to target and bind to specific sequences in a DNA molecule. Each napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp (e.g., Cas9 or equivalent) to localize and bind to a complementary sequence.

Without being bound by theory, the binding mechanism of a napDNAbp-guide RNA complex, in general, includes the step of forming an R-loop whereby the napDNAbp induces the unwinding of a double-strand DNA target, thereby separating the strands in the region bound by the napDNAbp. The guide RNA protospacer then hybridizes to the “target strand.” This displaces a “non-target strand” that is complementary to the target strand, which forms the single strand region of the R-loop. In some embodiments, the napDNAbp includes one or more nuclease activities, which then cut the DNA, leaving various types of lesions. For example, the napDNAbp may comprises a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand. Exemplary napDNAbp with different nuclease activities include “Cas9 nickase” (“nCas9”) and a deactivated Cas9 having no nuclease activities (“dead Cas9” or “dCas9”). Exemplary sequences for these and other napDNAbp are provided herein.

### Nickase

The term “nickase” refers to a Cas9 with one of the two nuclease domains inactivated. This enzyme is capable of cleaving only one strand of a target DNA.

### Non-Cognate Amino Acid

The term “non-cognate amino acid” refers to an amino acid that pairs with a tRNA molecule that does not comprise an anticodon sequence encoding said amino acid.

### Nonsense Mutation

The term “nonsense mutation” refers to a mutation in which a sense codon that corresponds to one of the twenty amino acids specified by the genetic code is changed to a chain-terminating codon (e.g., an opal stop codon, an amber stop codon, or a, ochre stop codon).

### Nonsense Suppressor Anticodon Sequence

The term “nonsense suppressor anticodon sequence” refers to an anticodon sequence that is complementary to an opal stop codon (e.g., 5′-UCA-3′), an amber codon (e.g., 5′-CUA-3′), or an ochre stop codon (e.g., 5′-UUA-3′).

### Nucleic Acid Molecule

The term “nucleic acid,” as used herein, refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5 bromouridine, C5 fluorouridine, C5 iodouridine, C5 propynyl uridine, C5 propynyl cytidine, C5 methylcytidine, 7 deazaadenosine, 7 deazaguanosine, 8 oxoadenosine, 8 oxoguanosine, 0(6) methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′ N phosphoramidite linkages).

### Nuclear Localization Sequence

The term “nuclear localization sequence” or “NLS” refers to an amino acid sequence that promotes import of a protein into the cell nucleus, for example, by nuclear transport.

pegRNA

As used herein, the terms “prime editing guide RNA” or “pegRNA” or “extended guide RNA” refer to a specialized form of a guide RNA that has been modified to include one or more additional sequences for implementing the prime editing methods and compositions described herein. As described herein, the prime editing guide RNA comprise one or more “extended regions” of nucleic acid sequence. The extended regions may comprise, but are not limited to, single-stranded RNA or DNA. Further, the extended regions may occur at the 3′ end of a traditional guide RNA. In other arrangements, the extended regions may occur at the 5′ end of a traditional guide RNA. In still other arrangements, the extended region may occur at an intramolecular region of the traditional guide RNA, for example, in the gRNA core region which associates and/or binds to the napDNAbp. The extended region comprises a “DNA synthesis template” which encodes (by the polymerase of the prime editor) a single-stranded DNA which, in turn, has been designed to be (a) homologous with the endogenous target DNA to be edited, and (b) which comprises at least one desired nucleotide change (e.g., a transition, a transversion, a deletion, or an insertion) to be introduced or integrated into the endogenous target DNA. The extended region may also comprise other functional sequence elements, such as, but not limited to, a “primer binding site” and a “spacer or linker” sequence, or other structural elements, such as, but not limited to aptamers, stem loops, hairpins, toe loops (e.g., a 3′ toe loop), or an RNA-protein recruitment domain (e.g., MS2 hairpin).

As used herein the “primer binding site” comprises a sequence that hybridizes to a single-strand DNA sequence having a 3′end generated from the nicked DNA of the R-loop.

In certain embodiments, the pegRNAs have a 5′ extension arm, a spacer, and a gRNA core. The 5′ extension further comprises in the 5′ to 3′ direction a reverse transcriptase template, a primer binding site, and a linker. The reverse transcriptase template may also be referred to more broadly as the “DNA synthesis template” where the polymerase of a prime editor described herein is not an RT, but another type of polymerase.

In certain other embodiments, the pegRNAs have a 5′ extension arm, a spacer, and a gRNA core. The 5′ extension further comprises in the 5′ to 3′ direction a reverse transcriptase template, a primer binding site, and a linker. The reverse transcriptase template may also be referred to more broadly as the “DNA synthesis template” where the polymerase of a prime editor described herein is not an RT, but another type of polymerase.

In still other embodiments, the pegRNAs have in the 5′ to 3′ direction a spacer (1), a gRNA core (2), and an extension arm (3). The extension arm (3) is at the 3′ end of the pegRNA. The extension arm (3) further comprises in the 5′ to 3′ direction a “primer binding site” (A), an “edit template” (B), and a “homology arm” (C). The extension arm (3) may also comprise an optional modifier region at the 3′ and 5′ ends, which may be the same sequences or different sequences. In addition, the 3′ end of the pegRNA may comprise a transcriptional terminator sequence. These sequence elements of the pegRNAs are further described and defined herein.

In still other embodiments, the pegRNAs have in the 5′ to 3′ direction an extension arm (3), a spacer (1), and a gRNA core (2). The extension arm (3) is at the 5′ end of the pegRNA. The extension arm (3) further comprises in the 3′ to 5′ direction a “primer binding site” (A), an “edit template” (B), and a “homology arm” (C). The extension arm (3) may also comprise an optional modifier region at the 3′ and 5′ ends, which may be the same sequences or different sequences. The pegRNAs may also comprise a transcriptional terminator sequence at the 3′ end. These sequence elements of the pegRNAs are further described and defined herein.

### PE1

As used herein, “PE1” refers to a PE complex comprising a fusion protein comprising Cas9(H840A) and a wild type MMLV RT having the following structure: [NLS]-[Cas9(H840A)]-[linker]-[MMLV_RT(wt)]+a desired pegRNA, wherein the PE fusion has the amino acid sequence of SEQ ID NO: 19, which is shown as follows;

(SEQ ID NO: 19)
MKRTADGSEFESPKKKRKVDKKYSIGLDIGTNSVGWAVITDEYKV
PSKKFKVLGNTDRHSIKKNLIGALLEDSGETAEATRLKRTARRRY
TRRKNRICYLQEIFSNEMAKVDDSFFHRLEESFLVEEDKKHERHP
IFGNIVDEVAYHEKYPTIYHLRKKLVDSTDKADLRLIYLALAHMI
KFRGHFLIEGDLNPDNSDVDKLFIQLVQTYNQLFEENPINASGVD
AKAILSARLSKSRRLENLIAQLPGEKKNGLFGNLIALSLGLTPNF
KSNFDLAEDAKLQLSKDTYDDDLDNLLAQIGDQYADLFLAAKNLS
DAILLSDILRYNTEITKAPLSASMIKRYDEHHQDLTLLKALVRQQ
LPEKYKEIFFDQSKNGYAGYIDGGASQEEFYKFIKPILEKMDGTE
ELLVKLNREDLLRKQRTEDNGSIPHQIHLGELHAILRRQEDFYPF
LKDNREKIEKILTFRIPYYVGPLARGNSRFAWMTRKSEETITPWN
FEEVVDKGASAQSFIERMTNFDKNLPNEKVLPKHSLLYEYFTVYN
ELTKVKYVTEGMRKPAFLSGEQKKAIVDLLFKTNRKVTVKQLKED
YFKKIECFDSVEISGVEDRFNASLGTYHDLLKIIKDKDFLDNEEN
EDILEDIVLTLTLFEDREMIEERLKTYAHLEDDKVMKQLKRRRYT
GWGRLSRKLINGIRDKQSGKTILDFLKSDGFANRNFMQLIHDDSL
TFKEDIQKAQVSGQGDSLHEHIANLAGSPAIKKGILQTVKVVDEL
VKVMGRHKPENIVIEMARENQTTQKGQKNSRERMKRIEEGIKELG
SQILKEHPVENTQLQNEKLYLYYLQNGRDMYVDQELDINRLSDYD
VDAIVPQSFLKDDSIDNKVLTRSDKNRGKSDNVPSEEVVKKMKNY
WRQLLNAKLITQRKFDNLTKAERGGLSELDKAGFIKRQLVETRQI
TKHVAQILDSRMNTKYDENDKLIREVKVITLKSKLVSDFRKDFQF
YKVREINNYHHAHDAYLNAVVGTALIKKYPKLESEFVYGDYKVYD
VRKMIAKSEQEIGKATAKYFFYSNIMNFFKTEITLANGEIRKRPL
IETNGETGEIVWDKGRDFATVRKVLSMPQVNIVKKTEVQTGGFSK
ESILPKRNSDKLIARKKDWDPKKYGGFDSPTVAYSVLVVAKVEKG
KSKKLKSVKELLGITIMERSSFEKNPIDFLEAKGYKEVKKDLIIK
LPKYSLFELENGRKRMLASAGELQKGNELALPSKYVNFLYLASHY
EKLKGSPEDNEQKQLFVEQHKHYLDEIIEQISEFSKRVILADANL
DKVLSAYNKHRDKPIREQAENIIHLFTLTNLGAPAAFKYEDTTID
RKRYTSTKEVLDATLIHQSITGLYETRIDLSQLGGDSGGSSGGSS
GSETPGTSESATPESSGGSSGGSSTLNIEDEYRLHETSKEPDVSL
GSTWLSDFPQAWAETGGMGLAVRQAPLIIPLKATSTPVSIKQYPM
SQEARLGIKPHIQRLLDQGILVPCQSPWNTPLLPVKKPGTNDYRP
VQDLREVNKRVEDIHPTVPNPYNLLSGLPPSHQWYTVLDLKDAFF
CLRLHPTSQPLFAFEWRDPEMGISGQLTWTRLPQGFKNSPTLFDE
ALHRDLADFRIQHPDLILLQYVDDLLLAATSELDCQQGTRALLQT
LGNLGYRASAKKAQICQKQVKYLGYLLKEGQRWLTEARKETVMGQ
PTPKTPRQLREFLGTAGFCRLWIPGFAEMAAPLYPLTKTGTLFNW
GPDQQKAYQEIKQALLTAPALGLPDLTKPFELFVDEKQGYAKGVL
TQKLGPWRRPVAYLSKKLDPVAAGWPPCLRMVAAIAVLTKDAGKL
TMGQPLVILAPHAVEALVKQPPDRWLSNARMTHYQALLLDTDRVQ
FGPVVALNPATLLPLPEEGLQHNCLDILAEAHGTRPDLTDQPLPD
ADHTWYTDGSSLLQEGQRKAGAAVTTETEVIWAKALPAGTSAQRA
ELIALTQALKMAEGKKLNVYTDSRYAFATAHIHGEIYRRRGLLTS
EGKEIKNKDEILALLKALFLPKRLSIIHCPGHQKGHSAEARGNRM
ADQAARKAAITETPDTSTLLIENSSPSGGSKRTADGSEFEPKKKR
KV
KEY:
NUCLEAR LOCALIZATION SEQUENCE (NLS)
TOP: (SEQ ID NO: 20),
BOTTOM: (SEQ ID NO: 21)
CAS9(H840A) (SEQ ID NO: 22)
33-AMINO ACID LINKER (SEQ ID NO: 23)
M-MLV reverse transcriptase (SEQ ID NO: 24).

### PE2

As used herein, “PE2” refers to a PE complex comprising a fusion protein comprising Cas9(H840A) and a variant MMLV RT having the following structure: [NLS]-[Cas9(H840A)]-[linker]-[MMLV_RT(D200N)(T330P)(L603W)(T306K)(W313F)]+a desired pegRNA, wherein the PE fusion has the amino acid sequence of SEQ ID NO: 25, which is shown as follows:

(SEQ ID NO: 25)
MKRTADGSEFESPKKKRKVDKKYSIGLDIGTNSVGWAVITDEYKVP
SKKFKVLGNTDRHSIKKNLIGALLFDSGETAEATRLKRTARRRYT
RRKNRICYLQEIFSNEMAKYDDSFFHRLEESELVEEDKKHERHPI
FGNIVDEVAYHEKYPTIYHLRKKLVDSTDKADLRLIYLALAHMIK
FRGHFLIEGDLNPDNSDVDKLFIQLVQTYNQLFEENPINASGVDA
KAILSARLSKSRRLENLIAQLPGEKKNGLFGNLIALSLGLTPNFK
SNEDLAEDAKLQLSKDTYDDDLDNLLAQIGDQYADLFLAAKNLSD
AILLSDILRVNTEITKAPLSASMIKRYDEHHQDLTLLKALVRQQL
PEKYKEIFFDQSKNGYAGYIDGGASQEEFYKFIKPILEKMDGTEE
LLVKLNREDLLRKQRTFDNGSIPHQIHLGELHAILRRQEDFYPFL
KDNREKIEKILTFRIPYYVGPLARGNSRFAWMTRKSEETITPWNF
EEVVDKGASAQSFIERMTNFDKNLPNEKVLPKHSLLYEYFTVYNE
LTKVKYVTEGMRKPAFLSGEQKKAIVDLLFKTNRKVTVKQLKEDY
FKKIECFDSVEISGVEDRENASLGTYHDLLKIIKDKDELDNEENE
DILEDIVLTLTLFEDREMIEERLKTYAHLEDDKVMKQLKRRRYTG
WGRLSRKLINGIRDKQSGKTILDELKSDGFANRNFMQLIHDDSLT
FKEDIQKAQVSGQGDSLHEHIANLAGSPAIKKGILQTVKVVDELV
KVMGRHKPENIVIEMARENQTTQKGQKNSRERMKRIEEGIKELGS
QILKEHPVENTQLQNEKLYLYYLQNGRDMYVDQELDINRLSDYDV
DAIVPQSFLKDDSIDNKVLTRSDKNRGKSDNVPSEEVVKKMKNYW
RQLLNAKLITQRKFDNLTKAERGGLSELDKAGFIKRQLVETRQIT
KHVAQILDSRMNTKYDENDKLIREVKVITLKSKLVSDFRKDFQFY
KVREINNYHHAHDAYLNAVVGTALIKKYPKLESEFVYGDYKVYDV
RKMIAKSEQEIGKATAKYFFYSNIMNFFKTEITLANGEIRKRPLI
ETNGETGEIVWDKGRDFATVRKVLSMPQVNIVKKTEVQTGGFSKE
SILPKRNSDKLIARKKDWDPKKYGGFDSPTVAYSVLVVAKVEKGK
SKKLKSVKELLGITIMERSSFEKNPIDFLEAKGYKEVKKDLIIKL
PKYSLFELENGRKRMLASAGELQKGNELALPSKYVNFLYLASHYE
KLKGSPEDNEQKQLFVEQHKHYLDEIIEQISEFSKRVILADANLD
KVLSAYNKHRDKPIREQAENIIHLFTLTNLGAPAAFKYFDTTIDR
KRYTSTKEVLDATLIHQSITGLYETRIDLSQLGGDSGGSSGGSSG
SETPGTSESATPESSGGSSGGSSTLNIEDEYRLHETSKEPDVSLG
STWLSDFPQAWAETGGMGLAVRQAPLIIPLKATSTPVSIKQYPMS
QEARLGIKPHIQRLLDQGILVPCQSPWNTPLLPVKKPGTNDYRPV
QDLREVNKRVEDIHPTVPNPYNLLSGLPPSHQWYTVLDLKDAFFC
LRLHPTSQPLFAFEWRDPEMGISGQLTWTRLPQGFKNSPTLFNEA
LHRDLADFRIQHPDLILLQYVDDLLLAATSELDCQQGTRALLQTL
GNLGYRASAKKAQICQKQVKYLGYLLKEGQRWLTEARKETVMGQP
TPKTPRQLREFLGKAGFCRLFIPGFAEMAAPLYPLTKPGTLFNWG
PDQQKAYQEIKQALLTAPALGLPDLTKPFELFVDEKQGYAKGVLT
QKLGPWRRPVAYLSKKLDPVAAGWPPCLRMVAAIAVLTKDAGKLT
MGQPLVILAPHAVEALVKQPPDRWLSNARMTHYQALLLDTDRVQF
GPVVALNPATLLPLPEEGLQHNCLDILAEAHGTRPDLTDQPLPDA
DHTWYTDGSSLLQEGQRKAGAAVTTETEVIWAKALPAGTSAQRAE
LIALTQALKMAEGKKLNVYTDSRYAFATAHIHGEIYRRRGWLTSE
GKEIKNKDEILALLKALFLPKRLSIIHCPGHQKGHSAEARGNRMA
DQAARKAAITETPDTSTLLIENSSPSGGSKRTADGSEFEPKKKRK
V
KEY:
NUCLEAR LOCALIZATION SEQUENCE (NLS)
TOP: (SEQ ID NO: 20),
BOTTOM: (SEQ ID NO: 21)
CAS9(H840A) (SEQ ID NO: 22)
33-AMINO ACID LINKER (SEQ ID NO: 23)
M-MLV reverse transcriptase (SEQ ID NO: 26).

### PE3

As used herein, “PE3” refers to PE2 plus a second-strand nicking guide RNA that complexes with the PE2 and introduces a nick in the non-edited DNA strand in order to induce preferential replacement of the edited strand.

### PE3b

As used herein, “PE3b” refers to PE3 but wherein the second-strand nicking guide RNA is designed for temporal control such that the second strand nick is not introduced until after the installation of the desired edit. This is achieved by designing a gRNA with a spacer sequence that matches only the edited strand, but not the original allele. Using this strategy, referred to hereafter as PE3b, mismatches between the protospacer and the unedited allele should disfavor nicking by the sgRNA until after the editing event on the PAM strand takes place.

### PE4

As used herein, “PE4” refers to a system comprising PE2 plus an MLH1 dominant negative protein (i.e., wild-type MLH1 with amino acids 754-756 truncated as described further herein) expressed in trans.

As used herein, “PE5” refers to a system comprising PE3 plus an MLH1 dominant negative protein (i.e., wild-type MLH1 with amino acids 754-756 truncated as described further herein, which may be referred to as “MLH1 Δ754-756” or “MLH1dn”) expressed in trans.

PE-shortAs used herein, “PE-short” refers to a PE construct that is fused to a C-terminally truncated reverse transcriptase, and has the following amino acid sequence:

(SEQ ID NO: 27)
MKRTADGSEFESPKKKRKVDKKYSIGLDIGTNSVGWAVITDEYKV
PSKKFKVLGNTDRHSIKKNLIGALLFDSGETAEATRLKRTARRRY
TRRKNRICYLQEIFSNEMAKVDDSFFHRLEESFLVEEDKKHERHP
IFGNIVDEVAYHEKYPTIYHLRKKLVDSTDKADLRLIYLALAHMI
KERGHFLIEGDLNPDNSDVDKLFIQLVQTYNQLFEENPINASGVD
AKAILSARLSKSRRLENLIAQLPGEKKNGLFGNLIALSLGLTPNF
KSNEDLAEDAKLQLSKDTYDDDLDNLLAQIGDQYADLFLAAKNLS
DAILLSDILRVNTEITKAPLSASMIKRYDEHHQDLTLLKALVRQQ
LPEKYKEIFFDQSKNGYAGYIDGGASQEEFYKFIKPILEKMDGTE
ELLVKLNREDLLRKQRTEDNGSIPHQIHLGELHAILRRQEDFYPF
LKDNREKIEKILTFRIPYYVGPLARGNSRFAWMTRKSEETITPWN
FEEVVDKGASAQSFIERMTNFDKNLPNEKVLPKHSLLYEYFTVYN
ELTKVKYVTEGMRKPAFLSGEQKKAIVDLLFKTNRKVTVKQLKED
YFKKIECFDSVEISGVEDRFNASLGTYHDLLKIIKDKDFLDNEEN
EDILEDIVLTLTLFEDREMIEERLKTYAHLEDDKVMKQLKRRRYT
GWGRLSRKLINGIRDKQSGKTILDFLKSDGFANRNFMQLIHDDSL
TFKEDIQKAQVSGQGDSLHEHIANLAGSPAIKKGILQTVKVVDEL
VKVMGRHKPENIVIEMARENQTTQKGQKNSRERMKRIEEGIKELG
SQILKEHPVENTQLQNEKLYLYYLQNGRDMYVDQELDINRLSDYD
VDAIVPQSFLKDDSIDNKVLTRSDKNRGKSDNVPSEEVVKKMKNY
WRQLLNAKLITQRKFDNLTKAERGGLSELDKAGFIKRQLVETRQI
TKHVAQILDSRMNTKYDENDKLIREVKVITLKSKLVSDFRKDFQF
YKVREINNYHHAHDAYLNAVVGTALIKKYPKLESEFVYGDYKVYD
VRKMIAKSEQEIGKATAKYFFYSNIMNFFKTEITLANGEIRKRPL
IETNGETGEIVWDKGRDFATVRKVLSMPQVNIVKKTEVQTGGFSK
ESILPKRNSDKLIARKKDWDPKKYGGFDSPTVAYSVLVVAKVEKG
KSKKLKSVKELLGITIMERSSFEKNPIDFLEAKGYKEVKKDLIIK
LPKYSLFELENGRKRMLASAGELQKGNELALPSKYVNFLYLASHY
EKLKGSPEDNEQKQLFVEQHKHYLDEIIEQISEFSKRVILADANL
DKVLSAYNKHRDKPIREQAENIIHLFTLTNLGAPAAFKYEDTTID
RKRYTSTKEVLDATLIHQSITGLYETRIDLSQLGGDSGGSSGGSS
GSETPGTSESATPESSGGSSGGSSTLNIEDEYRLHETSKEPDVSL
GSTWLSDFPQAWAETGGMGLAVRQAPLIIPLKATSTPVSIKQYPM
SQEARLGIKPHIQRLLDQGILVPCQSPWNTPLLPVKKPGTNDYRP
VQDLREVNKRVEDIHPTVPNPYNLLSGLPPSHQWYTVLDLKDAFF
CLRLHPTSQPLFAFEWRDPEMGISGQLTWTRLPQGFKNSPTLFNE
ALHRDLADFRIQHPDLILLQYVDDLLLAATSELDCQQGTRALLQT
LGNLGYRASAKKAQICQKQVKYLGYLLKEGQRWLTEARKETVMGQ
PTPKTPRQLREFLGKAGFCRLFIPGFAEMAAPLYPLTKPGTLFNW
GPDQQKAYQEIKQALLTAPALGLPDLTKPFELFVDEKQGYAKGVL
TQKLGPWRRPVAYLSKKLDPVAAGWPPCLRMVAAIAVLTKDAGKL
TMGQPLVILAPHAVEALVKQPPDRWLSNARMTHYQALLLDTDRVQ
FGPVVALNPATLLPLPEEGLQHNCLDNSRLINSGGSKRTADGSEF
EPKKKRKV
KEY:
NUCLEAR LOCALIZATION SEQUENCE (NLS)
TOP: (SEQ ID NO: 20),
BOTTOM: (SEQ ID NO: 21)
CAS9(H840A) (SEQ ID NO: 22)
33-AMINO ACID LINKER 1 (SEQ ID NO: 23)
M-MLV TRUNCATED REVERSE TRANSCRIPTASE
(SEQ ID NO: 28)

### Polymerase

As used herein, the term “polymerase” refers to an enzyme that synthesizes a nucleotide strand and that may be used in connection with the prime editor systems described herein. The polymerase can be a “template-dependent” polymerase (i.e., a polymerase that synthesizes a nucleotide strand based on the order of nucleotide bases of a template strand). The polymerase can also be a “template-independent” polymerase (i.e., a polymerase that synthesizes a nucleotide strand without the requirement of a template strand). A polymerase may also be further categorized as a “DNA polymerase” or an “RNA polymerase.” In various embodiments, the prime editor system comprises a DNA polymerase. In various embodiments, the DNA polymerase can be a “DNA-dependent DNA polymerase” (i.e., whereby the template molecule is a strand of DNA). In such cases, the DNA template molecule can be a pegRNA, wherein the extension arm comprises a strand of DNA. In such cases, the pegRNA may be referred to as a chimeric or hybrid pegRNA which comprises an RNA portion (i.e., the guide RNA components, including the spacer and the gRNA core) and a DNA portion (i.e., the extension arm). In various other embodiments, the DNA polymerase can be an “RNA-dependent DNA polymerase” (i.e., whereby the template molecule is a strand of RNA). In such cases, the pegRNA is RNA, i.e., including an RNA extension. The term “polymerase” may also refer to an enzyme that catalyzes the polymerization of nucleotide (i.e., the polymerase activity). Generally, the enzyme will initiate synthesis at the 3′-end of a primer annealed to a polynucleotide template sequence (e.g., such as a primer sequence annealed to the primer binding site of a pegRNA) and will proceed toward the 5′ end of the template strand. A “DNA polymerase” catalyzes the polymerization of deoxynucleotides. As used herein in reference to a DNA polymerase, the term DNA polymerase includes a “functional fragment thereof”. A “functional fragment thereof” refers to any portion of a wild-type or mutant DNA polymerase that encompasses less than the entire amino acid sequence of the polymerase and which retains the ability, under at least one set of conditions, to catalyze the polymerization of a polynucleotide. Such a functional fragment may exist as a separate entity, or it may be a constituent of a larger polypeptide, such as a fusion protein.

### Premature Termination Stop Codon

The term “premature termination stop codon” or “PTC” refers to a nonsense mutation in a DNA sequence encoding an mRNA sequence and/or in the mRNA sequence, wherein the stop codon occurs earlier in the sequence, relative to the non-mutated mRNA sequence, and thus impedes translation of the full-length protein encoded by the mRNA sequence leading to a truncated protein. Premature termination codon may be an ochre stop codon comprising a 5′-UAA-3′ codon sequence, an opal stop codon comprising a 5′-UGA-3′ codon sequence, or an amber stop codon comprising a 5′-UAG-3′ codon sequence.

### Prime Editing

As used herein, the term “prime editing” refers to an approach for gene editing using napDNAbps, a polymerase (e.g., a reverse transcriptase), and specialized guide RNAs that include a DNA synthesis template for encoding desired new genetic information (or deleting genetic information) that is then incorporated into a target DNA sequence. Classical prime editing is described in the inventors publication of Anzalone, A. V. et al. Search-and-replace genome editing without double-strand breaks or donor DNA. Nature 576, 149-157 (2019), which is incorporated herein by reference in its entirety.

Prime editing represents a platform for genome editing that is a versatile and precise genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“pegRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same (or is homologous to) sequence as the endogenous strand (immediately downstream of the nick site) of the target site to be edited (with the exception that it includes the desired edit). Through DNA repair and/or replication machinery, the endogenous strand downstream of the nick site is replaced by the newly synthesized replacement strand containing the desired edit. In some cases, prime editing may be thought of as a “search-and-replace” genome editing technology since the prime editors, as described herein, not only search and locate the desired target site to be edited, but at the same time, encode a replacement strand containing a desired edit which is installed in place of the corresponding target site endogenous DNA strand. The prime editors of the present disclosure relate, in part, to the discovery that the mechanism of target-primed reverse transcription (TPRT) or “prime editing” can be leveraged or adapted for conducting precision CRISPR/Cas-based genome editing with high efficiency and genetic flexibility. TPRT is naturally used by mobile DNA elements, such as mammalian non-LTR retrotransposons and bacterial Group II introns. The inventors have herein used Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA. However, while the concept begins with prime editors that use reverse transcriptase as the DNA polymerase component, the prime editors described herein are not limited to reverse transcriptases but may include the use of virtually any DNA polymerase. Indeed, while the application throughout may refer to prime editors with “reverse transcriptases,” it is set forth here that reverse transcriptases are only one type of DNA polymerase that may work with prime editing. Thus, where ever the specification mentions a “reverse transcriptase,” the person having ordinary skill in the art should appreciate that any suitable DNA polymerase may be used in place of the reverse transcriptase. Thus, in one aspect, the prime editors may comprise Cas9 (or an equivalent napDNAbp) which is programmed to target a DNA sequence by associating it with a specialized guide RNA (i.e., pegRNA) containing a spacer sequence that anneals to a complementary protospacer in the target DNA. The specialized guide RNA also contains new genetic information in the form of an extension that encodes a replacement strand of DNA containing a desired genetic alteration which is used to replace a corresponding endogenous DNA strand at the target site. To transfer information from the pegRNA to the target DNA, the mechanism of prime editing involves nicking the target site in one strand of the DNA to expose a 3′-hydroxyl group. The exposed 3′-hydroxyl group can then be used to prime the DNA polymerization of the edit-encoding extension on pegRNA directly into the target site. In various embodiments, the extension—which provides the template for polymerization of the replacement strand containing the edit—can be formed from RNA or DNA. In the case of an RNA extension, the polymerase of the prime editor can be an RNA-dependent DNA polymerase (such as, a reverse transcriptase). In the case of a DNA extension, the polymerase of the prime editor may be a DNA-dependent DNA polymerase. The newly synthesized strand (i.e., the replacement DNA strand containing the desired edit) that is formed by the herein disclosed prime editors would be homologous to the genomic target sequence (i.e., have the same sequence as) except for the inclusion of a desired nucleotide change (e.g., a single nucleotide change, a deletion, or an insertion, or a combination thereof). The newly synthesized (or replacement) strand of DNA may also be referred to as a single strand DNA flap, which would compete for hybridization with the complementary homologous endogenous DNA strand, thereby displacing the corresponding endogenous strand. In certain embodiments, the system can be combined with the use of an error-prone reverse transcriptase enzyme (e.g., provided as a fusion protein with the Cas9 domain, or provided in trans to the Cas9 domain). The error-prone reverse transcriptase enzyme can introduce alterations during synthesis of the single strand DNA flap. Thus, in certain embodiments, error-prone reverse transcriptase can be utilized to introduce nucleotide changes to the target DNA. Depending on the error-prone reverse transcriptase that is used with the system, the changes can be random or non-random. Resolution of the hybridized intermediate (comprising the single strand DNA flap synthesized by the reverse transcriptase hybridized to the endogenous DNA strand) can include removal of the resulting displaced flap of endogenous DNA (e.g., with a 5′ end DNA flap endonuclease, FEN1), ligation of the synthesized single strand DNA flap to the target DNA, and assimilation of the desired nucleotide change as a result of cellular DNA repair and/or replication processes. Because templated DNA synthesis offers single nucleotide precision for the modification of any nucleotide, including insertions and deletions, the scope of this approach is very broad and could foreseeably be used for myriad applications in basic science and therapeutics.

In various embodiments, prime editing operates by contacting a target DNA molecule (for which a change in the nucleotide sequence is desired to be introduced) with a nucleic acid programmable DNA binding protein (napDNAbp) complexed with a prime editing guide RNA (pegRNA). In various embodiments, the prime editing guide RNA (pegRNA) comprises an extension at the 3′ or 5′ end of the guide RNA, or at an intramolecular location in the guide RNA and encodes the desired nucleotide change (e.g., single nucleotide change, insertion, or deletion). In step (a), the napDNAbp/extended gRNA complex contacts the DNA molecule and the extended gRNA guides the napDNAbp to bind to a target locus. In step (b), a nick in one of the strands of DNA of the target locus is introduced (e.g., by a nuclease or chemical agent), thereby creating an available 3′ end in one of the strands of the target locus. In certain embodiments, the nick is created in the strand of DNA that corresponds to the R-loop strand, i.e., the strand that is not hybridized to the guide RNA sequence, i.e., the “non-target strand.” The nick, however, could be introduced in either of the strands. That is, the nick could be introduced into the R-loop “target strand” (i.e., the strand hybridized to the protospacer of the extended gRNA) or the “non-target strand” (i.e., the strand forming the single-stranded portion of the R-loop and which is complementary to the target strand). In step (c), the 3′ end of the DNA strand (formed by the nick) interacts with the extended portion of the guide RNA in order to prime reverse transcription (i.e., “target-primed RT”). In certain embodiments, the 3′ end DNA strand hybridizes to a specific RT priming sequence on the extended portion of the guide RNA, i.e., the “reverse transcriptase priming sequence” or “primer binding site” on the pegRNA. In step (d), a reverse transcriptase (or other suitable DNA polymerase) is introduced which synthesizes a single strand of DNA from the 3′ end of the primed site towards the 5′ end of the prime editing guide RNA. The DNA polymerase (e.g., reverse transcriptase) can be fused to the napDNAbp or alternatively can be provided in trans to the napDNAbp. This forms a single-strand DNA flap comprising the desired nucleotide change (e.g., the single base change, insertion, or deletion, or a combination thereof) and which is otherwise homologous to the endogenous DNA at or adjacent to the nick site. In step (e), the napDNAbp and guide RNA are released. Steps (f) and (g) relate to the resolution of the single strand DNA flap such that the desired nucleotide change becomes incorporated into the target locus. This process can be driven towards the desired product formation by removing the corresponding 5′ endogenous DNA flap that forms once the 3′ single strand DNA flap invades and hybridizes to the endogenous DNA sequence. Without being bound by theory, the cells endogenous DNA repair and replication processes resolves the mismatched DNA to incorporate the nucleotide change(s) to form the desired altered product. The process can also be driven towards product formation with “second strand nicking.” This process may introduce at least one or more of the following genetic changes: transversions, transitions, deletions, and insertions.

The term “prime editor (PE) system” or “prime editor (PE)” or “PE system” or “PE editing system” refers the compositions involved in the method of genome editing using target-primed reverse transcription (TPRT) describe herein, including, but not limited to the napDNAbps, reverse transcriptases, fusion proteins (e.g., comprising napDNAbps and reverse transcriptases), prime editing guide RNAs, and complexes comprising fusion proteins and prime editing guide RNAs, as well as accessory elements, such as second strand nicking components (e.g., second strand sgRNAs) and 5′ endogenous DNA flap removal endonucleases (e.g., FEN1) for helping to drive the prime editing process towards the edited product formation.

Although in the embodiments described thus far the pegRNA constitutes a single molecule comprising a guide RNA (which itself comprises a spacer sequence and a gRNA core or scaffold) and a 5′ or 3′ extension arm comprising the primer binding site and a DNA synthesis template, the pegRNA may also take the form of two individual molecules comprised of a guide RNA and a trans prime editor RNA template (tPERT), which essentially houses the extension arm (including, in particular, the primer binding site and the DNA synthesis domain) and an RNA-protein recruitment domain (e.g., MS2 aptamer or hairpin) in the same molecule which becomes co-localized or recruited to a modified prime editor complex that comprises a tPERT recruiting protein (e.g., MS2cp protein, which binds to the MS2 aptamer).

### Prime Editor

The term “prime editor” or refers to fusion constructs comprising a napDNAbp (e.g., Cas9 nickase) and a reverse transcriptase that is capable of carrying out prime editing on a target nucleotide sequence in the presence of a pegRNA (or “extended guide RNA”).

### Prime Editor Complex

The term “prime editor complex” refers to The term “prime editor” may refer to the fusion protein or to the fusion protein complexed with a pegRNA, and/or further complexed with a second-strand nicking sgRNA. In some embodiments, the prime editor may also refer to the complex comprising a fusion protein (reverse transcriptase fused to a napDNAbp), a pegRNA, and a regular guide RNA capable of directing the second-site nicking step of the non-edited strand as described herein.

### Primer Binding Site

The term “primer binding site” or “the PBS” refers to the nucleotide sequence located on a pegRNA as a component of the extension arm (typically at the 3′ end of the extension arm) and serves to bind to the primer sequence that is formed after Cas9 nicking of the target sequence by the prime editor. As detailed elsewhere, when the Cas9 nickase component of a prime editor nicks one strand of the target DNA sequence, a 3′-ended ssDNA flap is formed, which serves a primer sequence that anneals to the primer binding site on the pegRNA to prime reverse transcription.

### Protospacer

As used herein, the term “protospacer” refers to the sequence (˜20 bp) in DNA adjacent to the PAM (protospacer adjacent motif) sequence. The protospacer shares the same sequence as the spacer sequence of the guide RNA. The guide RNA anneals to the complement of the protospacer sequence on the target DNA (specifically, one strand thereof, i.e., the “target strand” versus the “non-target strand” of the target DNA sequence). In order for Cas9 to function it also requires a specific protospacer adjacent motif (PAM) that varies depending on the bacterial species of the Cas9 gene. The most commonly used Cas9 nuclease, derived from S. pyogenes, recognizes a PAM sequence of NGG that is found directly downstream of the target sequence in the genomic DNA, on the non-target strand. The skilled person will appreciate that the literature in the state of the art sometimes refers to the “protospacer” as the ˜20-nt target-specific guide sequence on the guide RNA itself, rather than referring to it as a “spacer.” Thus, in some cases, the term “protospacer” as used herein may be used interchangeably with the term “spacer.” The context of the description surrounding the appearance of either “protospacer” or “spacer” will help inform the reader as to whether the term is in reference to the gRNA or the DNA target.

### Redundant and Dispensable DNA Sequence

As used herein, the term “redundant and dispensable DNA sequence” refers to a DNA sequence encoding a tRNA gene that has codon degeneracy. Codon degeneracy means that there is more than one codon, and hence anticodon, that specifies a single amino acid.

### Protospacer Adjacent Motif (PAM)

As used herein, the term “protospacer adjacent sequence” or “PAM” refers to an approximately 2-6 base pair DNA sequence that is an important targeting component of a Cas9 nuclease. Typically, the PAM sequence is on either strand, and is downstream in the 5′ to 3′ direction of the Cas9 cut site. The canonical PAM sequence (i.e., the PAM sequence that is associated with the Cas9 nuclease of Streptococcus pyogenes or SpCas9) is 5′-NGG-3′ wherein “N” is any nucleobase followed by two guanine (“G”) nucleobases. Different PAM sequences can be associated with different Cas9 nucleases or equivalent proteins from different organisms. In addition, any given Cas9 nuclease, e.g., SpCas9, may be modified to alter the PAM specificity of the nuclease such that the nuclease recognizes alternative PAM sequence.

For example, with reference to the canonical SpCas9 amino acid sequence is SEQ ID NO: 18, the PAM sequence can be modified by introducing one or more mutations, including (a) D1135V, R1335Q, and T1337R “the VQR variant”, which alters the PAM specificity to NGAN or NGNG, (b) D1135E, R1335Q, and T1337R “the EQR variant”, which alters the PAM specificity to NGAG, and (c) D1135V, G1218R, R1335E, and T1337R “the VRER variant”, which alters the PAM specificity to NGCG. In addition, the D1135E variant of canonical SpCas9 still recognizes NGG, but it is more selective compared to the wild type SpCas9 protein.

It will also be appreciated that Cas9 enzymes from different bacterial species (i.e., Cas9 orthologs) can have varying PAM specificities. For example, Cas9 from Staphylococcus aureus (SaCas9) recognizes NGRRT or NGRRN. In addition, Cas9 from Neisseria meningitis (NmCas) recognizes NNNNGATT. In another example, Cas9 from Streptococcus thermophilis (StCas9) recognizes NNAGAAW. In still another example, Cas9 from Treponema denticola (TdCas) recognizes NAAAAC. These are examples and are not meant to be limiting. It will be further appreciated that non-SpCas9s bind a variety of PAM sequences, which makes them useful when no suitable SpCas9 PAM sequence is present at the desired target cut site. Furthermore, non-SpCas9s may have other characteristics that make them more useful than SpCas9. For example, Cas9 from Staphylococcus aureus (SaCas9) is about 1 kilobase smaller than SpCas9, so it can be packaged into adeno-associated virus (AAV). Further reference may be made to Shah et al., “Protospacer recognition motifs: mixed identities and functional diversity,” RNA Biology, 10(5): 891-899 (which is incorporated herein by reference).

### Recombinase

The term “recombinase” refers to any enzyme that catalyzes site-specific recombination events within DNA. In some embodiments, the recombinase is a site-specific recombinase (SSRs). SSRs refer to any enzyme capable of rearranging DNA segments by recognizing and binding to short specific DNA sequences, at which they cleave the DNA backbone, exchange the two DNA helices involved, and rejoin the DNA strands. In some embodiments, the recombinase comprises an integrase (e.g., a serine integrase such as Bxb1). In some embodiments, the recombinase binds to a recognition site and cleaves the DNA at the recognition site.

### Reverse Transcriptase

The term “reverse transcriptase” describes a class of polymerases characterized as RNA-dependent DNA polymerases. All known reverse transcriptases require a primer to synthesize a DNA transcript from an RNA template. Historically, reverse transcriptase has been used primarily to transcribe mRNA into cDNA which can then be cloned into a vector for further manipulation. Avian myoblastosis virus (AMV) reverse transcriptase was the first widely used RNA-dependent DNA polymerase (Verma, Biochim. Biophys. Acta 473:1 (1977)). The enzyme has 5′-3′ RNA-directed DNA polymerase activity, 5′-3′ DNA-directed DNA polymerase activity, and RNase H activity. RNase H is a processive 5′ and 3′ ribonuclease specific for the RNA strand for RNA-DNA hybrids (Perbal, A Practical Guide to Molecular Cloning, New York: Wiley & Sons (1984)). Errors in transcription cannot be corrected by reverse transcriptase because known viral reverse transcriptases lack the 3′-5′ exonuclease activity necessary for proofreading (Saunders and Saunders, Microbial Genetics Applied to Biotechnology, London: Croom Helm (1987)). A detailed study of the activity of AMV reverse transcriptase and its associated RNase H activity has been presented by Berger et al., Biochemistry 22:2365-2372 (1983). Another reverse transcriptase which is used extensively in molecular biology is reverse transcriptase originating from Moloney murine leukemia virus (M-MLV). See, e.g., Gerard, G. R., DNA 5:271-279 (1986), and Kotewicz, M. L., et al., Gene 35:249-258 (1985). M-MLV reverse transcriptase substantially lacking in RNase H activity has also been described. See, e.g., U.S. Pat. No. 5,244,797. The invention contemplates the use of any such reverse transcriptases, or variants or mutants thereof.

In addition, the invention contemplates the use of reverse transcriptases that are error-prone, i.e., that may be referred to as error-prone reverse transcriptases or reverse transcriptases that do not support high fidelity incorporation of nucleotides during polymerization. During synthesis of the single-strand DNA flap based on the RT template integrated with the guide RNA, the error-prone reverse transcriptase can introduce one or more nucleotides which are mismatched with the RT template sequence, thereby introducing changes to the nucleotide sequence through erroneous polymerization of the single-strand DNA flap. These errors introduced during synthesis of the single strand DNA flap then become integrated into the double strand molecule through hybridization to the corresponding endogenous target strand, removal of the endogenous displaced strand, ligation, and then through one more round of endogenous DNA repair and/or sequencing processes.

### Reverse Transcription

As used herein, the term “reverse transcription” indicates the capability of an enzyme to synthesize a DNA strand (that is, complementary DNA or cDNA) using RNA as a template. In some embodiments, the reverse transcription can be “error-prone reverse transcription,” which refers to the properties of certain reverse transcriptase enzymes which are error-prone in their DNA polymerization activity.

### Pharmaceutically Acceptable Carrier

As used here, the term “pharmaceutically acceptable carrier” means a pharmaceutically-acceptable material, composition or vehicle, such as a liquid or solid filler, diluent, excipient, manufacturing aid (e.g., lubricant, tale magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.).

### Protein, Peptide, and Polypeptide

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

### Safe Harbor Locus Site

The term “safe harbor locus site” refers to any site in the genome able to accommodate the integration of new genetic material in a manner that ensures that the newly inserted genetic elements (i) function predictably and (ii) do not cause alternations of the host genome posing a risk to the host cell or organism. Exemplary embodiments include, but are not limited to, ROSA26, CCR5, and AAVS1.

### Spacer Sequence

As used herein, the term “spacer sequence” in connection with a guide RNA or a pegRNA refers to the portion of the guide RNA or pegRNA of about 20 nucleotides which contains a nucleotide sequence that shares the same sequence as the protospacer sequence in the target DNA sequence. The spacer sequence anneals to the complement of the protospacer sequence to form a ssRNA/ssDNA hybrid structure at the target site and a corresponding R loop ssDNA structure of the endogenous DNA strand.

Suppressor tRNA

The term “suppressor tRNA” refers to a tRNA (defined elsewhere herein) charged with an amino acid comprising a mutation in the anticodon that allows it to recognize a premature stop codon (defined elsewhere herein as either an amber, ochre, or opal stop codon) on an mRNA and to and insert an amino acid into the amino acid sequence encoded by the mRNA, thus preventing truncation of the amino acid sequence.

### Target Site

The term “target site” refers to a sequence within a nucleic acid molecule that is edited by a prime editor (PE) disclosed herein. The target site further refers to the sequence within a nucleic acid molecule to which a complex of the prime editor (PE) and gRNA binds.

tRNA

The terms “tRNA” or “endogenous tRNA” or “unedited tRNA” collectively refer to a transfer RNA as found in nature. tRNA is an art recognized term that refers to a molecule composed of RNA that serves as the physical link between mRNA and the amino acid sequence of proteins. The tRNA structure consists of the following: (i) a 5′-terminal phosphate group, (ii) an acceptor stem made by the base pairing of the 5′-terminal new nucleotide with the 3′-terminal nucleotide (which contains the CCA 3′-terminal group used to attach the amino acid), (iii) a CCA tail at the 3′-end of the tRNA molecule that is covalently bound to an amino acid (herein “aminoacyl-tRNA), (iv) a D arm domain, (v) an anticodon arm comprising an anticodon sequence. The tRNA 5′-to-3′ primary structure contains the anticodon but in reverse order, since 3′-to-5′ directionality is required to read the mRNA from 5′-to-3′, (vi) a T arm domain, and (vii) a variable arm domain

### Variant

As used herein the term “variant” should be taken to mean the exhibition of qualities that have a pattern that deviates from what occurs in nature, e.g., a variant Cas9 is a Cas9 comprising one or more changes in amino acid residues as compared to a wild type Cas9 amino acid sequence. The term “variant” encompasses homologous proteins having at least 75%, or at least 80%, or at least 85%, or at least 90%, or at least 95%, or at least 99% percent identity with a reference sequence and having the same or substantially the same functional activity or activities as the reference sequence. The term also encompasses mutants, truncations, or domains of a reference sequence, and which display the same or substantially the same functional activity or activities as the reference sequence.

### Vector

The term “vector,” as used herein, refers to a nucleic acid that can be modified to encode a gene of interest and that is able to enter into a host cell, mutate and replicate within the host cell, and then transfer a replicated form of the vector into another host cell. Exemplary suitable vectors include viral vectors, such as retroviral vectors or bacteriophages and filamentous phage, and conjugative plasmids. Additional suitable vectors will be apparent to those of skill in the art based on the instant disclosure.

### DETAILED DESCRIPTION

Aspects of the disclosure relate to methods, compositions, and systems for editing an endogenous tRNA into a suppressor tRNA using prime editing (e.g., to treat diseases caused by premature termination codons). Other aspects of the disclosure relate to methods, compositions, and systems for editing an endogenous indispensable tRNA into a suppressor tRNA using prime editing (e.g., to treat diseases caused by premature termination codons). Additional aspects relate to compositions comprising the prime editing machinery (e.g., fusion protein comprising a nucleic acid programmable DNA binding protein and reverse transcriptase and/or pegRNA, etc.) and/or complexes comprising the prime editor and pegRNA that are capable of editing an endogenous tRNA into a suppressor tRNA. In some aspects, the disclosure further relates to polynucleotides encoding one or more nucleic acid sequences encoding the prime editor and/or pegRNA, cells comprising the polynucleotides and complexes comprising the prime editor and pegRNA, kits comprising any one of the compositions, complexes, polynucleotides, vectors (e.g., AAV), and/or cells disclosed herein, and/or delivery systems for administering any one of the compositions, complexes, polynucleotides, vectors to a subject in need thereof (e.g., lipid nanoparticles). Additional aspects relate to methods for inserting a new suppressor tRNA gene into a target site in a genome (e.g., a safe harbor locus site) using prime editing.

Installing Suppressor tRNAs Via Prime Editing Endogenous tRNAs

Aspects of the disclosure relate to methods for editing a DNA sequence encoding a tRNA at a target site. The target site in the DNA sequence, according to some embodiments, encodes for one or more domains of the tRNA. In some embodiments, the domain is a D-arm domain, a T-arm domain, a variable arm domain, an acceptor stem domain, and an anticodon arm domain comprising an anticodon sequence.

As used herein, the term “D-arm domain” refers to a feature in the tertiary structure of tRNA. Without wishing to be bound by theory, it comprises two D stems and the D loop. The D loop further comprises the base dihydrouridine, for which the arm is named. The D-loops main function is recognition. It is widely believed that it acts as a recognition site for aminoacyl-tRNA synthetase, an enzyme involved in the aminoacylation of the tRNA molecule.

As used herein, the term “T-arm domain” refers to a specialized region of the tRNA which acts as a special recognition site for the ribosome to form a tRNA-ribosome complex during protein biosynthesis (e.g., translation). The T-arm domain is generally believed to have two components: a T-stem and T-loop. There are two T-stems of five base pairs each. The T-loop is often referred to as the TψC arm due to the presence of thymidine, pseudouridine and cytidine.

As used herein, the term “anticodon arm domain” refers to a 5-bp stem whose loop contains the anticodon. The anticodon portion of the tRNA binds to the codon sequence in mRNA during translation.

As used herein, the term “variable arm domain” refers to a loop that present between the anticodon arm and the TψC arm. The length of the variable arm domain is important in the recognition of the aminoacyl-tRNA synthetase for the tRNA. In some embodiments, the tRNA lacks the variable arm domain.

In some embodiments, the methods comprise editing a DNA sequence encoding an endogenous tRNA at a target site, comprising contacting the DNA sequence at the target site with a prime editor and a pegRNA, wherein the prime editor installs one or more modifications in the DNA sequence at the target site, relative to the DNA sequence encoding the endogenous tRNA, thus converting the encoded tRNA into an encoded suppressor tRNA, wherein the pegRNA comprises a spacer sequence, a gRNA core, and an extension arm

In some embodiments, the methods comprise editing a editing a DNA sequence encoding an endogenous tRNA at a target site, comprising contacting the DNA sequence at the target site with a prime editor and a pegRNA, wherein the prime editor installs one or more modifications in the DNA sequence at the target site, relative to the DNA sequence encoding the endogenous tRNA, thus converting the encoded tRNA into an encoded suppressor tRNA, wherein the pegRNA comprises a spacer sequence, a gRNA core, and an extension arm, wherein the spacer sequence and extension arms are any sequences listed in Table 2, wherein the DNA sequence is any sequence listed in Table 1.

In some embodiments, the methods comprise contacting the DNA sequence at a target site with a prime editor and a pegRNA. The prime editor may install one or more modifications at the target site (e.g., insertion, deletion, or substitution), relative to the endogenous tRNA, thus converting said tRNA into a suppressor tRNA. In some embodiments, the one or more modifications comprise installing a single base nucleotide in the variable arm domain of the tRNA. In some embodiments, installing the single base nucleotide in the variable arm results in replacement of a cognate amino acid with a non-cognate amino acid. In some embodiments, the non-cognate amino acid is serine.

In some embodiments, the one or more edits (e.g., modifications) are selected from the group consisting of insertions, deletions, and substitution. In some embodiments, the edit is an insertion. In some embodiments, the edits are deletions. In some embodiments, the edits are substitutions.

In some embodiments, the one or more modifications comprise installing a C70U mutation in the acceptor stem domain. In some embodiments, installing the C70U mutation creates a G3:U70 base pair in the acceptor stem domain and results in the replacement of the cognate amino acid with a non-cognate amino acid. In some embodiments, the non-cognate amino acid is alanine.

In some embodiments, the one or more modifications comprise installing one or more edits (e.g., insertions, deletion, substitution, etc.) in the anticodon sequence of the anticodon arm domain, thus converting the anticodon sequence into a nonsense suppressor anticodon sequence. In some embodiments, the one or more modifications comprises substituting the DNA sequence encoding the anticodon sequence with a nonsense suppressor anticodon sequence. The nonsense suppressor sequence, in some embodiments, is selected from the group consisting of 5′-UUA-3′, 5′-UCA-3′, and 5′-CUA-3′.

In some embodiments, an edited tRNA comprising a nonsense suppressor anticodon is configured to bind to a PTC sequence. In some embodiments, the PTC is an ochre stop codon with sequence 5′-UAA-3′. In some embodiments, the PTC is an opal stop codon with sequence 5′-UGA-3′. In some embodiments, the PTC is an amber stop codon with sequence 5′-UAG-3′.

In some embodiments, the anticodon sequence is a single transition mutation away from a nonsense suppressor anticodon. As defined elsewhere herein, a nonsense suppressor anticodon is the complementary sequence to a premature termination codon or PTC. There are currently 3 known PTCs, each of which, comprises a different sequence. The ochre stop codon has sequence 5′ UAA 3′ and corresponds to nonsense suppressor anticodon with sequence 5′-UUA-3′. The opal stop codon has sequence 5′ UGA 3′ and corresponds to the nonsense suppressor anticodon with sequence 5′-UCA-3′. The amber stop codon has sequence 5′ UAG 3 and corresponds to nonsense suppressor anticodon with sequence 5′-CUA-3′.

The single transition mutation may be any transition mutation known in the art. For example, in some embodiments, the single transition mutation consists of a C>T (e.g., C-to-T) mutation, a T>C mutation (e.g., T-to-C) mutation, an A>G (e.g., A-to-G) mutation, or a G>A (G-to-A) mutation.

In some embodiments, the anticodon sequence is a single transversion mutation away from a nonsense suppressor anticodon. The single transversion mutation may be any transversion mutation known in the art. For example, in some embodiments, the single transversion mutation is selected from the group consisting of an A>C (e.g., A-to-C) mutation, T>G (T-to-G) mutation, G>T (G-to-T) mutation, C>A (C-to-A) mutation, C>G (C-to-G) mutation, G>C (G-to-C) mutation, A>T (A-to-T) mutation, and T>A (T-to-A) mutation.

In some embodiments, the endogenous tRNA comprises an anticodon sequence that is 3′-X1-X2-X3-5′. In some embodiments, the prime editor installs the mutation (e.g., transition or transversion) at position X1. In some embodiments, the mutation is selected from the group consisting of G>A, C>A, and U>A, relative to the endogenous tRNA. In some embodiments, the anticodon sequence comprises a N>A mutation at X1, C at X2, and U at X3, wherein N is G, C, or U (e.g., which is configured to bind to the PTC 5′-UGA-3′). In some embodiments, the anticodon sequence comprises a N>A mutation at X1, U at X2, and C at X3, wherein N is G, C, or U (e.g., which is configured to bind to the FTC 5′-UAG-3′). In some embodiments, the anticodon sequence comprises a N>A mutation at X1, U at X2, and U at X3, wherein N is G, C, or U (e.g., which is configured to bind to the PTC 5′-UAA-3′).

In some embodiments, the prime editor installs the mutation (e.g., transition or transversion) at position X2. In some embodiments, the mutation is selected from the group consisting of A>C, G>C, and U>C, relative to the endogenous tRNA. In some embodiments, the anticodon sequence comprises an A at X1, an N>C mutation at X2, and a U at X3, wherein N is A, G, or U (e.g., which is configured to bind to PTC 5′-UGA-3′).

In some embodiments, the mutation is selected from the group consisting of A>U, G>U, or C>U at position X2, relative to the endogenous tRNA. In some embodiments, the anticodon sequence comprises an A at X1, an N>U mutation at X2, and a C at X3, wherein N is A, G, or C (e.g., which is configured to bind to PTC 5′-UAG-3′). In some embodiments, the anticodon sequence comprises an A at X1, a N>U mutation at X2, and C at X3, wherein N is A, G, or C (e.g., which is configured to bind to FTC 5′-UAG-3′). In some embodiments, the anticodon sequence comprises an A at X1, a N>U mutation at X2, and a U at X3, wherein N is A, G, or C (e.g., which is configured to bind to FTC 5′-UAA-3′).

In some embodiments, the prime editor installs the mutation (e.g., transition or transversion) at position X3. In some embodiments, the mutation is selected from the group consisting of A>U, G>U, and C>U, relative to the endogenous tRNA. In some embodiments, the anticodon sequence comprises an A at X1, a C at X2, and a N>U at X3, wherein N is an A, G, or C (e.g., which is configured to bind to FTC 5′-UGA-3′). In some embodiments, the anticodon sequence comprises an A at X1, a U at X2 and a N>U at X3, wherein N is an A. G, or C (e.g., which is configured to bind to FTC 5′-UAA-3′).

In some embodiments, the mutation is selected from the group consisting of U>C, A>C, and G>C at position X3, relative to the endogenous tRNA. In some embodiments, the anticodon sequence comprises an A at X1, a U at X2 and a N>C at X3, wherein N is U, A, or G (e.g., which is configured to bind to PTC 5′-UAG-3′)

In some embodiments, the methods comprise a pegRNA comprising a spacer sequence, a gRNA core, and an extension arm. In some embodiments, the pegRNA further comprises a stabilizing 3′-tevopreQi motif. In some embodiments, the pegRNA directs the prime editor to install an edit at the target site located between positions +1 and +40, between positions +5 and +35, between positions +10 and +30, and between positions +15 and +25, relative to a first editable base located 3′ of a pegRNA-directed nick. In some embodiments, the pegRNA directs the prime editor to install an edit at a target site between positions +10 and +20 or between +11 and +17, relative to a first editable base located 3′ of a pegRNA-directed nick. Other installation sites are also possible in other embodiments.

In some embodiments, an extension arm of a pegRNA comprises a DNA synthesis template and a primer binding site (PBS). Without wishing to be bound by theory, the DNA synthesis template encodes via a polymerase (e.g., a reverse transcriptase) a single stranded DNA flap containing the genetic change of interest (e.g., nonsense suppressor anticodon sequence), which then integrates into the endogenous DNA sequence by replacing the corresponding endogenous strand, thereby installing the desired genetic change.

Accordingly, in some embodiments, the DNA synthesis template encodes an opal PTC (e.g., 5′-UGA-3′), an ochre PTC (e.g., 5′-UAA-3′), or an amber PTC (e.g., 5′-UAG-3′) (e.g., when the extension arm is at a 3′ end of the pegRNA). In some embodiments, the DNA synthesis template encodes a sequence that is complementary to an opal PTC (e.g., 5′-UCA-3′), an ochre PTC (e.g., 5′-UUA-3′), or an amber PTC (e.g., 5′-CUA-3′) (e.g., when the extension arm is at a 5′ end of the pegRNA). Likewise, in some embodiments, the DNA synthesis template encodes the C70U mutation to be installed at the target site of the DNA sequence encoding the acceptor stem domain of the endogenous tRNA. In some embodiments, the DNA synthesis template encodes the single base nucleotide to be installed at the target site of the DNA sequence encoding the variable arm domain of the endogenous tRNA.

Additionally, in some embodiments, the DNA synthesis template further encodes one or more PAM-disrupting mutation and/or MMR-evading mutations as described in U.S. Patent Application Ser. No. 63/136,194, filed Jan. 11, 2021, and International Patent Application No. PCT/US2022/012054, filed Jan. 11, 2022, both of which are herein incorporated by reference in their entirety.

In other embodiments, the methods disclosed herein may further require a gRNA and/or a second pegRNA.

In some embodiments, the pegRNA comprises any one of the spacer sequences and an extension arms listed in Table 2. In some embodiments, the endogenous tRNA is any tRNA listed in Table 1.

Aspects of the disclosure relate to compositions comprising a prime editor and a pegRNA that are capable of editing an endogenous tRNA into a suppressor tRNA. Any prime editor known in the art may be used to edit the endogenous tRNA into a suppressor tRNA. In some embodiments, the pegRNA comprises a spacer sequence, a core gRNA, and an extension arm comprising a DNA synthesis template and a primer binding site. In some embodiments, the pegRNA is tailored to maximize editing efficiency. In some embodiments, the DNA synthesis template encodes a nonsense suppressor anticodon to be installed at a target site of the DNA sequence encoding the anticodon sequence of the endogenous tRNA. In some embodiments, the nonsense suppressor anticodon is selected from the group consisting of 5′-UUA-3′, 5′-UCA-3′, and 5′-CUA-3′.

The DNA synthesis template may encode other edits in other embodiments. For example, in some embodiments, the DNA synthesis template encodes a C70U mutation to be installed at the target site of the DNA sequence encoding an acceptor stem domain of the endogenous tRNA. Alternatively, or additionally, the DNA synthesis template may encode for a single base nucleotide insertion into the DNA sequence encoding a variable arm domain of the endogenous tRNA molecule. The DNA synthesis template further encodes a PAM-disrupting mutation and/or an MMR-evading mutation, relative to the endogenous tRNA, in some embodiments.

In some embodiments, the compositions further comprise a sgRNA and/or a second pegRNA, for example, as may be needed for PE3 and twinPE prime editors, respectively.

Some aspects of the present disclosure relate to methods using twinPE prime editors to edit one or more domains of the endogenous tRNA. In some embodiments, the methods comprise editing both strands of a DNA sequence encoding the endogenous tRNA at a target site to be edited. Target sites include, but are not limited to, the D-arm domain, T-arm domain, acceptor stem domain, variable arm domain, and the anticodon arm domain comprising the anticodon sequence of the endogenous tRNA. In some embodiments, the methods comprise contacting the DNA sequence with a first prime editor complex and a second prime editor complex. Each of the first and second prime editor complexes comprise (1) a prime editor (e.g., PE2) comprising (i) a napDNAbp and a polymerase (e.g., a polypeptide having an RNA-dependent DNA polymerase activity) and (2) a pegRNA comprising a spacer sequence, gRNA core, an extension arm comprising a DNA synthesis template and a primer binding site.

In some embodiments, the DNA synthesis template of the pegRNA of the first prime editor complex encodes a first single stranded DNA sequence. In some embodiments, the DNA synthesis template of the pegRNA of the second prime editor complex encodes a second single-stranded DNA sequence. In some embodiments, the first single strand DNA sequence and the second single stranded DNA sequence encode a nonsense suppressor anticodon sequence to be installed at the target site of the DNA sequence encoding the anticodon sequence of the anticodon arm domain of the endogenous tRNA. In some embodiments, the first single strand DNA sequence and the second single stranded DNA sequence encode a premature termination sequence to be installed at the target site of the DNA sequence encoding the anticodon sequence of the anticodon arm domain of the endogenous tRNA. In some embodiments, the first single strand DNA sequence and the second single stranded DNA sequence encode a C70U mutation to be installed at the target site of the DNA sequence encoding the acceptor stem domain of the endogenous tRNA. In some embodiments, the first single strand DNA sequence and the second single stranded DNA sequence encode a single nucleotide insertion to be installed at the target site of the DNA sequence encoding the variable arm domain of the endogenous tRNA. In some embodiments, the first single strand DNA sequence and the second single stranded DNA sequence further encode PAM-disrupting mutations and/or MMR-evading mutations.

In some embodiments, the first single-stranded DNA sequence and the second single-stranded DNA sequence each comprises a region of complementarity to each other, such that they form a duplex comprising the edited portion, relative to the DNA sequence at the target site to be edited. In some embodiments, the duplex is integrated into the target site to be edited. In some embodiments, integrating the duplex into the target site installs the nonsense suppressor anticodon at the target site of the DNA sequence encoding the anticodon sequence of the endogenous tRNA. In some embodiments, the nonsense suppressor anticodon has the sequence 5′-UUA-3′ and is configured to bind to an ochre stop codon having sequence 5′-UAA-3′. In some embodiments, the nonsense suppressor anticodon has the sequence 5′-UCA-3′ configured to bind to an opal stop codon having sequence 5′-UGA-3′. In some embodiments, the nonsense suppressor anticodon has the sequence 5′-CUA-3′ configured to bind to an amber stop codon having sequence 5′-UAG-3′.

In some embodiments, integrating the duplex into the target site installs a C70U mutation in the DNA sequence encoding an acceptor stem domain of the endogenous tRNA. In some embodiments, installing the C70U mutation creates a G3:U70 base pair in the acceptor stem domain of the tRNA. In some embodiments, having the G3:U70 base pair in the acceptor stem domain of the tRNA causes the tRNA to be charged with the non-cognate amino acid alanine via the alanine-aminoacyl-tRNA synthetase. Other edits within the acceptor stem domain, leading to the incorporation of other non-cognate amino acids, is also possible in other embodiments.

In some embodiments, integrating the duplex into the target site installs a single base nucleotide insertion in the DNA sequence encoding a variable arm domain of the endogenous tRNA. In some cases, installing the additional nucleotide base, relative to the unedited endogenous tRNA, causes the tRNA to be charged with a noncognate amino acid serine via the serine-aminoacyl-tRNA synthetase.

In some embodiments, integrating the duplex into the target site further installs a PAM-disrupting mutation and/or an MMR-evading mutation in the DNA sequence.

In some embodiments, the pegRNAs comprise any protospacer sequence and pegRNA extension sequence listed in Table 2. In some embodiments, the endogenous tRNA is any tRNA listed in Table 1.

Other aspects of the disclosure relate to compositions comprising twinPEs for editing endogenous tRNAs into suppressor tRNAs. In some embodiments, the compositions comprise a first and second prime editor complex. In some embodiments, the first and second prime editor complexes comprises (1) a prime editor comprising (i) a nucleic acid programmable DNA binding protein (napDNAbp), and (ii) a polypeptide having an RNA-dependent DNA polymerase activity; and (2) a pegRNA comprising a spacer sequence, gRNA core, an extension arm comprising a DNA synthesis template and a primer binding site (PBS). In some embodiments, the DNA synthesis template of the pegRNA of the first prime editor complex encodes a first single-stranded DNA sequence and the DNA synthesis template of the pegRNA of the second prime editor complex encodes a second single-stranded DNA sequence. In some embodiments, the first single-stranded DNA sequence and the second single-stranded DNA sequence each comprises a region of complementarity to the other. In some embodiments, the first single-stranded DNA sequence and the second single-stranded DNA sequence form a duplex comprising an edited portion as compared to the DNA sequence at the target site to be edited, which integrates into the target site to be edited.

In some embodiments, the pegRNAs comprise any spacer sequence and pegRNA extension sequence listed in Table 2. In some embodiments, the endogenous tRNA is any tRNA listed in Table 1.

Aspects of the disclosure relate to pegRNAs for editing a DNA sequence encoding an endogenous tRNA by prime editing into a suppressor tRNA. In some embodiments, the pegRNA comprises a spacer sequence, a gRNA core, and an extension arm comprising a DNA synthesis template and a primer binding site. In some embodiments, the pegRNA further comprises a stabilizing 3′-tevopreQi motif. In some embodiments, the pegRNA is configured to bind to a DNA sequence encoding an endogenous tRNA.

In some embodiments, the DNA synthesis template encodes an opal PTC (e.g., 5′-UGA-3′), an ochre PTC (e.g., 5′-UAA-3′), or an amber PTC (e.g., 5′-UAG-3′) (e.g., when the extension arm is at a 3′ end of the pegRNA). In some embodiments, the DNA synthesis template encodes a sequence that is complementary to an opal PTC (e.g., 5′-UCA-3′), an ochre PTC (e.g., 5′-UUA-3′), or an amber PTC (e.g., 5′-CUA-3′) (e.g., when the extension arm is at a 5′ endo of the pegRNA). Likewise, in some embodiments, the DNA synthesis template encodes the C70U mutation to be installed at the target site of the DNA sequence encoding the acceptor stem domain of the endogenous tRNA. In some embodiments, the DNA synthesis template encodes the single base nucleotide to be installed at the target site of the DNA sequence encoding the variable arm domain of the endogenous tRNA.

Additionally, in some embodiments, the DNA synthesis template further encodes one or more PAM-disrupting mutation and/or MMR-evading mutations. In some embodiments, the pegRNA comprises any one of the spacer sequences and an extension arm listed in Table 2. In some embodiments, the endogenous tRNA is any tRNA listed in Table 1.

Other aspects of the disclosure relate to a complex comprising a prime editor (e.g., PE1, PE2, PE3, and/or twinPE) and a pegRNA for editing a DNA sequence encoding an endogenous tRNA by prime editing into a suppressor tRNA. In some embodiments, the pegRNA comprises a spacer sequence and an extension arm. In some embodiments, the pegRNAs comprise any protospacer sequence and pegRNA extension sequence listed in Table 2. In some embodiments, the endogenous tRNA is any tRNA listed in Table 1.

Aspects of the disclosure relate to polynucleotides comprising a first nucleic acid sequence encoding a prime editor and a second nucleic acid sequence encoding a pegRNA. In some embodiments, the pegRNAs comprise any protospacer sequence and pegRNA extension sequence listed in Table 2. In some embodiments, the pegRNA comprises a protospacer sequence with at least 90%, at least 95%, at least 98%, at least 99%, at least 99.5%, or at least 99.8% sequence identity to any protospacer sequence listed in Table 2. In other embodiments, the pegRNA comprises an extension arm with at least 90%, at least 95%, at least 98%, at least 99%, at least 99.5%, or at least 99.8% sequence identity to any extension arm listed in Table 2.

Additional aspects of the disclosure relate to methods for changing the amino acid that is charged onto a tRNA in a subject in need thereof. In some embodiments, the methods comprise administering to the subject: (i) a prime editor and (ii) a pegRNA, wherein the prime editor and gRNA form a prime editing complex. In some embodiments, the prime editing complex binds to a DNA sequence encoding an acceptor stem domain of the tRNA. In some embodiments, the prime editing complex installs a mutation in the acceptor stem domain. In some embodiments, mutation results in the replacement of a cognate amino acid with a non-cognate amino acid.

In some embodiments, the cognate amino acid is selected from the group consisting of alanine, arginine, asparagine, aspartic acid, cysteine, glutamine, glutamic acid, glycine, histidine, isoleucine, leucine, methionine, phenylalanine, proline, serine, threonine, tryptophan, tyrosine, valine, pyrrolysine, and selenocysteine.

In some embodiments, the non-cognate amino acid is selected from the group consisting of alanine, arginine, asparagine, aspartic acid, cysteine, glutamine, glutamic acid, glycine, histidine, isoleucine, leucine, methionine, phenylalanine, proline, serine, threonine, tryptophan, tyrosine, valine, pyrrolysine, and selenocysteine.

In some embodiments, the tRNA comprises an anticodon sequence that encodes for the cognate amino acid but is charged with the non-cognate amino acid. For example, in some embodiments, the cognate amino acid is lysine and the non-cognate amino acid is alanine. In other embodiments, the cognate amino acid is lysine and the non-cognate amino acid is serine.

In some embodiments, the act of editing an anticodon sequence of an endogenous tRNA to create a suppressor tRNA configured to bind to a premature termination codon may alter the aminoacylation of the endogenous tRNA, as described by Wang et al., “AAV-delivered suppressor tRNA overcomes a nonsense mutations in mice” Nature, 2022; 604 (7905): 348. For example, in some embodiments, an endogenous tRNA-Trp edited into a suppressor tRNA-Trp with an anticodon designed to bind to an amber stop codon (5′-UAG-3′) is charged with a lysine. In some embodiments, endogenous tRNA-Gln edited into a suppressor tRNA-Gln with an anticodon designed to bind to an amber stop codon (5′-UAG-3′) is charged with a lysine.

In some embodiments, the pegRNA comprises any one of the spacer sequences and an extension arm listed in Table 2. In some embodiments, the endogenous tRNA is any tRNA listed in Table 1.

Installing Suppressor tRNA Via Replacing Endogenous tRNAs

Aspects of the disclosure relate to methods using prime editing to replace endogenous tRNAs with suppressor tRNAs. As such, certain embodiments relate to overwriting an existing RNA with a suppressor tRNA. The RNA gene to be overwritten is, according to some embodiments, highly expressed, relative to the desired endogenous tRNA to be edited. The level of expression is easily determined using known techniques in the art, such as for example, high throughput gene expression profiling.

In some embodiments, the highly expressed RNA gene to be edited may be any suitable RNA gene known to the skilled artisan. In some embodiments, the highly expressed RNA gene is an endogenous tRNA gene.

In some embodiments, the endogenous tRNA to be edited has a plurality of isodecoders that may be edited. In some embodiments, the endogenous tRNA has greater than or equal to 2, greater than or equal to 3, greater than or equal to 4, greater than or equal to 5, greater than or equal to 6, greater than or equal to 7, greater than or equal to 8, greater than or equal to 9, greater than or equal to 10, greater than or equal to 15, greater than or equal to 20, greater than or equal to 25, greater than or equal to 30, greater than or equal to 35, greater than or equal 40, greater than or equal to 45, or greater than or equal to 50 isodecoders. In some embodiments, the endogenous tRNA has less than or equal to 50, less than or equal to 45, less than or equal to 40, less than or equal to 35, less than or equal to 30, less than or equal to 25, less than or equal to 20, less than or equal to 15, less than or equal to 10, less than or equal to 9, less than or equal to 8, less than or equal to 7, less than or equal to 6, less than or equal to 5, less than or equal to 4, less than or equal to 3, or less than or equal to 2 isodecoders. Any method known in the art by the skilled artisan may be used to replace an endogenous tRNA gene with a suppressor tRNA gene. In some embodiments, standard prime editing techniques are used to install the desired edits. In some embodiments, twin prime editing (twinPE), also known as “dual flap” prime editing, is used to install the edits. Without wishing to be bound by any particular theory, it is generally believed that twinPE may provide higher editing efficiencies due to the large size (e.g., length) of the desired edit. In some embodiments, the prime editor used to install the edits comprises PE2, PE3, PE4, PE5, PE2max, PE3max, PE4max, PE5max, twinPE, or Prime-del.

Installing Suppressor tRNA Via Gene Insertion

Aspects of the disclosure relate to methods for inserting a new suppressor tRNA gene into a target site of an organism's genome. Without wishing to be bound by theory, this approach requires insertion of a small gene rather than a local edit of a subset of endogenous tRNA bases, but may offer complementary advantages such as the lack of dependence on the presence, sequence, and dispensability of an endogenous tRNA gene in a specific target organism or patient. Any suitable method known in the art may be used to insert the new suppressor tRNA gene into the target site. Exemplary methods, include but are not limited to, prime editing methods (e.g., twinPE), prime editing methods coupled with integrase or recombinase enzymes, CRISPR-associated transposases (CASTs) and other targeted gene insertion technologies22-25 to achieve insertion of a suppressor tRNA or a suppressor tRNA expression cassette into the human genome is likewise also envisioned.

Accordingly, in some embodiments, the methods comprise inserting a suppressor tRNA gene into a target site in a genome (e.g., human genome) using prime editing (e.g., twinPE). In some embodiments, the methods comprise contacting the target site with (i) a prime editor and (ii) a pegRNA. In some embodiments, the prime editor comprises a fusion protein comprising a napDNAbp and a polymerase.

In some embodiments, the pegRNA comprises a spacer sequence, a gRNA core, and an extension arm comprising a DNA synthesis template and a primer binding site (PBS). In some embodiments, the spacer sequence comprises a region of complementarity to a target strand (e.g., protospacer sequence) of a double stranded target tRNA gene sequence in the subject. In some embodiments, the gRNA core associates with the napDNAbp. In some embodiments, the DNA synthesis template comprises a region of complementarity to the non-target strand of the double-stranded target tRNA gene sequence and encodes the suppressor tRNA gene sequence to be installed within the target site. In some embodiments, the primer binding site comprises a region of complementarity to a non-target strand of the double-stranded target tRNA gene sequence.

In some embodiments, the prime editor and the pegRNA install the suppressor tRNA gene sequence in the target site in the genome (e.g., human genome). In some embodiments, installation of the suppressor tRNA gene results in the indefinite expression of the suppressor tRNAs gene. Any suitable pegRNA, or pairs of pegRNAs, may be used to replace an endogenous tRNA with a suppressor tRNA, such the exemplary pegRNAs provided in Table 5.

In some embodiments, the suppressor tRNA gene encodes for a suppressor tRNA charged with an amino acid. In some embodiments, the amino acid is selected from the group consisting of alanine, arginine, asparagine, aspartic acid, cysteine, glutamine, glutamic acid, glycine, histidine, isoleucine, leucine, methionine, phenylalanine, proline, serine, threonine, tryptophan, tyrosine, valine, pyrrolysine, and selenocysteine.

In some embodiments, the suppressor tRNA gene further encodes for a suppressor tRNA with a nonsense suppressor anticodon that is complementary to a premature termination codon. For example, in some embodiments, the nonsense suppressor anticodon is 5′-UCA-3′ and binds to an opal premature termination codon having sequence 5′-UGA-3′. In some embodiments, the nonsense suppressor anticodon is 5′-UUA-3′ and binds to an ochre premature termination codon having sequence 5′-UAA-3′. In other embodiments, the nonsense suppressor anticodon is 5′-CUA-3′ and binds to an amber premature termination codon having sequence 5′-UAG-3′.

In some embodiments, the suppressor tRNA encodes for one or more mutations, relative to an endogenous tRNA. In some embodiments, the suppressor tRNA encodes for a tRNA and its cognate amino acid, but includes one or more mutations in one or more domains that results in the suppressor tRNA being charged with a non-cognate amino acid. Any mutation known in the art that results in amino acid misincorporation is envisioned herein. For example, in some embodiments, the suppressor tRNA gene encodes a Lys-tRNA-CUU comprising a C70U mutation in the acceptor stem domain. In this embodiment, expression of the suppressor tRNA gene would result in production of Ala-tRNA-CCU, instead of the endogenous Lys-tRNA-CUU. Other mutations are also possible in other embodiments. For example, in some embodiments, the suppressor tRNA gene encodes a single base nucleotide insertion in a variable arm domain, relative to an endogenous tRNA.

In some embodiments, the suppressor tRNA gene may be inserted into any suitable target site within the genome. In some embodiments, the target site is a safe harbor locus site. Without wishing to be bound by theory, genomic safe harbor locus sites are sites in the genome that are able to accommodate the integration of new genetic material in a manner that ensures that the newly inserted genetic elements function properly and do not cause alternations of the host genome posing a risk to the host cell or organism.

In some embodiments, any suitable safe harbor locus site (e.g., any currently known site or yet to be determined sites) may be used as the target site for gene insertion. In some embodiments, the safer harbor locus site comprises the ROSA26 gene, the AAVS1 gene, or the CCR5 gene.

In some embodiments, the target site is a general expression site. Any suitable general expression site (e.g., any currently known site or yet to be determined sites) may be used as the target site for gene insertion. For example, in some embodiments, the general expression site comprises the albumin gene (ALB gene).

Other aspects of the disclosure relate to inserting a suppressor tRNA gene into a genome using a prime editor fusion protein comprising a napDNAbp, a polymerase, and a recombinase and a pegRNA. Without wishing to bound by theory, recombinases, such as serine integrases (e.g., Bxb1) are art recognized enzymes capable of performing site-specific recombination. Site-specific recombination is an art recognized process in which DNA strand exchange takes place between 2 DNA segments (e.g., 2 different double strand DNAs) possessing at least a certain degree of sequence homology. The enzymes recognize and bind to short specific DNA recognition sites (e.g., a first recognition site located on the first double stranded DNA and a second recognition site located on a second double stranded DNA), at which they cleave the DNA backbone, exchange the two DNA helices involved, and rejoin the DNA strands. In some embodiments, the first and second recognition sites comprise identical sequences. In other embodiments, the first and second recognition sites comprise different sequences (e.g., attP and attB of phage integrase).

In some embodiments, the method comprises a circular DNA plasmid that encodes the suppressor tRNA gene to be inserted into the target site. In some embodiments, the suppressor tRNA gene comprises an anticodon sequence that is complementary to a premature termination sequence (e.g., complementary the following PTCs: 5′-UUA-3′, 5′-UCA-3′, 5′-CUA-3′). In some embodiments, the circular DNA plasmid encodes for the suppressor tRNA molecule comprising a C70U mutation in the acceptor stem domain of the tRNA. In some embodiments, the circular DNA plasmid encodes for the suppressor tRNA molecule comprising a single nucleotide insertion (e.g., mutation) in a variable arm domain of the suppressor tRNA, relative to an endogenous tRNA.

In some embodiments, the circular DNA plasmid comprises a first recombinase recognition site (e.g., AttP). In some embodiments, the first recombinase recognition site comprises an AttB sequence with a sequence identity of at least 85%, at least 90%, at least 95%, at least 98%, at least 99%, or at least 99.5% of SEQ ID NOs: 1-9 and 45711-45712. In some embodiments, the first recombinase recognition site comprises an AttP sequence with a sequence identity of at least 85%, at least 90%, at least 95%, at least 98%, at least 99%, or at least 99.5% of SEQ ID NOs: 10-17 and 45713-45715.

In some embodiments, a pegRNA comprises a spacer sequence, a gRNA core, and an extension arm comprising a DNA synthesis template and a primer binding site (PBS). Those of skill in the art will understand that the pegRNA guides the prime editor to the target site and encodes the edit to be installed into the human genome. In some embodiments, the DNA synthesis template encodes a single stranded DNA sequence encoding a second recombinase recognition site (e.g., a AttP or AttB). In some embodiments, the second recombinase recognition site comprises an AttB sequence with a sequence identity of at least 85%, at least 90%, at least 95%, at least 98%, at least 99%, or at least 99.5% of SEQ ID NOs: 1-9 and 45711-45712. In some embodiments, the second recombinase recognition site comprises an AttP sequence with a sequence identity of at least 85%, at least 90%, at least 95%, at least 98%, at least 99%, or at least 99.5% of SEQ ID NOs: 10-17 and 45713-45715.

In some embodiments, the method comprises placing the integrase in contact with the first recombination recognition site and second recombination recognition site. Upon being placed in contact, the integrase recombines the circular plasmid comprising the first recombination recognition site with the second recombination site that was previously inserted into the human genome at the target site (e.g., safe harbor locus) via prime editing. In certain embodiments, this permanently inserts the desired suppressor tRNA gene into the human genome at the target site (e.g., ROSA26, CCR5, and AAVS1). In some embodiments, installation of the suppressor tRNA gene at the target site (e.g., safe harbor locus site or general expression site) results in the indefinite expression of the suppressor tRNAs gene.

Other aspects relate to methods for treating a disease caused by premature termination codons, the method comprising mutating an endogenous tRNA gene into a suppressor tRNA gene using prime editing, the method comprising administering to a subject (i) a prime editor and (ii) a pegRNA, wherein the suppressor tRNA gene encodes a suppressor tRNA molecule comprising an anticodon sequence comprising ochre stop codon, an opal stop codon, or an amber stop codon.

Additional aspects of the disclosure relate to methods for treating a disease caused by premature termination codons, the method comprising installing a suppressor tRNA gene into a target site in a human genome using prime editing, the method comprising administering to a subject (i) a prime editor and (ii) a pegRNA, wherein the suppressor tRNA gene encodes a suppressor tRNA molecule comprising an anticodon sequence comprising ochre stop codon, an opal stop codon, or an amber stop codon,

Non-limiting examples of diseases caused by premature termination codons (e.g., nonsense mutations) include cystic fibrosis, beta thalassemia, Hurler syndrome, Dravet syndrome, Duchenne muscular dystrophy, Usher syndrome, and hemophilia.

Methods for Selecting Suppressor tRNA Genes

In some aspects, the current disclosure relates to one or more methods of selecting a suppressor tRNA gene. In some embodiments, the method comprises creating a reporter cell line comprising a reporter construct comprising a constitutively expressed fusion protein comprising a first biomarker protein, a premature termination codon (PTC) sequence, a ribosomal skipping element, and a second biomarker protein different than the first biomarker protein. In some embodiments, the methods comprise creating a gene library encoding the sequences of all human tRNA sequences. In some cases, the tRNA sequences comprise the same three-base pair anticodon that is complimentary to the PTC in the reporter construct. In some embodiments, the methods comprise introducing the library into the reporter cell line. In some embodiments, the methods comprise sorting cells that express the second biomarker protein and determining which tRNA sequences are enriched in the sorted population.

In some embodiments, the PTC is TGA. In other embodiments, the PTC is TAG. In other cases, the PTC is TAA, according to some embodiments. In some cases, the biomarker (e.g., first or second biomarker) is a fluorescent protein. In some instances, the first biomarker is a mCherry fluorescent protein. In some embodiments, the second biomarker is a green fluorescent protein (GFP).

In some embodiments, each cell contains a single copy of the reporter construct. Techniques to ensure that each cell only contains a single copy of the reporter construct are known in the art, and the skill artisan may use any of said techniques in any of the methods disclosed herein. In some embodiments, the PTC in the reporter construct can be replaced with any amino acid variant without altering expression levels of the second biomarker protein.

In some embodiments, the gene library is cloned into a lentiviral backbone, although other backbones may be used. Accordingly, in some embodiments, any suitable lentiviral backbone known in the art may be used as the lentiviral backbone in any of the methods disclosed herein.

In some embodiments, the lentiviral backbone comprises an exogenous promoter. Any suitable exogenous promoter known in the art by the skilled artisan may be used in any of the methods disclosed herein. In some embodiments, the promoter comprises a human U6. In other embodiments, the promoter comprises a minimal U6 promoters. Alternatively, in some embodiments, the lentiviral backbone does not comprise an exogenous promoter. For example, in some cases, the encoded tRNA sequence comprises an endogenous tRNA protomer capable of driving expression of the tRNA gene sequence.

In some embodiments, the genes within the gene library further comprise a leader sequence. Any suitable leader sequence known to the skilled artisan may be used in any of the methods disclosed herein, such as those disclosed in Table 3. In some embodiments, the leader sequence is positioned to precede the mature tRNA sequence. However, other arrangements are also possible in other embodiments.

In some embodiments, the genes within the gene library further comprise a termination sequence. Any suitable termination sequence known to the skilled artisan may be used in any of the methods as described herein, such as those disclosed in Table 4. For instance, in some embodiments, the termination sequence comprises a poly T tail. In some embodiments, the termination sequence comprises four, five, or seven thymidine tracks. In some embodiments, the termination sequence is within 100 bp of the mature tRNA sequence.

In some embodiments, the selected suppressor tRNA gene may encode for any known tRNA gene of any species known to the skilled artisan. For example, in some embodiments, the encoded tRNA gene is a human tRNA gene. In some embodiments, the selected suppressor tRNA gene encodes for Leu-TAA-1-1, Leu-TAA-2-1, Leu-TAA-3-1, or Leu-TAA-4-1.

In some embodiments, the methods relate to a method of selecting a pegRNA to edit an endogenous tRNA gene. In some embodiments, the methods comprise creating a reporter cell line comprising a reporter construct comprising a constitutively expressed fusion protein comprising a first biomarker protein, a premature termination codon (PTC) sequence, a ribosomal skipping element, and a second biomarker protein different than the first fluorescent protein. In some embodiments, the methods further comprise creating a gene library encoding pegRNAs that target every tRNA sequence in the genome and which convert the natural tRNA anticodon of said tRNA sequence to a PTC. In some embodiments, the methods further comprise introducing the gene library and a prime editor into the cell line and sorting cells that express the second biomarker protein and determining which pegRNA sequences are enriched in the sorted population.

### Prime Editors

The present disclosure contemplates using prime editors comprising fusion proteins, wherein the fusion proteins comprise a nucleic acid programmable DNA binding protein (napDNAbp) domain and a polymerase (e.g., reverse transcriptase) domain. Any suitable napDNAbp and polymerase known in the art may be combined into a single fusion protein with any suitable structural configuration, in accordance with some embodiments.

For example, the fusion protein may comprise, from the N-terminus to the C-terminus direction, a napDNAbp fused to a polymerase. In other embodiments, the fusion protein may comprise from the N-terminus to the C-terminus direction, a polymerase fused to a napDNAbp. The fused domain may optionally be joined by a linker, e.g., an amino acid sequence. In other embodiments, the fusion proteins may comprise the structure NH2-[napDNAbp]-[polymerase]-COOH; or NH2-[polymerase]-[napDNAbp]-COOH, wherein each instance of “]-[” indicates the presence of an optional linker sequence. In embodiments wherein the polymerase is a reverse transcriptase, the fusion proteins may comprise the structure NH2-[napDNAbp]-[RT]-COOH; or NH2-[RT]-[napDNAbp]-COOH, wherein each instance of “]-[” indicates the presence of an optional linker sequence.

Since prime editors, and hence napDNAbps and polymerases, are well-known in the art, and the amino acid sequences are readily available, this disclosure is not meant in any way to be limited to those specific napDNAbps and/or polymerases identified herein. Non-limiting examples of prime editors contemplated herein may be found in U.S. Provisional Application No. 62/820,813, U.S. Provisional Application No. 62/858,958, U.S. Provisional Application No. 62/889,996, U.S. Provisional Application No. 62/922,654, U.S. Provisional Application No. 62/913,553, U.S. Provisional Application No. 62/973,558, U.S. Provisional Application No. 62/931,195, U.S. Provisional Application No. 62/944,231, U.S. Provisional Application No. 62/974,537, U.S. Provisional Application No. 62/991,069, U.S. Provisional Application No. 63/100,548, U.S. Provisional Application No. 63/022,397, U.S. Provisional Application No. 63/116,785, International PCT Application No. PCT/US2020/023721, International PCT Application No. PCT/US2020/023553, International PCT Application No. PCT/US2020/023583, International PCT Application No. PCT/US2020/023730, International PCT Application No. PCT/US2020/023713, International PCT Application No. PCT/US2020/023712, International PCT Application No. PCT/US2020/023727, International PCT Application No. PCT/US2020/023724, International PCT Application No. PCT/US2020/023725, International PCT Application No. PCT/US2020/023728, International PCT Application No. PCT/US2020/023732, International PCT Application No. PCT/US20201023723, International PCT Application No. PCT/US2021/031439, and U.S. Pat. No. 11,440,770, issued on Sep. 20, 2022, all of which are herein incorporated by reference in their entirety.

In some embodiments, the napDNAbp domain and the polymerase domain are fused together without a linker. In other embodiments, the napDNAbp domain is fused to the polymerase domain via a linker. Any suitable linker known in the art may be used to fuse the napDNAbp domain and the polymerase domain. For example, in some embodiments, the linker is a peptide, a polypeptide, a protein, a nucleic acid, a polymer, a polysaccharide, or any combination thereof.

In some embodiments, the fusion proteins may comprise any suitable structural configuration. For example, the fusion protein may comprise from the N-terminus to the C-terminus direction, a napDNAbp fused to a polymerase (e.g., DNA-dependent DNA polymerase or RNA-dependent DNA polymerase, such as, reverse transcriptase). In other embodiments, the fusion protein may comprise from the N-terminus to the C-terminus direction, a polymerase (e.g., a reverse transcriptase) fused to a napDNAbp. The fused domain may optionally be joined by a linker, e.g., an amino acid sequence. In other embodiments, the fusion proteins may comprise the structure NH2-[napDNAbp]-[polymerase]-COOH; or NH2-[polymerase]-[napDNAbp]-COOH, wherein each instance of “]-[” indicates the presence of an optional linker sequence. In embodiments wherein the polymerase is a reverse transcriptase, the fusion proteins may comprise the structure NH2-[napDNAbp]-[RT]-COOH; or NH2-[RT]-[napDNAbp]-COOH, wherein each instance of “]-[” indicates the presence of an optional linker sequence.

In various embodiments, the prime editor fusion protein may have the following structure (referred to herein as “PE1”), which includes a Cas9 variant comprising an H840A mutation (i.e., a Cas9 nickase) and an M-MLV RT wild type, as well as an N-terminal NLS sequence (19 amino acids) and an amino acid linker (32 amino acids) that joins the C-terminus of the Cas9 nickase domain to the N-terminus of the RT domain. The PE1 fusion protein has the following structure: [NLS]-[Cas9(H840A)]-[linker]-[MMLV_RT(wt)].

In some embodiments, the prime editor fusion protein (referred to herein as “PE2”) comprises a Cas9(H840A) and a variant MMLV RT having the following structure: [NLS]-[Cas9(H840A)]-[linker]-[MMLV_RT(D200N)(T330P)(L603W)(T306K)(W313F)] and a desired pegRNA.

In one set of embodiments, a prime editor of the present disclosure may be a PE1, PE2, PE3, PE4, PE5, or TwinPE editor as described in Anzalone et al., “Search-and-replace genome editing without double-strand breaks or donor DNA,” Nature. 2019 December: 576(7785): 149-157; Anzalone et al., “Programmable deletion, replacement, integration, and inversion of large DNA sequences with twin prime editing,” Nat. Biotechnol. 2022 May; 40(5):731-740; and by Choi et al., “Precise genomic deletions using paired prime editing,” Nat. Biotechnol. 2022 February; 40(2):218-226, all of which are herein incorporated by reference in their entirety. In some embodiments, TwinPE comprises a pair of PE2 editors and two pegRNAs that target opposite strands of a double stranded nucleic acid (e.g., DNA).

In some embodiments, a PE3 prime editor comprises PE2 machinery and an additional sgRNA.

In some embodiments, a TwinPE editor comprises a first prime editor complex and a second prime editor complex. In some embodiments, the first prime editor complex comprises a first prime editor comprising a first nucleic acid programmable DNA binding protein (first napDNAbp) and a first polypeptide comprising an RNA-dependent DNA polymerase activity. The first prime editor complex further comprises a first prime editing guide RNA (first pegRNA) that binds to a first binding site on a first strand of the double-stranded DNA sequence upstream of the target site to be edited. In some embodiments, the first prime editor complex is a first PE2 editor. In some embodiments, the second prime editor complex comprises a second prime editor comprising a second nucleic acid programmable DNA binding protein (second napDNAbp) and a second polypeptide comprising an RNA-dependent DNA polymerase activity. The second prime editor complex further comprises a second prime editing guide RNA (second pegRNA) that binds to a second binding site on a second strand of the double-stranded DNA sequence upstream of the target site to be edited. In some embodiments, the second prime editor complex is a second PE2 editor. In some embodiments, the first pegRNA comprises a first DNA synthesis template encoding a first single-stranded DNA sequence and the second pegRNA comprises a second DNA synthesis template encoding a second single-stranded DNA sequence. In some embodiments, the first and the second single-stranded DNA sequence each comprise a region of complementarity to the other. In some embodiments, the first single-stranded DNA sequence and the second single-stranded DNA sequence form a duplex comprising an edited portion as compared to the DNA sequence at the target site to be edited.

napDNAbbp Domain

In some embodiments, a prime editor comprises a (napDNAbp) domain. Any suitable napDNAbp domain known in the art may be used in the prime editors described herein, such as those described in detail in U.S. Patent Application 63/136,194, titled “Prime editor variants, constructs, and methods of using the same” by David Liu, et al., filed on Jan. 11, 2021, which is incorporated herein by reference in its entirety. For example, in various embodiments, the napDNAbp may be any Class 2 CRISPR-Cas system, including any type II, type V. or type VI CRISPR-Cas enzyme. Given the rapid development of CRISPR-Cas as a tool for genome editing, there have been constant developments in the nomenclature used to describe and/or identify CRISPR-Cas enzymes, such as Cas9 and Cas9 orthologs. This application references CRISPR-Cas enzymes with nomenclature that may be old and/or new as described in U.S. Patent Application 63/136,194 (described elsewhere herein) or Makarova et al., The CRISPR Journal, Vol. 1, No. 5, 2018, which is incorporated herein by reference in its entirety.

Other napDNAbps are also possible in other embodiments. For example, in some embodiments, the napDNAbp comprises the canonical SpCas9, or any ortholog Cas9 protein, or any variant Cas9 protein—including any naturally occurring variant, mutant, or otherwise engineered version of Cas9—that is known or that may be made or evolved through a directed evolutionary or otherwise mutagenic process. In various embodiments, the Cas9 or Cas9 variants have a nickase activity, i.e., only cleave one strand of the target DNA sequence. In other embodiments, the Cas9 or Cas9 variants have inactive nucleases, i.e., are “dead” Cas9 proteins. Other variant Cas9 proteins that may be used are those having a smaller molecular weight than the canonical SpCas9 (e.g., for easier delivery) or having modified or rearranged primary amino acid structure (e.g., the circular permutant formats).

In various embodiments described herein, the prime editors comprise a napDNAbp, such as a Cas9 protein. These proteins are “programmable” by way of their becoming complexed with a guide RNA (or a pegRNA, as the case may be), which guides the Cas9 protein to a target site on the DNA which possess a sequence that is complementary to the spacer portion of the gRNA (or pegRNA) and also which possesses the required PAM sequence. However, in certain embodiment envisioned here, the napDNAbp may be substituted with a different type of programmable protein, such as a zinc finger nuclease or a transcription activator-like effector nuclease (TALEN). See U.S. patent application U.S. Ser. No. 12/965,590; U.S. Ser. No. 13/426,991 (U.S. Pat. No. 8,450,471); U.S. Ser. No. 13/427,040 (U.S. Pat. No. 8,440,431); U.S. Ser. No. 13/427,137 (U.S. Pat. No. 8,440,432); and U.S. Ser. No. 13/738,381, all of which are incorporated by reference herein in their entirety. In addition, TALENS are described in WO 2015/027134, U.S. Pat. No. 9,181,535, Boch et al., “Breaking the Code of DNA Binding Specificity of TAL-Type III EtTectors”, Science, vol. 326, pp. 1509-1512 (2009), Bogdanove et al., TAL Effectors: Customizable Proteins for DNA Targeting, Science, vol. 333, pp. 1843-1846 (2011), Cade et al., “Highly efficient generation of heritable zebrafish gene mutations using homo- and heterodimeric TALENs”, Nucleic Acids Research, vol. 40, pp. 8001-8010 (2012), and Cermak et al., “Efficient design and assembly of custom TALEN and other TAL effector-based constructs for DNA targeting”, Nucleic Acids Research, vol. 39, No. 17, e82 (2011), each of which are incorporated herein by reference. See also, for example, in Carroll et al., “Genome Engineering with Zinc-Finger Nucleases,” Genetics, August 2011, Vol. 188: 773-782; Durai et al., “Zinc finger nucleases: custom-designed molecular scissors for genome engineering of plant and mammalian cells,” Nucleic Acids Res, 2005, Vol. 33: 5978-90; and Gaj et al., “ZFN, TALEN, and CRISPR/Cas-based methods for genome engineering,” Trends Biotechnol. 2013, Vol. 31: 397-405, each of which are incorporated herein by reference in their entireties.

Any suitable napDNAbp may be used in the prime editors described herein. In various embodiments, the napDNAbp may be any Class 2 CRISPR-Cas system, including any type II, type V, or type VI CRISPR-Cas enzyme. Given the rapid development of CRISPR-Cas as a tool for genome editing, there have been constant developments in the nomenclature used to describe and/or identify CRISPR-Cas enzymes, such as Cas9 and Cas9 orthologs. This application references CRISPR-Cas enzymes with nomenclature that may be old and/or new. The skilled person will be able to identify the specific CRISPR-Cas enzyme being referenced in this Application based on the nomenclature that is used, whether it is old (i.e., “legacy”) or new nomenclature. CRISPR-Cas nomenclature is extensively discussed in Makarova et al., “Classification and Nomenclature of CRISPR-Cas Systems: Where from Here?,” The CRISPR Journal, Vol. 1. No. 5, 2018, the entire contents of which are incorporated herein by reference. The particular CRISPR-Cas nomenclature used in any given instance in this Application is not limiting in any way and the skilled person will be able to identify which CRISPR-Cas enzyme is being referenced.

For example, the following type II, type V, and type VI Class 2 CRISPR-Cas enzymes have the following art-recognized old (i.e., legacy) and new names. Each of these enzymes, and/or variants thereof, may be used with the prime editors described herein:

| | |
|---|---|
| Legacy nomenclature | Current nomenclature* |

type II CRISPR-Cas enzymes

| | |
|---|---|
| Cas9 | same |

type V CRISPR-Cas enzymes

| | |
|---|---|
| Cpf1 | Cas12a |
| CasX | Cas12e |
| C2c1 | Cas12b1 |
| Cas12b2 | same |
| C2c3 | Cas12c |
| CasY | Cas12d |
| C2c4 | same |
| C2c8 | same |
| C2c5 | same |
| C2c10 | same |
| C2c9 | same |

type VI CRISPR-Cas enzymes

| | |
|---|---|
| C2c2 | Cas13a |
| Cas13d | same |
| C2c7 | Cas13c |
| C2c6 | Cas13b |

*See Makarova et al., The CRISPR Journal, Vol. 1, No. 5, 2018

The below description of various napDNAbps which can be used in connection with the presently disclose prime editors is not meant to be limiting in any way. The prime editors may comprise the canonical SpCas9, or any ortholog Cas9 protein, or any variant Cas9 protein—including any naturally occurring variant, mutant, or otherwise engineered version of Cas9—that is known, or which can be made or evolved through a directed evolutionary or otherwise mutagenic process. In various embodiments, the Cas9 or Cas9 variants have a nickase activity, i.e., only cleave of strand of the target DNA sequence. In other embodiments, the Cas9 or Cas9 variants have inactive nucleases, i.e., are “dead” Cas9 proteins. Other variant Cas9 proteins that may be used are those having a smaller molecular weight than the canonical SpCas9 (e.g., for easier delivery) or having modified or rearranged primary amino acid structure (e.g., the circular permutant formats).

The prime editors described herein may also comprise Cas9 equivalents, including Cas12a (Cpf1) and Cas12b1 proteins which are the result of convergent evolution. The napDNAbps used herein (e.g., SpCas9, Cas9 variant, or Cas9 equivalents) may also contain various modifications that alter/enhance their PAM specificities. Lastly, the application contemplates any Cas9. Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a references SpCas9 canonical sequence or a reference Cas9 equivalent (e.g., Cas12a (Cpf1)).

The napDNAbp can be a CRISPR (clustered regularly interspaced short palindromic repeat)-associated nuclease. As outlined above, CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (mc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M. et al., Science 337:816-821(2012), the entire contents of which is hereby incorporated by reference.

In some embodiments, the napDNAbp directs cleavage of one or both strands at the location of a target sequence, such as within the target sequence and/or within the complement of the target sequence. In some embodiments, the napDNAbp directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a napDNAbp that is mutated to with respect to a corresponding wild-type enzyme such that the mutated napDNAbp lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A in reference to the canonical SpCas9 sequence, or to equivalent amino acid positions in other Cas9 variants or Cas9 equivalents.

As used herein, the term “Cas protein” refers to a full-length Cas protein obtained from nature, a recombinant Cas protein having a sequences that differs from a naturally occurring Cas protein, or any fragment of a Cas protein that nevertheless retains all or a significant amount of the requisite basic functions needed for the disclosed methods, i.e., (i) possession of nucleic-acid programmable binding of the Cas protein to a target DNA, and (ii) ability to nick the target DNA sequence on one strand. The Cas proteins contemplated herein embrace CRISPR Cas 9 proteins, as well as Cas9 equivalents, variants (e.g., Cas9 nickase (nCas9) or nuclease inactive Cas9 (dCas9)) homologs, orthologs, or paralogs, whether naturally occurring or non-naturally occurring (e.g., engineered or recombinant), and may include a Cas9 equivalent from any Class 2 CRISPR system (e.g., type II, V, VI), including Cas12a (Cpf1), Cas12e (CasX), Cas12b1 (C2c1), Cas12b2, Cas12c (C2c3), C2c4, C2c8, C2c5, C2c10, C2c9 Cas13a (C2c2), Cas13d, Cas13c (C2c7), Cas13b (C2c6), and Cas13b. Further Cas-equivalents are described in Makarova et al., “C2c2 is a single-component programmable RNA-guided RNA-targeting CRISPR effector,” Science 2016; 353(6299) and Makarova et al., “Classification and Nomenclature of CRISPR-Cas Systems: Where from Here?,” The CRISPR Journal, Vol. 1. No. 5, 2018, the contents of which are incorporated herein by reference.

The terms “Cas9” or “Cas9 nuclease” or “Cas9 moiety” or “Cas9 domain” embrace any naturally occurring Cas9 from any organism, any naturally-occurring Cas9 equivalent or functional fragment thereof, any Cas9 homolog, ortholog, or paralog from any organism, and any mutant or variant of a Cas9, naturally-occurring or engineered. The term Cas9 is not meant to be particularly limiting and may be referred to as a “Cas9 or equivalent.” Exemplary Cas9 proteins are further described herein and/or are described in the art and are incorporated herein by reference. The present disclosure is unlimited with regard to the particular Cas9 that is employed in the prime editor (PE) of the invention.

As noted herein, Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference).

Examples of Cas9 and Cas9 equivalents are provided as follows; however, these specific examples are not meant to be limiting. The primer editor of the present disclosure may use any suitable napDNAbp, including any suitable Cas9 or Cas9 equivalent.

### Wild Type Canonical SpCas9

In one embodiment, the primer editor constructs described herein may comprise the “canonical SpCas9” nuclease from S. pyogenes, which has been widely used as a tool for genome engineering and is categorized as the type II subgroup of enzymes of the Class 2 CRISPR-Cas systems. This Cas9 protein is a large, multi-domain protein containing two distinct nuclease domains. Point mutations can be introduced into Cas9 to abolish one or both nuclease activities, resulting in a nickase Cas9 (nCas9) or dead Cas9 (dCas9), respectively, that still retains its ability to bind DNA in a sgRNA-programmed manner. In principle, when fused to another protein or domain, Cas9 or variant thereof (e.g., nCas9) can target that protein to virtually any DNA sequence simply by co-expression with an appropriate sgRNA.

The prime editors described herein may include canonical SpCas9, or any variant thereof having at least 80%, at least 85%, at least 90%, at least 95%, or at least 99% sequence identity with a wild type Cas9 sequence provided above.

Other wild type SpCas9 sequences that may be used in the present disclosure, include: SpCas9 (Streptococcus pyogenes MGAS1882 wild type, NC_017053.1), SpCas9 (Streptococcus pyogenes MGAS1882 wild type, NC_017053.1), SpCas9 (Streptococcus pyogenes wild type, SWBC2D7W014), SpCas9 Streptococcus pyogenes wild type (Encoded product of SWBC2D7W014), SpCas9 (Streptococcus pyogenes MIGAS wild type, NC_002737.2), SpCas9 (Streptococcus pyogenes MIGAS wild type, Encoded product of NC_002737.2 (100% identical to the canonical Q99ZW2 wild type),

The prime editors described herein may include any of the above SpCas9 sequences, or any variant thereof having at least 80%, at least 85%, at least 90%, at least 95%, or at least 99% sequence identity thereto.

### Wild Type Cas9 Orthologs

In other embodiments, the Cas9 protein can be a wild type Cas9 ortholog from another bacterial species different from the canonical Cas9 from S. pyogenes. For example, the following Cas9 orthologs can be used in connection with the prime editor constructs described in this specification. In addition, any variant Cas9 orthologs having at least 80%, at least 85%, at least 90%, at least 95%, or at least 99% sequence identity to any of the below orthologs may also be used with the present prime editors.

LfCas9 (Lactobacillus fermentum wild type, GenBank: SNX31424.1 1), SaCas9 (Staphylococcus aureus wild type, GenBank: AYD60528.1), SaCas9 (Staphylococcus aureus), StCas9 (Streptococcus thermophilus, UniProtKB/Swiss-Prot: G3ECR1.2 Wild type), LcCas9 (Lactobacillus crispatus, NCBI Reference Sequence: WP_133478044.1, Wild type), PdCas9 (Pedicoccus damnosus, NCBI Reference Sequence: WP_062913273.1, Wild type), FnCas9 (Fusobaterium nucleatum, NCBI Reference Sequence: WP_060798984.1). EcCas9 (Enterococcus cecorum, NCBI Reference Sequence: WP_047338501.1, Wild type), AhCas9 (Anaerostipes hadrus, NCBI Reference Sequence: WP_044924278.1, Wild type), KvCas9 (Kandleria vitulina, NCBI Reference Sequence: WP_031589969.1, Wild type), EfCas9 (Enterococcus faecalis, NCBI Reference Sequence: WP_016631044.1, Wild type), Staphylococcus aureus Cas9, Geobacillus thermodenitrificans Cas9, ScCas9 (S. canis, 1375 AA, 159.2 kDa).

The prime editors described herein may include any of the above Cas9 ortholog sequences, or any variants thereof having at least 80%, at least 85%, at least 90%, at least 95%, or at least 99% sequence identity thereto.

The napDNAbp may include any suitable homologs and/or orthologs or naturally occurring enzymes, such as, Cas9. Cas9 homologs and/or orthologs have been described in various species, including, but not limited to, S. pyogenes and S. thermophilus. Preferably, the Cas moiety is configured (e.g., mutagenized, recombinantly engineered, or otherwise obtained from nature) as a nickase and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737: the entire contents of which are incorporated herein by reference. In some embodiments, a Cas9 nuclease has an inactive (e.g., an inactivated) DNA cleavage domain, that is, the Cas9 is a nickase. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the Cas9 orthologs in the above tables.

### Dead Cas9 Variant

In certain embodiments, the prime editors described herein may include a dead Cas9, e.g., dead SpCas9, which has no nuclease activity due to one or more mutations that inactive both nuclease domains of Cas9, namely the RuvC domain (which cleaves the non-protospacer DNA strand) and HNH domain (which cleaves the protospacer DNA strand). The nuclease inactivation may be due to one or mutations that result in one or more substitutions and/or deletions in the amino acid sequence of the encoded protein, or any variants thereof having at least 80%, at least 85%, at least 90%, at least 95%, or at least 99% sequence identity thereto.

As used herein, the term “dCas9” refers to a nuclease-inactive Cas9 or nuclease-dead Cas9, or a functional fragment thereof, and embraces any naturally occurring dCas9 from any organism, any naturally-occurring dCas9 equivalent or functional fragment thereof, any dCas9 homolog, ortholog, or paralog from any organism, and any mutant or variant of a dCas9, naturally-occurring or engineered. The term dCas9 is not meant to be particularly limiting and may be referred to as a “dCas9 or equivalent.” Exemplary dCas9 proteins and method for making dCas9 proteins are further described herein and/or are described in the art and are incorporated herein by reference.

In other embodiments, dCas9 corresponds to, or comprises in part or in whole, a Cas9 amino acid sequence having one or more mutations that inactivate the Cas9 nuclease activity. In other embodiments, Cas9 variants having mutations other than D10A and H840A are provided which may result in the full or partial inactivate of the endogenous Cas9 nuclease activity (e.g., nCas9 or dCas9, respectively). Such mutations, by way of example, include other amino acid substitutions at D10 and H820, or other substitutions within the nuclease domains of Cas9 (e.g., substitutions in the HNH nuclease subdomain and/or the RuvC1 subdomain) with reference to a wild type sequence such as Cas9 from Streptococcus pyogenes (NCBI Reference Sequence: NC_017053.1). In some embodiments, variants or homologues of Cas9 (e.g., variants of Cas9 from Streptococcus pyogenes (NCBI Reference Sequence: NC_017053.1) are provided which are at least about 70% identical, at least about 80% identical, at least about 90% identical, at least about 95% identical, at least about 98% identical, at least about 99% identical, at least about 99.5% identical, or at least about 99.9% identical to NCBI Reference Sequence: NC_017053.1. In some embodiments, variants of dCas9 (e.g., variants of NCBI Reference Sequence: NC_017053.1) are provided having amino acid sequences which are shorter, or longer than NC_017053.1 by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

In one embodiment, the dead Cas9 may be based on the canonical SpCas9 sequence of Q99ZW2 and comprise D10A and an H810A substitutions.

### Cas9 Nickase Variant

In one embodiment, the prime editors described herein comprise a Cas9 nickase. The term “Cas9 nickase” of “nCas9” refers to a variant of Cas9 which is capable of introducing a single-strand break in a double strand DNA molecule target. In some embodiments, the Cas9 nickase comprises only a single functioning nuclease domain. The wild type Cas9 (e.g., the canonical SpCas9) comprises two separate nuclease domains, namely, the RuvC domain (which cleaves the non-protospacer DNA strand) and HNH domain (which cleaves the protospacer DNA strand). In one embodiment, the Cas9 nickase comprises a mutation in the RuvC domain which inactivates the RuvC nuclease activity. For example, mutations in aspartate (D) 10, histidine (H) 983, aspartate (D) 986, or glutamate (E) 762, have been reported as loss-of-function mutations of the RuvC nuclease domain and the creation of a functional Cas9 nickase (e.g., Nishimasu et al., “Crystal structure of Cas9 in complex with guide RNA and target DNA,” Cell 156(5), 935-949, which is incorporated herein by reference). Thus, nickase mutations in the RuvC domain could include D10X, H983X, D986X, or E762X, wherein X is any amino acid other than the wild type amino acid. In certain embodiments, the nickase could be D10A, of H983A, or D986A, or E762A, or a combination thereof.

In various embodiments, the Cas9 nickase can having a mutation in the RuvC nuclease domain. Exemplary embodiments include: Cas9 nickase, Cas9 nickase (Streptococcus pyogenes Q99ZW2 Cas9 with D10X, wherein X is any alternate amino acid), Cas9 nickase (Streptococcus pyogenes Q99ZW2 Cas9 with E762X, wherein X is any alternate amino acid), Cas9 nickase (Streptococcus pyogenes Q99ZW2 Cas9 with H983X, wherein X is any alternate amino acid), Cas9 nickase (Streptococcus pyogenes Q99ZW2 Cas9 with D986X, wherein X is any alternate amino acid), Cas9 nickase (Streptococcus pyogenes Q99ZW2 Cas9 with D10A), Cas9 nickase (Streptococcus pyogenes Q99ZW2 Cas9 with E762A), Cas9 nickase (Streptococcus pyogenes Q99ZW2 Cas9 with H983A). Cas9 nickase (Streptococcus pyogenes Q99ZW2 Cas9 with D986A).

In another embodiment, the Cas9 nickase comprises a mutation in the HNH domain which inactivates the HNH nuclease activity. For example, mutations in histidine (H) 840 or asparagine (R) 863 have been reported as loss-of-function mutations of the HNH nuclease domain and the creation of a functional Cas9 nickase (e.g., Nishimasu et al., “Crystal structure of Cas9 in complex with guide RNA and target DNA,” Cell 156(5), 935-949, which is incorporated herein by reference). Thus, nickase mutations in the HNH domain could include H840X and R863X, wherein X is any amino acid other than the wild type amino acid. In certain embodiments, the nickase could be H840A or R863A or a combination thereof.

In various embodiments, the Cas9 nickase can have a mutation in the HNH nuclease domain. Exemplary embodiments, include but are not limited to, Cas9 nickase (Streptococcus pyogenes Q99ZW2 Cas9 with H840X, wherein X is any alternate amino acid), Cas9 nickase (Streptococcus pyogenes Q99ZW2 Cas9 with H840A), Cas9 nickase (Streptococcus pyogenes Q99ZW2 Cas9 with R863X, wherein X is any alternate amino acid), Cas9 nickase (Streptococcus pyogenes Q99ZW2 Cas9 with R863A). Any amino acid sequence or variant thereof having an amino acid sequence that has at least 80%, at least 85%, at least 90%, at least 95%, or at least 99% sequence identity to any of the exemplary embodiments disclosed herein are also contemplated.

In some embodiments, the N-terminal methionine is removed from a Cas9 nickase, or from any Cas9 variant, ortholog, or equivalent disclosed or contemplated herein. For example, methionine-minus Cas9 nickases include Cas9 nickase ((Met minus) Streptococcus pyogenes Q99ZW2 Cas9 with H840X, wherein X is any alternate amino acid), Cas9 nickase ((Met minus) Streptococcus pyogenes Q99ZW2 Cas9 with H840A), Cas9 nickase ((Met minus) Streptococcus pyogenes Q99ZW2 Cas9 with R863X, wherein X is any alternate amino acid), Cas9 nickase ((Met minus) Streptococcus pyogenes Q99ZW2 Cas9 with R863A). Any amino acid sequence or variant thereof having an amino acid sequence that has at least 80%, at least 85%, at least 90%, at least 95%, or at least 99% sequence identity to any of the exemplary embodiments disclosed herein are also contemplated.

### Other Cas9 Variants

Besides dead Cas9 and Cas9 nickase variants, the Cas9 proteins used herein may also include other “Cas9 variants” having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 protein, including any wild type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to a reference Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9

In some embodiments, the disclosure also may utilize Cas9 fragments which retain their functionality, and which are fragments of any herein disclosed Cas9 protein. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or 1300 amino acids in length.

In various embodiments, the prime editors disclosed herein may comprise one of the Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 variants.

### Small-Sized Cas9 Variants

In some embodiments, the prime editors contemplated herein can include a Cas9 protein that is of smaller molecular weight than the canonical SpCas9 sequence. In some embodiments, the smaller-sized Cas9 variants may facilitate delivery to cells, e.g., by an expression vector, nanoparticle, or other means of delivery. In certain embodiments, the smaller-sized Cas9 variants can include enzymes categorized as type II enzymes of the Class 2 CRISPR-Cas systems. In some embodiments, the smaller-sized Cas9 variants can include enzymes categorized as type V enzymes of the Class 2 CRISPR-Cas systems. In other embodiments, the smaller-sized Cas9 variants can include enzymes categorized as type VI enzymes of the Class 2 CRISPR-Cas systems.

The canonical SpCas9 protein is 1368 amino acids in length and has a predicted molecular weight of 158 kilodaltons. The term “small-sized Cas9 variant”, as used herein, refers to any Cas9 variant—naturally occurring, engineered, or otherwise—that is less than at least 1300 amino acids, or at least less than 1290 amino acids, or than less than 1280 amino acids, or less than 1270 amino acid, or less than 1260 amino acid, or less than 1250 amino acids, or less than 1240 amino acids, or less than 1230 amino acids, or less than 1220 amino acids, or less than 1210 amino acids, or less than 1200 amino acids, or less than 1190 amino acids, or less than 1180 amino acids, or less than 1170 amino acids, or less than 1160 amino acids, or less than 1150 amino acids, or less than 1140 amino acids, or less than 1130 amino acids, or less than 1120 amino acids, or less than 1110 amino acids, or less than 1100 amino acids, or less than 1050 amino acids, or less than 1000 amino acids, or less than 950 amino acids, or less than 900 amino acids, or less than 850 amino acids, or less than 800 amino acids, or less than 750 amino acids, or less than 700 amino acids, or less than 650 amino acids, or less than 600 amino acids, or less than 550 amino acids, or less than 500 amino acids, but at least larger than about 400 amino acids and retaining the required functions of the Cas9 protein. The Cas9 variants can include those categorized as type II, type V, or type VI enzymes of the Class 2 CRISPR-Cas system.

In various embodiments, the prime editors disclosed herein may comprise any small-sized Cas9 variants known in the art, or a Cas9 variant thereof. Exemplary embodiments include: SaCas9 (Staphylococcus aureus, 1053 AA, 123 kDa), NmeCas9 (N. meningitidis, 1083 AA, 124.5 kDa), CjCas9 (C. jejuni. 984 AA, 114.9 kDa), GeoCas9 (G. stearothermophilus, 1087 AA, 127 kDa), LbaCas12a (L. bacterium, 1228 AA, 143.9 kDa), BhCas12b (B. hisashii, 1108 AA, 130.4 kDa). Any amino acid sequence known in the art having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference small-sized Cas9 protein are herein contemplated.

### Cas9 Equivalents

In some embodiments, the prime editors described herein can include any Cas9 equivalent. As used herein, the term “Cas9 equivalent” is a broad term that encompasses any napDNAbp protein that serves the same function as Cas9 in the present prime editors despite that its amino acid primary sequence and/or its three-dimensional structure may be different and/or unrelated from an evolutionary standpoint. Thus, while Cas9 equivalents include any Cas9 ortholog, homolog, mutant, or variant described or embraced herein that are evolutionarily related, the Cas9 equivalents also embrace proteins that may have evolved through convergent evolution processes to have the same or similar function as Cas9, but which do not necessarily have any similarity with regard to amino acid sequence and/or three dimensional structure. The prime editors described here embrace any Cas9 equivalent that would provide the same or similar function as Cas9 despite that the Cas9 equivalent may be based on a protein that arose through convergent evolution. For instance, if Cas9 refers to a type II enzyme of the CRISPR-Cas system, a Cas9 equivalent can refer to a type V or type VI enzyme of the CRISPR-Cas system.

For example, Cas12e (CasX) is a Cas9 equivalent that reportedly has the same function as Cas9 but which evolved through convergent evolution. Thus, the Cas12e (CasX) protein described in Liu et al., “CasX enzymes comprises a distinct family of RNA-guided genome editors,” Nature. 2019, Vol. 566: 218-223, is contemplated to be used with the prime editors described herein. In addition, any variant or modification of Cas12e (CasX) is conceivable and within the scope of the present disclosure.

Cas9 is a bacterial enzyme that evolved in a wide variety of species. However, the Cas9 equivalents contemplated herein may also be obtained from archaea, which constitute a domain and kingdom of single-celled prokaryotic microbes different from bacteria.

In some embodiments, Cas9 equivalents may refer to Cas12e (CasX) or Cas12d (CasY), which have been described in, for example, Burstein et al., “New CRISPR-Cas systems from uncultivated microbes.” Cell Res. 2017 Feb. 21. doi: 10.1038/cr.2017.21, the entire contents of which is hereby incorporated by reference. Using genome-resolved metagenomics, a number of CRISPR-Cas systems were identified, including the first reported Cas9 in the archaeal domain of life. This divergent Cas9 protein was found in little-studied nanoarchaea as part of an active CRISPR-Cas system. In bacteria, two previously unknown systems were discovered, CRISPR-Cas12e and CRISPR-Cas12d, which are among the most compact systems yet discovered. In some embodiments, Cas9 refers to Cas12e, or a variant of Cas12e. In some embodiments, Cas9 refers to a Cas12d, or a variant of Cas12d. It should be appreciated that other RNA-guided DNA binding proteins may be used as a nucleic acid programmable DNA binding protein (napDNAbp), and are within the scope of this disclosure. Also see Liu et al., “CasX enzymes comprises a distinct family of RNA-guided genome editors,” Nature, 2019, Vol. 566: 218-223. Any of these Cas9 equivalents are contemplated.

In some embodiments, the Cas9 equivalent comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12e (CasX) or Cas12d (CasY) protein. In some embodiments, the napDNAbp is a naturally-occurring Cas12e (CasX) or Cas12d (CasY) protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a wild-type Cas moiety or any Cas moiety provided herein.

In various embodiments, the nucleic acid programmable DNA binding proteins include, without limitation, Cas9 (e.g., dCas9 and nCas9), Cas12e (CasX), Cas12d (CasY), Cas12a (Cpf1), Cas12b1 (C2c1), Cas13a (C2c2), Cas12c (C2c3), Argonaute, and Cas12b1. One example of a nucleic acid programmable DNA-binding protein that has different PAM specificity than Cas9 is Clustered Regularly Interspaced Short Palindromic Repeats from Prevotella and Francisella 1 (i.e, Cas12a (Cpf1)). Similar to Cas9, Cas12a (Cpf1) is also a Class 2 CRISPR effector, but it is a member of type V subgroup of enzymes, rather than the type II subgroup. It has been shown that Cas12a (Cpf1) mediates robust DNA interference with features distinct from Cas9. Cas12a (Cpf1) is a single RNA-guided endonuclease lacking tracrRNA, and it utilizes a T-rich protospacer-adjacent motif (T1′N, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells. Cpf1 proteins are known in the art and have been described previously, for example Yamano et al., “Crystal structure of Cpf1 in complex with guide RNA and target DNA.” Cell (165) 2016, p. 949-962; the entire contents of which is hereby incorporated by reference.

In still other embodiments, the Cas protein may include any CRISPR associated protein, including but not limited to, Cas12a, Cas12b1. Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or homologs thereof, or modified versions thereof, and preferably comprising a nickase mutation.

In various other embodiments, the napDNAbp can be any of the following proteins: a Cas9, a Cas12a (Cpf1), a Cas12e (CasX), a Cas12d (CasY), a Cas12b1 (C2c1), a Cas13a (C2c2), a Cas12c (C2c3), a GeoCas9, a CjCas9, a Cas12g, a Cas12h, a Cas12i, a Cas13b, a Cas13c, a Cas13d, a Cas14, a Csn2, an xCas9, an SpCas9-NG, a circularly permuted Cas9, or an Argonaute (Ago) domain, or a variant thereof.

Exemplary Cas9 equivalents can include the following: AsCas12a (previously known as Cpf1. Acidaminococcus sp. (strain BV3L6) UniProtKB U2UMQ6), AsCasI2a nickase (e.g., R1226A), LbCas12a (previously known as Cpf1, Lachnospiraceae bacterium GAM79, Ref Seq. WP_119623382.1), PcCas12a (previously known at Cpf1, Prevotella copri, Ref Seq. WP_119227726.1), ErCas12a (previously known at Cpf1. Eubacterium rectale, Ref Seq. WP_119223642.1), CsCasI2a (previously known at Cpf1 Clostridium sp. AF34-10BH, Ref Seq. WP_118538418.1), BhCas12b (Bacillus hisashii, Ref Seq. WP_095142515.1), ThCas12b (Thermomonas hydrothermalis, Ref Seq. WP_072754838), LsCas12b (Laceyella sacchari, WP_132221894). DtCas12b (Dsulfonatronum thiodismutans. WP_031386437).

The prime editors described herein may also comprise Cas12a (Cpf1) (dCpf1) variants that may be used as a guide nucleotide sequence-programmable DNA-binding protein domain. The Cas12a (Cpf1) protein has a RuvC-like endonuclease domain that is similar to the RuvC domain of Cas9 but does not have a HNH endonuclease domain, and the N-terminal of Cas12a (Cpf1) does not have the alfa-helical recognition lobe of Cas9. It was shown in Zetsche et al., Cell, 163, 759-771, 2015 (which is incorporated herein by reference) that, the RuvC-like domain of Cas12a (Cpf1) is responsible for cleaving both DNA strands and inactivation of the RuvC-like domain inactivates Cas12a (Cpf1) nuclease activity.

In some embodiments, the napDNAbp is a single effector of a microbial CRISPR-Cas system. Single effectors of microbial CRISPR-Cas systems include, without limitation. Cas9, Cas12a (Cpf1), Cas12b1 (C2c1), Cas13a (C2c2), and Cas12c (C2c3). Typically, microbial CRISPR-Cas systems are divided into Class 1 and Class 2 systems. Class 1 systems have multi-subunit effector complexes, while Class 2 systems have a single protein effector. For example, Cas9 and Cas12a (Cpf1) are Class 2 effectors. In addition to Cas9 and Cas12a (Cpf1), three distinct Class 2 CRISPR-Cas systems (Cas12b1, Cas13a, and Cas12c) have been described by Shmakov et al., “Discovery and Functional Characterization of Diverse Class 2 CRISPR Cas Systems”, Mol. Cell, 2015 Nov. 5; 60(3): 385-397, the entire contents of which are hereby incorporated by reference.

Effectors of two of the systems, Cas12b1 and Cas12c, contain RuvC-like endonuclease domains related to Cas12a. A third system, Cas13a contains an effector with two predicated HEPN RNase domains. Production of mature CRISPR RNA is tracrRNA-independent, unlike production of CRISPR RNA by Cas12b1. Cas12b1 depends on both CRISPR RNA and tracrRNA for DNA cleavage. Bacterial Cas13a has been shown to possess a unique RNase activity for CRISPR RNA maturation distinct from its RNA-activated single-stranded RNA degradation activity. These RNase functions are different from each other and from the CRISPR RNA-processing behavior of Cas12a. See, e.g., East-Seletsky, et al., “Two distinct RNase activities of CRISPR-Cas13a enable guide-RNA processing and RNA detection”, Nature, 2016 Oct. 13:538(7624):270-273, the entire contents of which are hereby incorporated by reference. In vitro biochemical analysis of Cas13a in Leptotrichia shahii has shown that Cas13a is guided by a single CRISPR RNA and can be programed to cleave ssRNA targets carrying complementary protospacers. Catalytic residues in the two conserved HEPN domains mediate cleavage. Mutations in the catalytic residues generate catalytically inactive RNA-binding proteins. See e.g., Abudayyeh et al., “C2c2 is a single-component programmable RNA-guided RNA-targeting CRISPR effector”, Science, 2016 Aug. 5; 353(6299), the entire contents of which are hereby incorporated by reference.

The crystal structure of Alicyclobaccillus acidoterrastris Cas12b1 (AacC2c1) has been reported in complex with a chimeric single-molecule guide RNA (sgRNA). See e.g., Liu et al., “C2c1-sgRNA Complex Structure Reveals RNA-Guided DNA Cleavage Mechanism”, Mol. Cell, 2017 Jan. 19; 65(2):310-322, the entire contents of which are hereby incorporated by reference. The crystal structure has also been reported in Alicyclobacillus acidoterrestris C2c1 bound to target DNAs as ternary complexes. See e.g., Yang et al., “PAM-dependent Target DNA Recognition and Cleavage by C2C1 CRISPR-Cas endonuclease”, Cell, 2016 Dec. 15:167(7):1814-1828, the entire contents of which are hereby incorporated by reference. Catalytically competent conformations of AacC2c1, both with target and non-target DNA strands, have been captured independently positioned within a single RuvC catalytic pocket, with C2c1-mediated cleavage resulting in a staggered seven-nucleotide break of target DNA. Structural comparisons between C2c1 ternary complexes and previously identified Cas9 and Cpf1 counterparts demonstrate the diversity of mechanisms used by CRISPR-Cas9 systems.

In some embodiments, the napDNAbp may be a C2c1, a C2c2, or a C2c3 protein. In some embodiments, the napDNAbp is a C2c1 protein. In some embodiments, the napDNAbp is a Cas13a protein. In some embodiments, the napDNAbp is a Cas12c protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12b1 (C2c1), Cas13a (C2c2), or Cas12c (C2c3) protein. In some embodiments, the napDNAbp is a naturally-occurring Cas12b1 (C2c1), Cas13a (C2c2), or Cas12c (C2c3) protein.

### Cas9 Circular Permutants

In various embodiments, the prime editors disclosed herein may comprise a circular permutant of Cas9.

The term “circularly permuted Cas9” or “circular permutant” of Cas9 or “CP-Cas9”) refers to any Cas9 protein, or variant thereof, that occurs or has been modify to engineered as a circular permutant variant, which means the N-terminus and the C-terminus of a Cas9 protein (e.g., a wild type Cas9 protein) have been topically rearranged. Such circularly permuted Cas9 proteins, or variants thereof, retain the ability to bind DNA when complexed with a guide RNA (gRNA). See, Oakes et al., “Protein Engineering of Cas9 for enhanced function,” Methods Enzymol, 2014, 546: 491-511 and Oakes et al., “CRISPR-Cas9 Circular Permutants as Programmable Scaffolds for Genome Modification,” Cell, Jan. 10, 2019, 176: 254-267, each of are incorporated herein by reference. The instant disclosure contemplates any previously known CP-Cas9 or use a new CP-Cas9 so long as the resulting circularly permuted protein retains the ability to bind DNA when complexed with a guide RNA (gRNA).

Any of the Cas9 proteins described herein, including any variant, ortholog, or naturally occurring Cas9 or equivalent thereof, may be reconfigured as a circular permutant variant.

In various embodiments, the circular permutants of Cas9 may have the following structure:
N-terminus-[original C-terminus]-[optional linker]-[original N-terminus]-C-terminus.

As an example, the present disclosure contemplates the following circular permutants of canonical S. pyogenes Cas9 (1368 amino acids of UniProtKB—Q99ZW2 (CAS9_STRP1) (numbering is based on the amino acid position in SEQ ID NO: 18)):
N-terminus-[1268-1368]-[optional linker]-[1-1267]-C-terminus;
N-terminus-[1168-1368]-[optional linker]-[1-1167]-C-terminus;
N-terminus-[1068-1368]-[optional linker]-[1-1067]-C-terminus;
N-terminus-[968-1368]-[optional linker]-[1-967]-C-terminus;
N-terminus-[868-1368]-[optional linker]-[1-867]-C-terminus;
N-terminus-[768-1368]-[optional linker]-[1-767]-C-terminus;
N-terminus-[668-1368]-[optional linker]-[1-667]-C-terminus;
N-terminus-[568-1368]-[optional linker]-[1-567]-C-terminus;
N-terminus-[468-1368]-[optional linker]-[1-467]-C-terminus;
N-terminus-[368-1368]-[optional linker]-[1-367]-C-terminus;
N-terminus-[268-1368]-[optional linker]-[1-267]-C-terminus;
N-terminus-[168-1368]-[optional linker]-[1-167]-C-terminus;
N-terminus-[68-1368]-[optional linker]-[1-67]-C-terminus; or
N-terminus-[10-1368]-[optional linker]-[1-9]-C-terminus, or the corresponding circular permutants of other Cas9 proteins (including other Cas9 orthologs, variants, etc).

In particular embodiments, the circular permutant Cas9 has the following structure (based on S. pyogenes Cas9 (1368 amino acids of UniProtKB—Q99ZW2 (CAS9_STRP1) (numbering is based on the amino acid position in SEQ ID NO: 18):
N-terminus-[102-1368]-[optional linker]-[1-101]-C-terminus;
N-terminus-[1028-1368]-[optional linker]-[1-1027]-C-terminus;
N-terminus-[1041-1368]-[optional linker]-[1-1043]-C-terminus;
N-terminus-[1249-1368]-[optional linker]-[1-1248]-C-terminus; or
N-terminus-[1300-1368]-[optional linker]-[1-1299]-C-terminus, or the corresponding circular permutants of other Cas9 proteins (including other Cas9 orthologs, variants, etc).

In still other embodiments, the circular permutant Cas9 has the following structure (based on S. pyogenes Cas9 (1368 amino acids of UniProtKB—Q99ZW2 (CAS9_STRP1) (numbering is based on the amino acid position in SEQ ID NO: 18):
N-terminus-[103-1368]-[optional linker]-[1-102]-C-terminus;
N-terminus-[1029-1368]-[optional linker]-[1-1028]-C-terminus;
N-terminus-[1042-1368]-[optional linker]-[1-1041]-C-terminus;
N-terminus-[1250-1368]-[optional linker]-[1-1249]-C-terminus; or
N-terminus-[1301-1368]-[optional linker]-[1-1300]-C-terminus, or the corresponding circular permutants of other Cas9 proteins (including other Cas9 orthologs, variants, etc.).

In some embodiments, the circular permutant can be formed by linking a C-terminal fragment of a Cas9 to an N-terminal fragment of a Cas9, either directly or by using a linker, such as an amino acid linker.

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment that is rearranged to the N-terminus, includes or corresponds to the C-terminal 30% or less of the amino acids of a Cas9. In some embodiments, the C-terminal fragment that is rearranged to the N-terminus, includes or corresponds to the C-terminal 30%, 29%, 28%, 27%. 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the amino acids of a Cas9. In some embodiments, the C-terminal fragment that is rearranged to the N-terminus, includes or corresponds to the C-terminal 410 residues or less of a Cas9. In some embodiments, the C-terminal portion that is rearranged to the N-terminus, includes or corresponds to the C-terminal 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas9. In some embodiments, the C-terminal portion that is rearranged to the N-terminus, includes or corresponds to the C-terminal 357, 341, 328, 120, or 69 residues of a Cas9.

In other embodiments, circular permutant Cas9 variants may be defined as a topological rearrangement of a Cas9 primary structure based on the following method: (a) selecting a circular permutant (CP) site corresponding to an internal amino acid residue of the Cas9 primary structure, which dissects the original protein into two halves: an N-terminal region and a C-terminal region; (b) modifying the Cas9 protein sequence (e.g., by genetic engineering techniques) by moving the original C-terminal region (comprising the CP site amino acid) to precede the original N-terminal region, thereby forming a new N-terminus of the Cas9 protein that now begins with the CP site amino acid residue. The CP site can be located in any domain of the Cas9 protein, including, for example, the helical-II domain, the RuvCIII domain, or the CTD domain. For example, the CP site may be located (relative the S. pyogenes Cas9 of SEQ ID NO: 18) at original amino acid residue 181, 199, 230, 270, 310, 1010, 1016, 1023, 1029, 1041, 1247, 1249, or 1282. Thus, once relocated to the N-terminus, original amino acid 181, 199, 230, 270, 310, 1010, 1016, 1023, 1029, 1041, 1247, 1249, or 1282 would become the new N-terminal amino acid. Nomenclature of these CP-Cas9 proteins may be referred to as Cas9-CP181, Cas9-CP199, Cas9-CP230, Cas9-CP270, Cas9-CP310, Cas9-CP1010, Cas9-CP1016. Cas9-CP1023, Cas9-CP1029, Cas9-CP1041, Cas9-CP1247, Cas9-CP1249, and Cas9-CP1282, respectively. Exemplary C-terminal fragments of Cas9, which may be rearranged to an N-terminus of Cas9, include: CP1012 C-terminal fragment, CP1028 C-terminal fragment, CP1041 C-terminal fragment, CP1249 C-terminal fragment, CP1300 C-terminal fragment. It should be appreciated that such C-terminal fragments of Cas9 are exemplary and are not meant to be limiting.

### Polymerase Domain

In some embodiments, the prime editors disclosed herein comprise a polymerase domain or a variant thereof (e.g., DNA-dependent DNA polymerase or RNA-dependent DNA polymerase, such as, reverse transcriptase). In some cases, the polymerase, or variant thereof, may be provided as a fusion protein with a napDNAbp or other programmable nuclease, or provided in trans.

Any polymerase known in the art may be used in the prime editors with the methods and compositions disclosed herein. The polymerases may be wild type polymerases, functional fragments, mutants, variants, or truncated variants, and the like. The polymerases may include wild type polymerases from eukaryotic, prokaryotic, archael, or viral organisms, and/or the polymerases may be modified by genetic engineering, mutagenesis, or directed evolution-based processes. The polymerases may include 17 DNA polymerase, T5 DNA polymerase, T4 DNA polymerase, Klenow fragment DNA polymerase, DNA polymerase III and the like. The polymerases may also be thermostable, and may include Taq, Tne, Tma, Pfu, Tfl, Tth, Stoffel fragment, VENT® and DEEPVENT® DNA polymerases, KOD, Tgo, JDF3, and mutants, variants and derivatives thereof (see U.S. Pat. Nos. 5,436,149; 4,889,818; 4,965,185; 5,079,352; 5,614,365; 5,374,553; 5,270,179; 5,047,342; 5,512,462; WO 92/06188; WO 92/06200; WO 96/10640; Barnes, W. M., Gene 112:29-35 (1992); Lawyer, F. C., et al., PCR Meth. Appl. 2:275-287 (1993); Flaman, J.-M, et al., Nuc. Acids Res. 22(15):3259-3260 (1994), each of which are incorporated by reference).

In some embodiments, the polymerases used in the methods and compositions disclosed herein are “template-dependent” polymerase (since the polymerases are intended to rely on the DNA synthesis template to specify the sequence of the DNA strand under synthesis during prime editing. As used herein, the term “template DNA molecule” refers to that strand of a nucleic acid from which a complementary nucleic acid strand is synthesized by a DNA polymerase, for example, in a primer extension reaction of the DNA synthesis template of a PegRNA.

The disclosure contemplates any wild type polymerase obtained from any naturally-occurring organism or virus, or obtained from a commercial or non-commercial source. In addition, the polymerases usable in the prime editors can include any naturally-occurring mutant polymerase, engineered mutant polymerase, or other variant polymerase, including truncated variants that retain function. The polymerases usable herein may also be engineered to contain specific amino acid substitutions, such as those specifically disclosed herein. In certain preferred embodiments, the polymerases usable in the prime editors utilized in the methods and compositions of the present disclosure are template-based polymerases, i.e., they synthesize nucleotide sequences in a template-dependent manner.

In some embodiments, the polymerase is a DNA polymerase (e.g., a “DNA-dependent DNA polymerase” whereby the template molecule is a strand of DNA). In some embodiments, the polymerase is an RNA polymerase. In various other embodiments, the DNA polymerase can be an “RNA-dependent DNA polymerase” (i.e., whereby the template molecule is a strand of RNA). The term “polymerase” may also refer to an enzyme that catalyzes the polymerization of nucleotide (i.e., the polymerase activity). Generally, the enzyme will initiate synthesis at the 3-end of a primer annealed to a polynucleotide template sequence (e.g., such as a primer sequence annealed to the primer binding site of a PegRNA), and will proceed toward the 5′ end of the template strand.

In some embodiments, the DNA polymerase is a “functional fragment thereof”. A “functional fragment thereof” refers to any portion of a wild-type or mutant DNA polymerase that encompasses less than the entire amino acid sequence of the polymerase and which retains the ability, under at least one set of conditions, to catalyze the polymerization of a polynucleotide. Such a functional fragment may exist as a separate entity, or it may be a constituent of a larger polypeptide, such as a fusion protein.

In some embodiments, the polymerase is a reverse transcriptase (RT). RTs are art recognized enzymes with RNA- and DNA-dependent DNA polymerization activity, and an RNaseH activity that catalyzes the cleavage of RNA in RNA-DNA hybrids. In some embodiments, the RT is mutated to disable the RNaseH domain (e.g., to prevent unintended damage to the mRNA). In other embodiments, still, the RNaseH domain is truncated.

Any of the wild type, variant, and/or mutant forms of reverse transcriptases known in the art or which can be made using methods known in the art, such as those described by [[U.S. patents XXXX]]], are contemplated herein. For example, in some embodiments, the RT is a wild type RT. Non-limiting examples of RTs include Moloney Murine Leukemia Virus (M-MLV); Human Immunodeficiency Virus (HIV) reverse transcriptase and avian Sarcoma-Leukosis Virus (ASLV) reverse transcriptase, which includes but is not limited to Rous Sarcoma Virus (RSV) reverse transcriptase, Avian Myeloblastosis Virus (AMV) reverse transcriptase, Avian Erythroblastosis Virus (AEV) Helper Virus MCAV reverse transcriptase. Avian Myelocytomatosis Virus MC29 Helper Virus MCAV reverse transcriptase, Avian Reticuloendotheliosis Virus (REV-T) Helper Virus REV-A reverse transcriptase, Avian Sarcoma Virus UR2 Helper Virus UR2AV reverse transcriptase, Avian Sarcoma Virus Y73 Helper Virus YAV reverse transcriptase, Rous Associated Virus (RAV) reverse transcriptase, and Myeloblastosis Associated Virus (MAV).

pegRNAs

In some embodiments, the compositions and methods for prime editing contemplated herein comprise at least one pegRNA. Any suitable pegRNA architecture known in the art may be used in any one of the compositions and methods for prime editing disclosed herein, such as those described in U.S. Provisional Application U.S. Ser. No. 63/255,897, U.S. Provisional Application U.S. Ser. No. 63/231,230, U.S. Provisional Application U.S. Ser. No. 63/194,913, U.S. Provisional Application U.S. Ser. No. 63/194,865, U.S. Provisional Application U.S. Ser. No. 63/176,180, U.S. Provisional Application U.S. Ser. No. 63/176,202, and U.S. Provisional Application U.S. Ser. No. 63/136,194, U.S. Provisional Application No. 63/022,397, U.S. Provisional Application No. 63/116,785, International Patent Application No. PCT/US2021/031439, and International Patent Application No. PCT/US2022/012054, the entire contents each of which is incorporated herein by reference in their entireties.

In some embodiments, the pegRNA comprises a spacer sequence, gRNA core, a DNA synthesis template, and a primer binding site. As used herein, the term “spacer sequence” in connection with a guide RNA or a pegRNA refers to the portion of the guide RNA or pegRNA of about 20 nucleotides which contains a nucleotide sequence that shares the same sequence as the protospacer sequence in the target DNA sequence. The spacer sequence anneals to the complement of the protospacer sequence to form a ssRNA/ssDNA hybrid structure at the target site and a corresponding R loop ssDNA structure of the endogenous DNA strand.

In some embodiments, the pegRNA comprises a gRNA core.

In some embodiments, an extended guide RNA usable in the prime editing system utilized in the methods and compositions disclosed herein whereby a traditional guide RNA includes a ˜20 nt protospacer sequence and a gRNA core region, which binds with the napDNAbp. In this embodiment, the guide RNA includes an extended RNA segment at the 5′ end, i.e., a 5′ extension. In this embodiment, the 5′extension includes a reverse transcription template sequence, a reverse transcription primer binding site, and an optional 5-20 nucleotide linker sequence. The RT primer binding site hybridizes to the free 3′ end that is formed after a nick is formed in the non-target strand of the R-loop, thereby priming reverse transcriptase for DNA polymerization in the 5′-3′ direction.

In another embodiment, an extended guide RNA usable in the prime editing system utilized in the methods and compositions disclosed herein whereby a traditional guide RNA includes a ˜20 nt protospacer sequence and a gRNA core, which binds with the napDNAbp. In this embodiment, the guide RNA includes an extended RNA segment at the 3′ end, i.e., a 3′ extension. In this embodiment, the 3′extension includes a reverse transcription template sequence, and a reverse transcription primer binding site. The RT primer binding site hybridizes to the free 3′ end that is formed after a nick is formed in the non-target strand of the R-loop, thereby priming reverse transcriptase for DNA polymerization in the 5′-3′ direction.

In another embodiment, an extend guide RNA usable in the prime editing system utilized in the methods and compositions disclosed herein whereby a traditional guide RNA includes a ˜20 nt protospacer sequence and a gRNA core, which binds with the napDNAbp. In this embodiment, the guide RNA includes an extended RNA segment at an intermolecular position within the gRNA core, i.e., an intramolecular extension. In this embodiment, the intramolecular extension includes a reverse transcription template sequence, and a reverse transcription primer binding site. The RT primer binding site hybridizes to the free 3′ end that is formed after a nick is formed in the non-target strand of the R-loop, thereby priming reverse transcriptase for DNA polymerization in the 5′-3′ direction.

In one embodiment, the position of the intermolecular RNA extension is not in the protospacer sequence of the guide RNA. In another embodiment, the position of the intermolecular RNA extension in the gRNA core. In still another embodiment, the position of the intermolecular RNA extension is any with the guide RNA molecule except within the protospacer sequence, or at a position which disrupts the protospacer sequence.

In one embodiment, the intermolecular RNA extension is inserted downstream from the 3′ end of the protospacer sequence. In another embodiment, the intermolecular RNA extension is inserted at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides downstream of the 3′ end of the protospacer sequence.

In other embodiments, the intermolecular RNA extension is inserted into the gRNA, which refers to the portion of the guide RNA corresponding or comprising the tracrRNA, which binds and/or interacts with the Cas9 protein or equivalent thereof (i.e. a different napDNAbp). Preferably the insertion of the intermolecular RNA extension does not disrupt or minimally disrupts the interaction between the tracrRNA portion and the napDNAbp.

The length of the RNA extension (which includes at least the RT template and primer binding site) can be any useful length. In various embodiments, the RNA extension is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

The RT template sequence can also be any suitable length. For example, the RT template sequence can be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In still other embodiments, wherein the reverse transcription primer binding site sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In other embodiments, the optional linker or spacer sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

The RT template sequence, in certain embodiments, encodes a single-stranded DNA molecule which is homologous to the non-target strand (and thus, complementary to the corresponding site of the target strand) but includes one or more nucleotide changes. The least one nucleotide change may include one or more single-base nucleotide changes, one or more deletions, and one or more insertions.

The synthesized single-stranded DNA product of the RT template sequence is homologous to the non-target strand and contains one or more nucleotide changes. The single-stranded DNA product of the RT template sequence hybridizes in equilibrium with the complementary target strand sequence, thereby displacing the homologous endogenous target strand sequence. The displaced endogenous strand may be referred to in some embodiments as a 5′ endogenous DNA flap species. This 5′ endogenous DNA flap species can be removed by a 5′ flap endonuclease (e.g., FEN1) and the single-stranded DNA product, now hybridized to the endogenous target strand, may be ligated, thereby creating a mismatch between the endogenous sequence and the newly synthesized strand. The mismatch may be resolved by the cell's innate DNA repair and/or replication processes.

In various embodiments, the nucleotide sequence of the RT template sequence corresponds to the nucleotide sequence of the non-target strand which becomes displaced as the 5′ flap species and which overlaps with the site to be edited.

In various embodiments of the extended guide RNAs, the reverse transcription template sequence may encode a single-strand DNA flap that is complementary to an endogenous DNA sequence adjacent to a nick site, wherein the single-strand DNA flap comprises a desired nucleotide change. The single-stranded DNA flap may displace an endogenous single-strand DNA at the nick site. The displaced endogenous single-strand DNA at the nick site can have a 5′ end and form an endogenous flap, which can be excised by the cell. In various embodiments, excision of the 5′ end endogenous flap can help drive product formation since removing the 5′ end endogenous flap encourages hybridization of the single-strand 3′ DNA flap to the corresponding complementary DNA strand, and the incorporation or assimilation of the desired nucleotide change carried by the single-strand 3′ DNA flap into the target DNA.

In various embodiments of the extended guide RNAs, the cellular repair of the single-strand DNA flap results in installation of the desired nucleotide change, thereby forming a desired product.

In still other embodiments, the desired nucleotide change is installed in an editing window that is between about −5 to +5 of the nick site, or between about −10 to +10 of the nick site, or between about −20 to +20 of the nick site, or between about −30 to +30 of the nick site, or between about −40 to +40 of the nick site, or between about −50 to +50 of the nick site, or between about −60 to +60 of the nick site, or between about −70 to +70 of the nick site, or between about −80 to +80 of the nick site, or between about −90 to +90 of the nick site, or between about −100 to +100 of the nick site, or between about −200 to +200 of the nick site.

In other embodiments, the desired nucleotide change is installed in an editing window that is between about +1 to +2 from the nick site, or about +1 to +3, +1 to +4, +1 to +5, +1 to +6, +1 to +7, +1 to +8, +1 to +9, +1 to +10, +1 to +11, +1 to +12, +1 to +13, +1 to +14, +1 to +15, +1 to +16, +1 to +17, +1 to +18, +1 to +19, +1 to +20, +1 to +21, +1 to +22, +1 to +23, +1 to +24, +1 to +25, +1 to +26, +1 to +27, +1 to +28, +1 to +29, +1 to +30, +1 to +31, +1 to +32, +1 to +33, +1 to +34, +1 to +35, +1 to +36, +1 to +37, +1 to +38, +1 to +39, +1 to +40, +1 to +41, +1 to +42, +1 to +43, +1 to +44, +1 to +45, +1 to +46, +1 to +47, +1 to +48, +1 to +49, +1 to +50, +1 to +51, +1 to +52, +1 to +53, +1 to +54, +1 to +55, +1 to +56, +1 to +57, +1 to +58, +1 to +59, +1 to +60, +1 to +61, +1 to +62, +1 to +63, +1 to +64, +1 to +65, +1 to +66, +1 to +67, +1 to +68, +1 to +69, +1 to +70, +1 to +71, +1 to +72, +1 to +73, +1 to +74, +1 to +75, +1 to +76, +1 to +77, +1 to +78, +1 to +79, +1 to +80, +1 to +81, +1 to +82, +1 to +83, +1 to +84, +1 to +85, +1 to +86, +1 to +87, +1 to +88, +1 to +89, +1 to +90, +1 to +90, +1 to +91, +1 to +92, +1 to +93, +1 to +94, +1 to +95, +1 to +96, +1 to +97, +1 to +98, +1 to +99, +1 to +100, +1 to +101, +1 to +102, +1 to +103, +1 to +104, +1 to +105, +1 to +106, +1 to +107, +1 to +108, +1 to +109, +1 to +110, +1 to +111, +1 to +112, +1 to +113, +1 to +114, +1 to +115, +1 to +116, +1 to +117, +1 to +118, +1 to +119, +1 to +120, +1 to +121, +1 to +122, +1 to +123, +1 to +124, or +1 to +125 from the nick site.

In still other embodiments, the desired nucleotide change is installed in an editing window that is between about +1 to +2 from the nick site, or about +1 to +5, +1 to +10, +1 to +15, +1 to +20, +1 to +25, +1 to +30, +1 to +35, +1 to +40, +1 to +45, +1 to +50, +1 to +55, +1 to +100, +1 to +105, +1 to +110, +1 to +115, +1 to +120, +1 to +125, +1 to +130, +1 to +135, +1 to +140, +1 to +145, +1 to +150, +1 to +155, +1 to +160, +1 to +165, +1 to +170, +1 to +175, +1 to +180, +1 to +185, +1 to +190, +1 to +195, or +1 to +200, from the nick site.

In various aspects, the extended guide RNAs are modified versions of a guide RNA. Guide RNAs maybe naturally occurring, expressed from an encoding nucleic acid, or synthesized chemically. Methods are well known in the art for obtaining or otherwise synthesizing guide RNAs and for determining the appropriate sequence of the guide RNA, including the protospacer sequence which interacts and hybridizes with the target strand of a genomic target site of interest.

In various embodiments, the particular design aspects of a guide RNA sequence will depend upon the nucleotide sequence of a genomic target site of interest (i.e., the desired site to be edited) and the type of napDNAbp (e.g., Cas9 protein) present in the prime editing systems utilized in the methods and compositions described herein, among other factors, such as PAM sequence locations, percent G/C content in the target sequence, the degree of microhomology regions, secondary structures, etc.

In general, a guide sequence is any polynucleotide sequence having sufficient complementarity with a target polynucleotide sequence to hybridize with the target sequence and direct sequence-specific binding of a napDNAbp (e.g., a Cas9, Cas9 homolog, or Cas9 variant) to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length.

In some embodiments, a guide sequence is less than about 75, 50, 45, 40, 35, 30, 25, 20, 15, 12, or fewer nucleotides in length. The ability of a guide sequence to direct sequence-specific binding of a prime editor to a target sequence may be assessed by any suitable assay. For example, the components of a prime editor, including the guide sequence to be tested, may be provided to a host cell having the corresponding target sequence, such as by transfection with vectors encoding the components of a prime editor disclosed herein, followed by an assessment of preferential cleavage within the target sequence, such as by Surveyor assay as described herein. Similarly, cleavage of a target polynucleotide sequence may be evaluated in a test tube by providing the target sequence, components of a prime editor, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMMNNNNNNNNNNNNXGG (SEQ ID NO: 29) where NNNNNNNNNNNNXGG (SEQ ID NO: 30) (N is A, G. T, or C; and X can be anything). A unique target sequence in a genome may include an S. pyogenes Cas9 target site of the form MMMMMMMMMNNNNNNNNNNNXGG (SEQ ID NO: 31) where NNNNNNNNNNNXGG (SEQ ID NO: 32) (N is A, G, T, or C; and X can be anything). For the S. thermophilus CRISPR1Cas9, a unique target sequence in a genome may include a Cas9 target site of the form MMMMMMMMNNNNNNNNNNNNXXAGAAW (SEQ ID NO: 33) where NNNNNNNNNNNNXXAGAAW (SEQ ID NO: 34) (N is A, G, T, or C; X can be anything; and W is A or T). A unique target sequence in a genome may include an S. thermophilus CRISPR 1 Cas9 target site of the form MMMMMMMMMNNNNNNNNNNNXXAGAAW (SEQ ID NO: 35) where NNNNNNNNNNNXXAGAAW (SEQ ID NO: 36) (N is A, G, T, or C; X can be anything; and W is A or T). For the S. pyogenes Cas9, a unique target sequence in a genome may include a Cas9 target site of the form MMMMMMMMNNNNNNNNNNNNXGGXG (SEQ ID NO: 37) where NNNNNNNNNNNNXGGXG (SEQ ID NO: 38) (N is A, G, T, or C; and X can be anything). A unique target sequence in a genome may include an S. pyogenes Cas9 target site of the form MMMMMMMMMNNNNNNNNNNNXGGXG (SEQ ID NO: 39) where NNNNNNNNNNNXGGXG (SEQ ID NO: 40) (N is A, G, T, or C; and X can be anything). In each of these sequences “M” may be A, G. T, or C, and need not be considered in identifying a sequence as unique.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A. R. Gruber et al., 2008, Cell 106(1): 23-24; and PA Carr and GM Church, 2009, Nature Biotechnology 27(12): 1151-62). Further algorithms may be found in U.S. application Ser. No. 61/836,080; Broad Reference BI-2013/004A); incorporated herein by reference.

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a complex at a target sequence, wherein the complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In some embodiments, the single transcript further includes a transcription termination sequence; preferably this is a polyT sequence, for example six T nucleotides. Further non-limiting examples of single polynucleotides comprising a guide sequence, a tracr mate sequence, and a tracr sequence are as follows (listed 5′ to 3′), where “N” represents a base of a guide sequence, the first block of lower case letters represent the tracr mate sequence, and the second block of lower case letters represent the tracr sequence, and the final poly-T sequence represents the transcription terminator:

(1)
(SEQ ID NO: 41)
NNNNNNNNGTTTTTGTACTCTCAAGATTTAGAAATAAATCTTGCA
GAAGCTACAAAGATAAGGCTTCATGCCGAAATCAACACCCTGTCA
TTTTATGGCAGGGTGTTTTCGTTATTTAATTTTTT;
(2)
(SEQ ID NO: 42)
NNNNNNNNNNNNNNNNNNGTTTTTGTACTCTCAGAAATGCAGAAG
CTACAAAGATAAGGCTTCATGCCGAAATCAACACCCTGTCATTTT
ATGGCAGGGTGTTTTCGTTATTTAATTTTTT;
(3)
(SEQ ID NO: 43)
NNNNNNNNNNNNNNNNNNNNGTTTTTGTACTCTCAGAAATGCAGA
AGCTACAAAGATAAGGCTTCATGCCGAAATCAACACCCTGTCATT
TTATGGCAGGGTGTTTTTT;
(4)
(SEQ ID NO: 44)
NNNNNNNNNNNNNNNNNNNNGTTTTAGAGCTAGAAATAGCAAGTT
AAAATAAGGCTAGTCCGTTATCAACTTGAAAAAGTGGCACCGAGT
CGGTGCTTTTTT;
(5)
(SEQ ID NO: 45)
NNNNNNNNNNNNNNNNNNNNGTTTTAGAGCTAGAAATAGCAAGTT
AAAATAAGGCTAGTCCGTTATCAACTTGAAAAAGTGTTTTTTT;
and
(6)
(SEQ ID NO: 46)
NNNNNNNNNNNNNNNNNNNNGTTTTAGAGCTAGAAATAGCAAGTT
AAAATAAGGCTAGTCCGTTATCATTTTTTTT.

In some embodiments, sequences (1) to (3) are used in combination with Cas9 from S. thermophilus CRISPR1. In some embodiments, sequences (4) to (6) are used in combination with Cas9 from S. pyogenes. In some embodiments, the tracr sequence is a separate transcript from a transcript comprising the tracr mate sequence.

It will be apparent to those of skill in the art that in order to target any of the fusion proteins comprising a Cas9 domain and a single-stranded DNA binding protein, as disclosed herein, to a target site, e.g., a site comprising a point mutation to be edited, it is typically necessary to co-express the fusion protein together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9:nucleic acid editing enzyme/domain fusion protein.

In some embodiments, the guide RNA comprises a structure 5′-[guide sequence]-GUUUUAGAGCUAGAAAUAGCAAGUUAAAAUAAAGGCUAGUCCGUUAUCAACU UGAAAAAGUGGCACCGAGUCGGUGCUUUUU-3′ (SEQ ID NO: 47), wherein the guide sequence comprises a sequence that is complementary to the target sequence. The guide sequence is typically 20 nucleotides long. The sequences of suitable guide RNAs for targeting Cas9:nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein. Additional guide sequences are well known in the art and can be used with the prime editors utilized in the methods and compositions described herein.

In some embodiments, a pegRNA comprises three main component elements ordered in the 5′ to 3′ direction, namely: a spacer, a gRNA core, and an extension arm at the 3′ end. The extension arm may further be divided into the following structural elements in the 5′ to 3′ direction, namely: a primer binding site (A), an edit template (B), and a homology arm (C). In addition, the pegRNA may comprise an optional 3′ end modifier region (e1) and an optional 5′ end modifier region (e2). Still further, the pegRNA may comprise a transcriptional termination signal at the 3′ end of the pegRNA (not depicted). These structural elements are further defined herein. The depiction of the structure of the pegRNA is not meant to be limiting and embraces variations in the arrangement of the elements. For example, the optional sequence modifiers (e1) and (e2) could be positioned within or between any of the other regions shown, and not limited to being located at the 3′ and 5′ ends.

In some embodiments, a pegRNA contemplated herein and may be designed in accordance with the methodology defined in Example 2. The pegRNA comprises three main component elements ordered in the 5′ to 3′ direction, namely: a spacer, a gRNA core, and an extension arm at the 3′ end. The extension arm may further be divided into the following structural elements in the 5′ to 3′ direction, namely: a primer binding site (A), an edit template (B), and a homology arm (C). In addition, the pegRNA may comprise an optional 3′ end modifier region (e1) and an optional 5′ end modifier region (e2). Still further, the pegRNA may comprise a transcriptional termination signal on the 3′ end of the pegRNA (not depicted). These structural elements are further defined herein. The depiction of the structure of the pegRNA is not meant to be limiting and embraces variations in the arrangement of the elements. For example, the optional sequence modifiers (e1) and (e2) could be positioned within or between any of the other regions shown, and not limited to being located at the 3′ and 5′ ends.

The pegRNAs may also include additional design improvements that may modify the properties and/or characteristics of pegRNAs thereby improving the efficacy of prime editing. In various embodiments, these improvements may belong to one or more of a number of different categories, including but not limited to: (1) designs to enable efficient expression of functional pegRNAs from non-polymerase III (pol III) promoters, which would enable the expression of longer pegRNAs without burdensome sequence requirements: (2) improvements to the core, Cas9-binding pegRNA scaffold, which could improve efficacy; (3) modifications to the pegRNA to improve RT processivity, enabling the insertion of longer sequences at targeted genomic loci; and (4) addition of RNA motifs to the 5′ or 3′ termini of the pegRNA that improve pegRNA stability, enhance RT processivity, prevent misfolding of the pegRNA, or recruit additional factors important for genome editing.

In one embodiment, pegRNA could be designed with polIII promoters to improve the expression of longer-length pegRNA with larger extension arms. sgRNAs are typically expressed from the U6 snRNA promoter. This promoter recruits pol III to express the associated RNA and is useful for expression of short RNAs that are retained within the nucleus. However, pol T is not highly processive and is unable to express RNAs longer than a few hundred nucleotides in length at the levels required for efficient genome editing. Additionally, pol III can stall or terminate at stretches of U's, potentially limiting the sequence diversity that could be inserted using a pegRNA. Other promoters that recruit polymerase II (such as pCMV) or polymerase I (such as the U1 snRNA promoter) have been examined for their ability to express longer sgRNAs. However, these promoters are typically partially transcribed, which would result in extra sequence 5′ of the spacer in the expressed pegRNA, which has been shown to result in markedly reduced Cas9:sgRNA activity in a site-dependent manner. Additionally, while pol III-transcribed pegRNAs can simply terminate in a run of 6-7 U's, pegRNAs transcribed from pol 1H or pol I would require a different termination signal. Often such signals also result in polyadenylation, which would result in undesired transport of the pegRNA from the nucleus. Similarly, RNAs expressed from pol II promoters such as pCMV are typically 5′-capped, also resulting in their nuclear export.

Previously, Rinn and coworkers screened a variety of expression platforms for the production of long-noncoding RNA- (lncRNA) tagged sgRNAs183. These platforms include RNAs expressed from pCMV and that terminate in the ENE element from the MALAT1 ncRNA from humans¹⁸⁴, the PAN ENE element from KSHV¹⁸⁵, or the 3′ box from U1 snRNA¹⁸⁶. Notably, the MALAT1 ncRNA and PAN ENEs form triple helices protecting the polyA-tail^{184,187}. These constructs could also enhance RNA stability. It is contemplated that these expression systems will also enable the expression of longer pegRNAs.

In addition, a series of methods have been designed for the cleavage of the portion of the pol II promoter that would be transcribed as part of the pegRNA, adding either a self-cleaving ribozyme such as the hammerhead¹⁸⁸, pistol¹⁸⁹, hatchet¹⁸⁹, hairpin¹⁹⁰, VS¹⁹¹, twister¹⁹², or twister sister¹⁹² ribozymes, or other self-cleaving elements to process the transcribed guide, or a hairpin that is recognized by Csy4¹⁹³ and also leads to processing of the guide. Also, it is hypothesized that incorporation of multiple ENE motifs could lead to improved pegRNA expression and stability, as previously demonstrated for the KSHV PAN RNA and element¹⁸⁵. It is also anticipated that circularizing the pegRNA in the form of a circular intronic RNA (ciRNA) could also lead to enhanced RNA expression and stability, as well as nuclear localization¹⁹⁴.

The core, Cas9-binding pegRNA scaffold can likely be improved to enhance PE activity. Several such approaches have already been demonstrated. For instance, the first pairing element of the scaffold (P1) contains a GTTTT-AAAAC pairing element. Such runs of Ts have been shown to result in pol III pausing and premature termination of the RNA transcript. Rational mutation of one of the T-A pairs to a G-C pair in this portion of P1 has been shown to enhance sgRNA activity, suggesting this approach would also be feasible for pegRNAs¹⁹⁵. Additionally, increasing the length of P1 has also been shown to enhance sgRNA folding and lead to improved activity¹⁹⁵, suggesting it as another avenue for the improvement of pegRNA activity

In various other embodiments, the pegRNA may be improved by introducing modifications to the edit template region. As the size of the insertion templated by the pegRNA increases, it is more likely to be degraded by endonucleases, undergo spontaneous hydrolysis, or fold into secondary structures unable to be reverse-transcribed by the RT or that disrupt folding of the pegRNA scaffold and subsequent Cas9-RT binding. Accordingly, it is likely that modification to the template of the pegRNA might be necessary to affect large insertions, such as the insertion of whole genes. Some strategies to do so include the incorporation of modified nucleotides within a synthetic or semi-synthetic pegRNA that render the RNA more resistant to degradation or hydrolysis or less likely to adopt inhibitory secondary structures196. Such modifications could include 8-aza-7-deazaguanosine, which would reduce RNA secondary structure in G-rich sequences; locked-nucleic acids (LNA) that reduce degradation and enhance certain kinds of RNA secondary structure; 2′-O-methyl, 2′-fluoro, or 2′-O-methoxyethoxy modifications that enhance RNA stability. Such modifications could also be included elsewhere in the pegRNA to enhance stability and activity. Alternatively or additionally, the template of the pegRNA could be designed such that it both encodes for a desired protein product and is also more likely to adopt simple secondary structures that are able to be unfolded by the RT. Such simple structures would act as a thermodynamic sink, making it less likely that more complicated structures that would prevent reverse transcription would occur. Finally, one could also split the template into two, separate pegRNAs. In such a design, a PE would be used to initiate transcription and also recruit a separate template RNA to the targeted site via an RNA-binding protein fused to Cas9 or an RNA recognition element on the pegRNA itself such as the MS2 aptamer. The RT could either directly bind to this separate template RNA, or initiate reverse transcription on the original pegRNA before swapping to the second template. Such an approach could enable long insertions by both preventing misfolding of the pegRNA upon addition of the long template and also by not requiring dissociation of Cas9 from the genome for long insertions to occur, which could possibly be inhibiting PE-based long insertions.

In still other embodiments, the pegRNA may be improved by introducing additional RNA motifs at the 5′ and 3′ termini of the pegRNAs, or even at positions therein between (e.g., in the gRNA core region, or the spacer). Several such motifs—such as the PAN ENE from KSHV and the ENE from MALAT1 were discussed above as possible means to terminate expression of longer pegRNAs from non-pol III promoters. These elements form RNA triple helices that engulf the polyA tail, resulting in their being retained within the nucleus^{184, 187}. However, by forming complex structures at the 3′ terminus of the pegRNA that occlude the terminal nucleotide, these structures would also likely help prevent exonuclease-mediated degradation of pegRNAs.

Other structural elements inserted at the 3′ terminus could also enhance RNA stability, albeit without enabling termination from non-pol III promoters. Such motifs could include hairpins or RNA quadruplexes that would occlude the 3′ terminus¹⁹⁷, or self-cleaving ribozymes such as HDV that would result in the formation of a 2′-3′-cyclic phosphate at the 3′ terminus and also potentially render the pegRNA less likely to be degraded by exonucleases¹⁹⁸. Inducing the pegRNA to cyclize via incomplete splicing—to form a ciRNA—could also increase pegRNA stability and result in the pegRNA being retained within the nucleus.¹⁹⁴.

Additional RNA motifs could also improve RT processivity or enhance pegRNA activity by enhancing RT binding to the DNA-RNA duplex. Addition of the native sequence bound by the RT in its cognate retroviral genome could enhance RT activity¹⁹⁹. This could include the native primer binding site (PBS), polypurine tract (PPT), or kissing loops involved in retroviral genome dimerization and initiation of transcription¹⁹⁹.

Addition of dimerization motifs—such as kissing loops or a GNRA tetraloop/tetraloop receptor pair²⁰⁰—at the 5′ and 3′ termini of the pegRNA could also result in effective circularization of the pegRNA, improving stability. Additionally, it is envisioned that addition of these motifs could enable the physical separation of the pegRNA spacer and primer, prevention occlusion of the spacer which would hinder PE activity. Short 5′ extensions or 3′ extensions to the pegRNA that form a small toehold hairpin in the spacer region or along the primer binding site could also compete favorably against the annealing of intra-complementary regions along the length of the pegRNA, e.g., the interaction between the spacer and the primer binding site that can occur. Finally, kissing loops could also be used to recruit other template RNAs to the genomic site and enable swapping of RT activity from one RNA to the other.

pegRNA scaffolds could be further improved via directed evolution, in an analogous fashion to how SpCas9 and prime editors (PE) have been improved. Directed evolution could enhance pegRNA recognition by Cas9 or evolved Cas9 variants. Additionally, it is likely that different pegRNA scaffold sequences would be optimal at different genomic loci, either enhancing PE activity at the site in question, reducing off-target activities, or both. Finally, evolution of pegRNA scaffolds to which other RNA motifs have been added would almost certainly improve the activity of the fused pegRNA relative to the unevolved, fusion RNA. For instance, evolution of allosteric ribozymes composed of c-di-GMP-I aptamers and hammerhead ribozymes led to dramatically improved activity²⁰², suggesting that evolution would improve the activity of hammerhead-pegRNA fusions as well. In addition, while Cas9 currently does not generally tolerate 5′ extension of the sgRNA, directed evolution will likely generate enabling mutations that mitigate this intolerance, allowing additional RNA motifs to be utilized.

The present disclosure contemplates any such ways to further improve the efficacy of the prime editing systems utilized in the methods and compositions disclosed here.

In various embodiments, it may be advantageous to limit the appearance of consecutive sequence of Ts from the extension arm as consecutive series of T's may limit the capacity of the pegRNA to be transcribed. For example, strings of at least consecutive three T's, at least consecutive four T's, at least consecutive live T's, at least consecutive six T's, at least consecutive seven T's, at least consecutive eight T's, at least consecutive nine T's, at least consecutive ten T's, at least consecutive eleven T's, at least consecutive twelve T's, at least consecutive thirteen T's, at least consecutive fourteen T's, or at least consecutive fifteen T's should be avoided when designing the pegRNA, or should be at least removed from the final designed sequence. In one embodiment, one can avoid the includes of unwanted strings of consecutive T's in pegRNA extension arms but avoiding target sites that are rich in consecutive A:T nucleobase pairs.

In some embodiments, the pegRNA comprises one of the following architectures:
(i) 5′-[spacer]-[crRNA scaffold]-[RT template and PBS]-3′ (pegRNA);
(ii) 5′-[spacer]-[crRNA scaffold with U•A flip and tetraloop extension]-[RT template and PBS]-3′ (pegRNA with F+E scaffold);
(iii) 5′-[spacer]-[crRNA scaffold]-[RT template and PBS]-[evopreQ₁ or mpknot]-3′ (epegRNA);
(iv) 5′-[spacer]-[crRNA scaffold]-[RT template and PBS]-[Zika xrRNA]-3′ (xr-pegRNA);
(v) 5′-[spacer]-[crRNA scaffold]-[RT template and PBS]-[G-quadruplex]-3′ (G-PE);
(vi) 5′-[spacer]-[crRNA scaffold with U•A to C•G base pair flip]-[RT template and PBS]-[spacer]-[crRNA scaffold]-3′ (ePE);
(vii) 5′-[spacer]-[crRNA scaffold with C•G-to-G•C pair flip and stabilized stem loop 2]-[RT template and PBS]-3′ (apegRNA); or
(viii) cyclic[MS2 hairpin-RT template and PBS-Rtcb ligation scar]

### Linkers, NLS, and Other PE Elements

### Linkers

In some embodiments, a linker joins a gRNA binding domain of an RNA-programmable nuclease and the catalytic domain of a polymerase (e.g., a reverse transcriptase). In some embodiments, a linker joins a dCas9 and reverse transcriptase. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may included functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises the amino acid sequence (GGGGS)n (SEQ ID NO: 48), (G)n (SEQ ID NO: 49), (EAAAK)n (SEQ ID NO: 50), (GGS)n (SEQ ID NO: 51), (SGGS)n (SEQ ID NO: 52), (XP)n (SEQ ID NO: 53), or any combination thereof, wherein n is independently an integer between 1 and 30, and wherein X is any amino acid. In some embodiments, the linker comprises the amino acid sequence (GGS)n (SEQ ID NO: 54), wherein n is 1, 3, or 7. In some embodiments, the linker comprises the amino acid sequence SGSETPGTSESATPES (SEQ ID NO: 62). In some embodiments, the linker comprises the amino acid sequence SGGSSGGSSGSETPGTSESATPESSGGSSGGS (SEQ ID NO: 56). In some embodiments, the linker comprises the amino acid sequence SGGSGGSGGS (SEQ ID NO: 57). In some embodiments, the linker comprises the amino acid sequence SGGS (SEQ ID NO: 58). In other embodiments, the linker comprises the amino acid sequence SGGSSGGSSGSETPGTSESATPESAGSYPYDVPDYAGSAAPAAKKKKLDGSGSGGSS GGS (SEQ ID NO: 63). Other sequences are also possible.

In particular, the following linkers can be used in various embodiments to join prime editor domains with one another:

(SEQ ID NO: 59)
GGS;
(SEQ ID NO: 60)
GGSGGS;
(SEQ ID NO: 61)
GGSGGSGGS;
(SEQ ID NO: 23)
SGGSSGGSSGSETPGTSESATPESSGGSSGGSS;
(SEQ ID NO: 62)
SGSETPGTSESATPES;
(SEQ ID NO: 63)
SGGSSGGSSGSETPGTSESATPESAGSYPYDVP
DYAGSAAPAAKKKKLDGSGSGGSSGG S

The PE fusion proteins may also comprise various other domains besides the napDNAbp (e.g., Cas9 domain) and the polymerase domain (e.g., RT domain). For example, in the case where the napDNAbp is a Cas9 and the polymerase is a RT, the PE fusion proteins may comprise one or more linkers that join the Cas9 domain with the RT domain. The linkers may also join other functional domains, such as nuclear localization sequences (NLS) or a FEN1 (or other flap endonuclease) to the PE fusion proteins or a domain thereof, or a recombinase (e.g., an integrase).

### Nuclear Localization Sequences (NLS)

In various embodiments, the PE fusion proteins may comprise one or more nuclear localization sequences (NLS), which help promote translocation of a protein into the cell nucleus. Such sequences are well-known in the art and thus, are not provided herein.

The PE fusion proteins may comprise any known NLS sequence, including any of those described in Cokol et al., “Finding nuclear localization signals,” EMBO Rep., 2000, 1(5): 411-415 and Freitas et al., “Mechanisms and Signals for the Nuclear Import of Proteins,” Current Genomics, 2009, 10(8): 550-7, Plank et al., International PCT application PCT/EP2000/011690, filed Nov. 23, 2000, published as WO/2001/038547 on May 31, 2001, the contents of which are incorporated herein by reference.

In various embodiments, the prime editors and constructs encoding the prime editors utilized in the methods and compositions disclosed herein further comprise one or more, preferably, at least two nuclear localization signals. In certain embodiments, the prime editors comprise at least two NLSs. In embodiments with at least two NLSs, the NLSs can be the same NLSs or they can be different NLSs. In addition, the NLSs may be expressed as part of a fusion protein with the remaining portions of the prime editors. In some embodiments, one or more of the NLSs are bipartite NLSs (“bpNLS”). In certain embodiments, the disclosed fusion proteins comprise two bipartite NLSs. In some embodiments, the disclosed fusion proteins comprise more than two bipartite NLSs.

The location of the NLS fusion can be at the N-terminus, the C-terminus, or within a sequence of a prime editor (e.g., inserted between the encoded napDNAbp component (e.g., Cas9) and a polymerase domain (e.g., a reverse transcriptase domain).

The NLSs may be any known NLS sequence in the art. The NLSs may also be any future-discovered NLSs for nuclear localization. The NLSs also may be any naturally-occurring NLS, or any non-naturally occurring NLS (e.g., an NLS with one or more desired mutations).

Most NLSs can be classified in three general groups: (i) a monopartite NLS exemplified by the SV40 large T antigen NLS (PKKKRKV (SEQ ID NO: 64)): (ii) a bipartite motif consisting of two basic domains separated by a variable number of spacer amino acids and exemplified by the Xenopus nucleoplasmin NLS (KRXXXXXXXXXXKKKL (SEQ ID NO: 65)); and (iii) noncanonical sequences such as M9 of the hnRNP A1 protein, the influenza virus nucleoprotein NLS, and the yeast Gal4 protein NLS (Dingwall and Laskey 1991).

The present disclosure contemplates any suitable means by which to modify a prime editor to include one or more NLSs. In one aspect, the prime editors may be engineered to express a prime editor protein that is translationally fused at its N-terminus or its C-terminus (or both) to one or more NLSs, i.e., to form a prime editor-NLS fusion construct. In other embodiments, the prime editor-encoding nucleotide sequence may be genetically modified to incorporate a reading frame that encodes one or more NLSs in an internal region of the encoded prime editor. In addition, the NLSs may include various amino acid linkers or spacer regions encoded between the prime editor and the N-terminally, C-terminally, or internally-attached NLS amino acid sequence, e.g., and in the central region of proteins. Thus, the present disclosure also provides for nucleotide constructs, vectors, and host cells for expressing fusion proteins that comprise a prime editor and one or more NLSs.

The prime editors utilized in the methods and compositions described herein may also comprise nuclear localization signals which are linked to a prime editor through one or more linkers, e.g., and polymeric, amino acid, nucleic acid, polysaccharide, chemical, or nucleic acid linker element. The linkers within the contemplated scope of the disclosure are not intended to have any limitations and can be any suitable type of molecule (e.g., polymer, amino acid, polysaccharide, nucleic acid, lipid, or any synthetic chemical linker domain) and be joined to the prime editor by any suitable strategy that effectuates forming a bond (e.g., covalent linkage, hydrogen bonding) between the prime editor and the one or more NLSs.

### Flap Endonucleases

In various embodiments, the PE fusion proteins may comprise one or more flap endonucleases (e.g., FEN1), which refers to an enzyme that catalyzes the removal of 5′ single strand DNA flaps. These are naturally occurring enzymes that process the removal of 5′ flaps formed during cellular processes, including DNA replication. The prime editing utilized in the methods and compositions described herein may utilize endogenously supplied flap endonucleases or those provided in trans to remove the 5′ flap of endogenous DNA formed at the target site during prime editing. Flap endonucleases are known in the art and can be found described in Patel et al., “Flap endonucleases pass 5′-flaps through a flexible arch using a disorder-thread-order mechanism to confer specificity for free 5′-ends,” Nucleic Acids Research, 2012, 40(10): 4507-4519 and Tsutakawa et al., “Human flap endonuclease structures, DNA double-base flipping, and a unified understanding of the FEN1 superfamily,” Cell, 2011, 145(2): 198-211 (each of which are incorporated herein by reference).

Other endonucleases that may be utilized by the instant methods to facilitate removal of the 5′ end single strand DNA flap include, but are not limited to (1) trex 2, (2) exol endonuclease (e.g., Keijzers et al., Biosci Rep. 2015, 35(3): e00206)

### Additional PE Elements

In certain embodiments, the prime editors utilized in the methods and compositions described herein may comprise an inhibitor of base repair. The term “inhibitor of base repair” or “IBR” refers to a protein that is capable in inhibiting the activity of a nucleic acid repair enzyme, for example a base excision repair enzyme. In some embodiments, the IBR is an inhibitor of OGG base excision repair. In some embodiments, the IBR is an inhibitor of base excision repair (“iBER”). Exemplary inhibitors of base excision repair include inhibitors of APE1, Endo III, Endo IV. Endo V, Endo VIII, Fpg, hOGG1, hNEIL1, T7 Endol, T4PDG, UDG, hSMUG1, and hAAG. In some embodiments, the IBR is an inhibitor of Endo V or hAAG. In some embodiments, the IBR is an iBER that may be a catalytically inactive glycosylase or catalytically inactive dioxygenase or a small molecule or peptide inhibitor of an oxidase, or variants thereof. In some embodiments, the IBR is an iBER that may be a TDG inhibitor, MBD4 inhibitor or an inhibitor of an AlkBH enzyme. In some embodiments, the IBR is an iBER that comprises a catalytically inactive TDG or catalytically inactive MBD4.

In some embodiments, the fusion proteins described herein may comprise one or more heterologous protein domains (e.g., about or more than about 1, 2, 3, 4, 5, 6, 7, 8, 9, 10, or more domains in addition to the prime editor components). A fusion protein may comprise any additional protein sequence, and optionally a linker sequence between any two domains. Other exemplary features that may be present are localization sequences, such as cytoplasmic localization sequences, export sequences, such as nuclear export sequences, or other localization sequences, as well as sequence tags that are useful for solubilization, purification, or detection of the fusion proteins.

Examples of protein domains that may be fused to a prime editor or component thereof (e.g., the napDNAbp domain, the polymerase domain, or the NLS domain) include, without limitation, epitope tags, and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (CST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and auto fluorescent proteins including blue fluorescent protein (BFP). A prime editor may be fused to a gene sequence encoding a protein or a fragment of a protein that bind DNA molecules or bind other cellular molecules, including, but not limited to, maltose binding protein (MBP), S-tag, Lex A DNA binding domain (DBD) fusions, GAL4 DNA binding domain fusions, and herpes simplex virus (HSV) BP16 protein fusions. Additional domains that may form part of a prime editor are described in US Patent Publication No. 2011/0059502, published Mar. 10, 2011 and incorporated herein by reference in its entirety.

In an aspect of the disclosure, a reporter gene which includes, but is not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto fluorescent proteins including blue fluorescent protein (BFP), may be introduced into a cell to encode a gene product which serves as a marker by which to measure the alteration or modification of expression of the gene product. In certain embodiments of the disclosure the gene product is luciferase. In a further embodiment of the disclosure the expression of the gene product is decreased.

Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

In some embodiments of the present disclosure, the activity of the prime editing system may be temporally regulated by adjusting the residence time, the amount, and/or the activity of the expressed components of the PE system. For example, as described herein, the PE may be fused with a protein domain that is capable of modifying the intracellular half-life of the PE. In certain embodiments involving two or more vectors (e.g., a vector system in which the components described herein are encoded on two or more separate vectors), the activity of the PE system may be temporally regulated by controlling the timing in which the vectors are delivered. For example, in some embodiments a vector encoding the nuclease system may deliver the PE prior to the vector encoding the template. In other embodiments, the vector encoding the pegRNA may deliver the guide prior to the vector encoding the PE system. In some embodiments, the vectors encoding the PE system and pegRNA are delivered simultaneously. In certain embodiments, the simultaneously delivered vectors temporally deliver, e.g., the PE, pegRNA, and/or second strand guide RNA components. In further embodiments, the RNA (such as, e.g., the nuclease transcript) transcribed from the coding sequence on the vectors may further comprise at least one element that is capable of modifying the intracellular half-life of the RNA and/or modulating translational control. In some embodiments, the half-life of the RNA may be increased. In some embodiments, the half-life of the RNA may be decreased. In some embodiments, the element may be capable of increasing the stability of the RNA. In some embodiments, the element may be capable of decreasing the stability of the RNA. In some embodiments, the element may be within the 3′ UTR of the RNA. In some embodiments, the element may include a polyadenylation signal (PA). In some embodiments, the element may include a cap, e.g., an upstream mRNA or pegRNA end. In some embodiments, the RNA may comprise no PA such that it is subject to quicker degradation in the cell after transcription. In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments the destabilizing element may promote RNA decay, affect RNA stability, or activate translation. In some embodiments, the ARE may comprise 50 to 150 nucleotides in length. In some embodiments, the ARE may comprise at least one copy of the sequence AUUUA. In some embodiments, at least one ARE may be added to the 3′ UTR of the RNA. In some embodiments, the element may be a Woodchuck Hepatitis Virus (WHP).

Posttranscriptional Regulatory Element (WPRE), which creates a tertiary structure to enhance expression from the transcript. In further embodiments, the element is a modified and/or truncated WPRE sequence that is capable of enhancing expression from the transcript, as described, for example in Zufferey et al., J Virol, 73(4): 2886-92 (1999) and Flajolet et al., J Virol, 72(7): 6175-80 (1998). In some embodiments, the WPRE or equivalent may be added to the 3′ UTR of the RNA. In some embodiments, the element may be selected from other RNA sequence motifs that are enriched in either fast- or slow-decaying transcripts.

In some embodiments, the vector encoding the PE or the pegRNA may be self-destroyed via cleavage of a target sequence present on the vector by the PE system. The cleavage may prevent continued transcription of a PE or a pegRNA from the vector. Although transcription may occur on the linearized vector for some amount of time, the expressed transcripts or proteins subject to intracellular degradation will have less time to produce off-target effects without continued supply from expression of the encoding vectors.

### Kits, Cells, and Vectors

### Kits

The compositions of the present disclosure may be assembled into kits. In some embodiments, the kit comprises nucleic acid vectors for the expression of a prime editor. In other embodiments, the kit further comprises appropriate guide nucleotide sequences (e.g., pegRNAs and second-site gRNAs) or nucleic acid vectors for the expression of such guide nucleotide sequences, to target the Cas9 protein or prime editor to the desired target sequence.

The kit described herein may include one or more containers housing components for performing the methods described herein and optionally instructions for use. Any of the kits described herein may further comprise components needed for performing the assay methods. Each component of the kits, where applicable, may be provided in liquid form (e.g., in solution) or in solid form, (e.g., a dry powder). In certain cases, some of the components may be reconstitutable or otherwise processible (e.g., to an active form), for example, by the addition of a suitable solvent or other species (for example, water), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use, or sale of pharmaceuticals or biological products, which can also reflect approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the disclosure. Additionally, the kits may include other components depending on the specific application, as described herein.

The kits may contain any one or more of the components described herein in one or more containers. The components may be prepared sterilely, packaged in a syringe and shipped refrigerated. Alternatively it may be housed in a vial or other container for storage. A second container may have other components prepared sterilely. Alternatively the kits may include the active agents premixed and shipped in a vial, tube, or other container.

The kits may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc. Some aspects of this disclosure provide kits comprising a nucleic acid construct comprising a nucleotide sequence encoding the various components of the prime editing system utilized in the methods and compositions described herein (e.g., including, but not limited to, the napDNAbps, reverse transcriptases, polymerases, fusion proteins (e.g., comprising napDNAbps and reverse transcriptases (or more broadly, polymerases), extended guide RNAs, and complexes comprising fusion proteins and extended guide RNAs, as well as accessory elements, such as second strand nicking components (e.g., second strand nicking gRNA) and 5′ endogenous DNA flap removal endonucleases for helping to drive the prime editing process towards the edited product formation). In some embodiments, the nucleotide sequence(s) comprises a heterologous promoter (or more than a single promoter) that drives expression of the prime editing system components.

Other aspects of this disclosure provide kits comprising one or more nucleic acid constructs encoding the various components of the prime editing systems utilized in the methods and compositions described herein, e.g., the comprising a nucleotide sequence encoding the components of the prime editing system capable of modifying a target DNA sequence. In some embodiments, the nucleotide sequence comprises a heterologous promoter that drives expression of the prime editing system components.

Some aspects of this disclosure provides kits comprising a nucleic acid construct, comprising (a) a nucleotide sequence encoding a napDNAbp (e.g., a Cas9 domain) fused to a reverse transcriptase and (b) a heterologous promoter that drives expression of the sequence of (a).

### Cells

Cells that may contain any of the compositions described herein include prokaryotic cells and eukaryotic cells. The methods described herein are used to deliver a prime editor and a pegRNA into a eukaryotic cell (e.g., a mammalian cell, such as a human cell). In some embodiments, the cell is in vitro (e.g., cultured cell). In some embodiments, the cell is in vivo (e.g., in a subject such as a human subject). In some embodiments, the cell is ex vivo (e.g., isolated from a subject and may be administered back to the same or a different subject).

Mammalian cells of the present disclosure include human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NC160), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells. THP-1 (acute myeloid leukemia) cells, U87 (glioblastoma) cells, SHSY5Y human neuroblastoma cells (cloned from a myeloma) and Saos-2 (bone cancer) cells. In some embodiments, rAAV vectors are delivered into human embryonic kidney (HEK) cells (e.g., HEK 293 or HEK 293T cells). In some embodiments, rAAV vectors are delivered into stem cells (e.g., human stem cells) such as, for example, pluripotent stem cells (e.g., human pluripotent stem cells including human induced pluripotent stem cells (hiPSCs)). A stem cell refers to a cell with the ability to divide for indefinite periods in culture and to give rise to specialized cells. A pluripotent stem cell refers to a type of stem cell that is capable of differentiating into all tissues of an organism, but not alone capable of sustaining full organismal development. A human induced pluripotent stem cell refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cell cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepa1c1c7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRC5, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39. WT-49, X63, YAC-1 and YAR cells.

Some aspects of this disclosure provide cells comprising any of the constructs disclosed herein. In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620. SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-IR, CHO-K1, CHO-K2, CHO-T, CHO Dhfr −/−, COR-L23. COR-L23/CPR, COR-L23/5010. COR-L23/R23, COS-7, COV-434, CML T1, CMT, CT26, D17, DH82, D1U145, DuCaP, EL4, EM2, EM3, EMT6/AR1, EMT6/AR10.0, FM3, H1299. H69, HB54, HB55, HCA2, HEK-293, HeLa, Hepa1c1c7, HL-60, HMEC, HT-29. Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof.

Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

### Vectors

Some aspects of the present disclosure relate to using recombinant virus vectors (e.g., adeno-associated virus vectors, adenovirus vectors, or herpes simplex virus vectors) for the delivery of the prime editors and pegRNAs as described herein into a cell. In the case of a split-PE approach, the N-terminal portion of a PE fusion protein and the C-terminal portion of a PE fusion are delivered by separate recombinant virus vectors (e.g., adeno-associated virus vectors, adenovirus vectors, or herpes simplex virus vectors) into the same cell, since the full-length Cas9 protein or prime editors exceeds the packaging limit of various virus vectors, e.g., rAAV (˜4.9 kb).

In some embodiments, the vectors used herein may encode the PE fusion proteins, or any of the components thereof (e.g., napDNAbp, linkers, or polymerases). In addition, the vectors used herein may encode the pegRNAs, and/or the accessory gRNA for second strand nicking. The vectors may be capable of driving expression of one or more coding sequences in a cell. In some embodiments, the cell may be a prokaryotic cell, such as, e.g., a bacterial cell. In some embodiments, the cell may be a eukaryotic cell, such as, e.g., a yeast, plant, insect, or mammalian cell. In some embodiments, the eukaryotic cell may be a mammalian cell. In some embodiments, the eukaryotic cell may be a rodent cell. In some embodiments, the eukaryotic cell may be a human cell. Suitable promoters to drive expression in different types of cells are known in the art. In some embodiments, the promoter may be wild-type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the promoters that may be used in the prime editor vectors may be constitutive, inducible, or tissue-specific. In some embodiments, the promoters may be a constitutive promoters. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EFla) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EFla promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech). In some embodiments, the promoter may be a tissue-specific promoter. In some embodiments, the tissue-specific promoter is exclusively or predominantly expressed in liver tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, Nphs1 promoter, OG-2 promoter, SP-B promoter, SYN1 promoter, and WASP promoter.

In some embodiments, the prime editor pegRNA vectors (e.g., including any vectors encoding the prime editor fusion protein and/or the pegRNAs, and/or the accessory second strand nicking gRNAs) may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In additional embodiments, the prime editor vectors (e.g., including any vectors encoding the prime editor fusion protein and/or the pegRNAs, and/or the accessory second strand nicking gRNAs) may comprise tissue-specific promoters to start expression only after it is delivered into a specific tissue. Non-limiting exemplary tissue-specific promoters include B29 promoter, CDI 4 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, Nphs1 promoter, OG-2 promoter, SP-B promoter, SYN1 promoter, and WASP promoter.

In some embodiments, the nucleotide sequence encoding the pegRNA (or any guide RNAs used in connection with prime editing) may be operably linked to at least one transcriptional or translational control sequence. In some embodiments, the nucleotide sequence encoding the guide RNA may be operably linked to at least one promoter. In some embodiments, the promoter may be recognized by RNA polymerase III (Pol III). Non-limiting examples of Pol III promoters include U6, HI and tRNA promoters. In some embodiments, the nucleotide sequence encoding the guide RNA may be operably linked to a mouse or human U6 promoter. In other embodiments, the nucleotide sequence encoding the guide RNA may be operably linked to a mouse or human HI promoter. In some embodiments, the nucleotide sequence encoding the guide RNA may be operably linked to a mouse or human tRNA promoter. In embodiments with more than one guide RNA, the promoters used to drive expression may be the same or different. In some embodiments, the nucleotide encoding the crRNA of the guide RNA and the nucleotide encoding the tracr RNA of the guide RNA may be provided on the same vector. In some embodiments, the nucleotide encoding the crRNA and the nucleotide encoding the tracr RNA may be driven by the same promoter. In some embodiments, the crRNA and tracr RNA may be transcribed into a single transcript. For example, the crRNA and tracr RNA may be processed from the single transcript to form a double-molecule guide RNA. Alternatively, the crRNA and tracr RNA may be transcribed into a single-molecule guide RNA.

In some embodiments, the nucleotide sequence encoding the guide RNA may be located on the same vector comprising the nucleotide sequence encoding the PE fusion protein. In some embodiments, expression of the guide RNA and of the PE fusion protein may be driven by their corresponding promoters. In some embodiments, expression of the guide RNA may be driven by the same promoter that drives expression of the PE fusion protein. In some embodiments, the guide RNA and the PE fusion protein transcript may be contained within a single transcript. For example, the guide RNA may be within an untranslated region (UTR) of the Cas9 protein transcript. In some embodiments, the guide RNA may be within the 5′ UTR of the PE fusion protein transcript. In other embodiments, the guide RNA may be within the 3′ UTR of the PE fusion protein transcript. In some embodiments, the intracellular half-life of the PE fusion protein transcript may be reduced by containing the guide RNA within its 3′ UTR and thereby shortening the length of its 3′ UTR. In additional embodiments, the guide RNA may be within an intron of the PE fusion protein transcript. In some embodiments, suitable splice sites may be added at the intron within which the guide RNA is located such that the guide RNA is properly spliced out of the transcript. In some embodiments, expression of the Cas9 protein and the guide RNA in close proximity on the same vector may facilitate more efficient formation of the CRISPR complex.

The vector system may comprise one vector, or two vectors, or three vectors, or four vectors, or five vector, or more. In some embodiments, the vector system may comprise one single vector, which encodes both the PE fusion protein and the pegRNA. In other embodiments, the vector system may comprise two vectors, wherein one vector encodes the PE fusion protein, and the other encodes the pegRNA.

### Virus-Lipid Particle (VLPs)

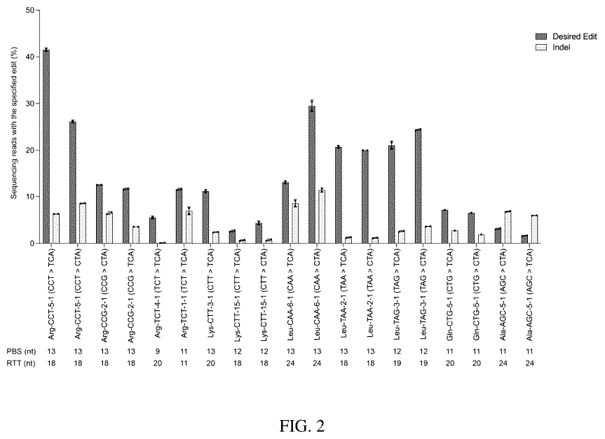

Aspects of the disclosure relate to delivering a prime editing system using VLPs, such as those described in U.S. Patent Application Ser. No. 63/298,626 filed on Jan. 11, 2022, which is herein incorporated by reference in its entirety. In various embodiments, the eVLPs (e.g., PE-VLPs) consist of a supra-molecular assembly comprising (a) an envelope comprising (i) a lipid membrane (e.g., single-layer or bi-layer membrane) and a (ii) viral envelope glycoprotein (e.g., VSV-G) and (b) a multi-protein core region enclosed by the envelope and comprising (i) a Gag protein, (ii) a Gag-Pro-Pol protein (with the “Pro” component referring to a protease), and (iii) a Gag-cargo fusion protein comprising a Gag protein fused to a cargo protein (e.g., a napDNAbp or PE or a split PE) via a cleavable linker (e.g., a protease-cleavable linker). In various embodiments, the cargo protein is a napDNAbp (e.g., Cas9). In other embodiments, the cargo protein is a prime editor. In various embodiments (e.g., FIG. 2A) the PE may be split into a Cas9 domain and a reverse transcriptase domain as separate fusion proteins each with Gag. In various embodiments, the split domains of PE may comprise split-intein sequences which allows the split domains to re-form a PE once delivered to a cell. In various other embodiments, the multi-protein core region of the VLPs further comprises one or more pegRNA molecules and/or second-site nicking guide RNA which are complexed with the napDNAbp or the prime editor to form a ribonucleoprotein (RNP).

In various embodiments, the VLPs are prepared in a producer cell that is transiently transformed with plasmid DNA that encodes the various protein and nucleic acid (pegRNAs and guide RNAs) components of the VLPs. Without being bound by theory, the components self-assemble at the cell membrane and bud out in accordance with the naturally occurring mechanism of budding (e.g., retroviral budding or the budding mechanism of other envelope viruses) in order to release from the cell fully-matured VLPs. Once formed, the Gag-Pol-Pro cleaves the protease-sensitive linker of the Gag-cargo (i.e., [Gag]-[cleavable linker]-[cargo], wherein the cargo can be PE-RNP or a napDNAbp RNP) thereby releasing the PE RNP and/or napDNAbp RNA, as the case may be, within the VLP. Once the VLP is administered to a recipient cell and taken up by said recipient cell, the contents of the VLP are released, e.g., released PE RNP and/or napDNAbp RNP. Once in the cell, the RNPs may translocate to the nuclease of the cell (in particular, where NLSs are included on the RNPs), where DNA editing may occur at target sites specified by the guide RNA. Various embodiments comprise one or more improvements.

In one improvement, the protease-cleavable linker is optimized to improve cleavage efficiency after VLP maturation, as demonstrated herein for v.2 VLPs (or “second generation” VLPs).

In another improvement, the Gag-cargo fusion (e.g., Gag-BE) further comprises one or more nuclear export signals at one or more locations along the length of the fusion polypeptide protein which may be joined by a cleavable linker such that during VLP assembly in the producer cell, the Gag-cargo fusions (due to presence of competing NLS signals) do not accumulate in the nucleus of the producer cells but instead are available in the cytoplasm to undergo the VLP assembly process at the cell membrane. Once inside the matured VLPs following release from the producer cell, the NES may be cleaved by Gag-Pro-Pol thereby separating the cargo (e.g., napDNAbp or a PE) from the NES. Upon delivery to a recipient cell, therefore, the cargo (e.g., napDNAbp or PE, typically flanked with one or more NLS elements) will not comprise an NES element, which may otherwise prohibit the transport of the cargo into the nuclease and hinder gene editing activity. This is exemplified as v.3 VLPs described herein (or “third generation” VLPs).

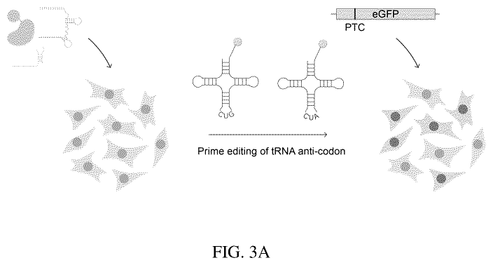

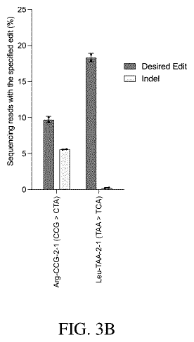

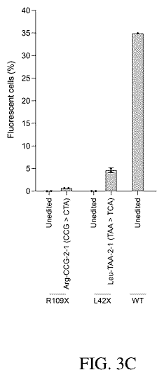

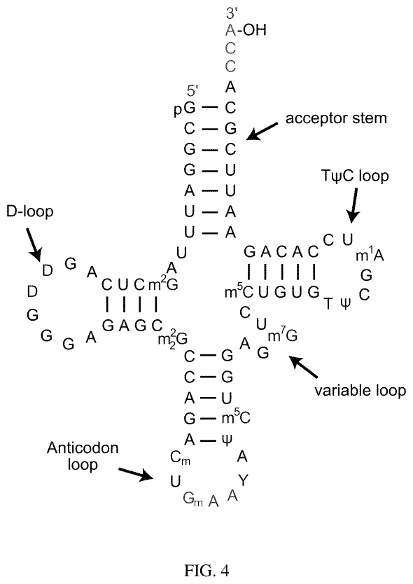

In other embodiments, the eVLPs disclosed herein may comprise split PE domains contained in a single all-in-one VLP system or in a two-particle system whereby each PE half domain is formed in separate VLPs. See FIG. 3A.

In one aspect, the present disclosure provides a eVLP comprising an (a) envelope and (b) a multi-protein core, wherein the envelope comprises a lipid membrane (e.g., a lipid mono or bi-layer membrane) and a viral envelope glycoprotein and wherein the multi-protein core comprises a Gag (e.g., a retroviral Gag), a group-specific antigen (gag) protease (pro) polyprotein (i.e., “Gag-Pro-Pol”) and a fusion protein comprising a Gag-cargo (e.g., Gag-napDNAbp or Gag-PE). In various embodiments, the Gag-cargo may comprise a ribonucleoprotein cargo, e.g., a napDNAbp or a PE complexed with a guide RNA. In still further embodiments, the Gag-cargo (e.g., Gag fused to a napDNAbp or a PE) may comprise one or more NLS sequences and/or one or more NES sequences to regulate the cellular location of the cargo in a cell. An NLS sequence will facilitate the transport of the cargo into the cell's nuclease to facilitate editing. A NES will do the opposite, i.e., transport the cargo out from the nucleus, and/or prevent the transport of the cargo into the nucleus. In certain embodiments, the NES may be coupled to the fusion protein by a cleavable linker (e.g., a protease linker) such that during assembly in a producer cell, the NES signals and operates to keep the cargo in the cytoplasm and available for the packaging process. However, once matured VLPs are budded out or released from a producer cell in a mature form, the cleavable linker joining the NES may be cleaved, thereby removing the association of NES with the cargo. Thus, without an NES, the cargo will translocate to the nuclease with its NLS sequences, thereby facilitating editing. Various napDNAbps may be used in the systems of the present disclosure. In some embodiments, the napDNAbp is a Cas9 protein (e.g., a Cas9 nickase, dead Cas9 (dCas9), or another Cas9 variant as described herein). In some embodiments, the Cas9 protein is bound to a guide RNA (gRNA). The fusion protein may further comprise other protein domains, such as effector domains. In some embodiments, the fusion protein further comprises a deaminase domain (e.g., an adenosine deaminase domain or a cytosine deaminase domain). In certain embodiments, the fusion protein comprises a prime editor, such as PE2, PE3, or PEmax prime editor, or any of the other prime editors described herein or known in the art.

In some embodiments, the fusion protein comprises more than one NES (e.g., two NES, three NES, four NES, five NES, six NES, seven NES, eight NES, nine NES, or ten or more NES). In certain embodiments, the fusion protein further comprises a nuclear localization sequence (NLS), or more than one NLS (e.g., two NLS, three NLS, four NLS, five NLS, six NLS, seven NLS, eight NLS, nine NLS, or ten or more NLS). In certain embodiments, the fusion protein may comprise at least one NES and one NLS.

The Gag-cargo fusion proteins described herein comprise one or more cleavable linkers. In one embodiment, the Gag-cargo fusion proteins comprise a cleavable linker joining the Gag to the cargo, such that once the Gag-cargo fusion has been packaged in mature VLPs (which will also contain the Gag-Pro-Pol, the protease activity can cleave the Gag-cargo cleavable linker, thereby releasing the cargo. In some embodiments, a cleavable linker may also be provided in such a location such that when the cleavable linker is cleaved (e.g., by the Gag-Pro-Pol protein), the NES is separated away from the cargo protein. Such an arrangement of the fusion protein allows the fusion protein to be exported from the nucleus of a producing cell during BE-VLP production, and the NES can later be cleaved from the fusion protein after delivery to a target cell, releasing the PE (or release of split PE half domains from the same or a two-particle system) and allowing it to enter the nucleus of the target cell. In some embodiments, the cleavable linker comprises a protease cleavage site (e.g., a Moloney murine leukemia virus (MMLV) protease cleavage site or a Friend murine leukemia virus (FMLV) protease cleavage site). Various protease cleavage sites can be used in the fusion proteins of the present disclosure. In certain embodiments, the protease cleavage site comprises the amino acid sequence TSTLLMENSS (SEQ ID NO: 66), PRSSLYPALTP (SEQ ID NO: 67), VQALVLTQ (SEQ ID NO: 68), PLQVLTLNIERR (SEQ ID NO: 69), or an amino acid sequence at least 90% identical to any one of SEQ ID NOs: 66-69. In some embodiments, the cleavable linker of the fusion protein is cleaved by the protease of the gag-pro polyprotein. In certain embodiments, the cleavable linker of the fusion protein is not cleaved by the protease of the gag-pro polyprotein until the PE-VLP has been assembled and delivered into a target cell. In some embodiments, the gag-pro polyprotein of the PE-VLPs described herein comprises an MMLV gag-pro polyprotein or an FMLV gag-pro polyprotein. In some embodiments, the gag nucleocapsid protein of the fusion protein in the PE-VLPs described herein comprises an MMLV gag nucleocapsid protein or an FMLV gag nucleocapsid protein.

In certain embodiments, the fusion protein comprises the following non-limiting structures:
[gag nucleocapsid protein]-[1X-3X NES]-[cleavable linker]-[NLS]-[RT domain]-[napDNAbp]-[NLS], wherein]-[comprises an optional linker (e.g., an amino acid linker, or any of the linkers provided herein);
[1X-3X NES]-[gag nucleocapsid protein]-[cleavable linker]-[NLS]-[RT domain]-[napDNAbp]-[NLS], wherein 1-[comprises an optional linker (e.g., an amino acid linker, or any of the linkers provided herein); or
[gag nucleocapsid protein]-[1X-3X NES]-[cleavable linker]-[NLS]-[RT domain]-[napDNAbp]-[NLS]-[cleavable linker]-[1X-3X NES], wherein]-[comprises an optional linker (e.g., an amino acid linker, or any of the linkers provided herein).

The eVLPs (e.g., the PE-VLPs) provided by the present disclosure comprise an outer encapsulation layer (or envelope layer) comprising a viral envelope glycoprotein. Any viral envelope glycoprotein described herein, or known in the art, may be used in the PE-VLPs of the present disclosure. In some embodiments, the viral envelope glycoprotein is an adenoviral envelope glycoprotein, an adeno-associated viral envelope glycoprotein, a retroviral envelope glycoprotein, or a lentiviral envelope glycoprotein. In certain embodiments, the viral envelope glycoprotein is a retroviral envelope glycoprotein. In some embodiments, the viral envelope glycoprotein is a vesicular stomatitis virus G protein (VSV-G), a baboon retroviral envelope glycoprotein (BaEVRless), a FuG-B2 envelope glycoprotein, an HTV-1 envelope glycoprotein, or an ecotropic murine leukemia virus (MLV) envelope glycoprotein. In some embodiments, the viral envelope glycoprotein targets the system to a particular cell type (e.g., immune cells, neural cells, retinal pigment epithelium cells, etc.). For example, using different envelope glycoproteins in the eVLPs described herein may alter their cellular tropism, allowing the PE-VLPs to be targeted to specific cell types. In some embodiments, the viral envelope glycoprotein is a VSV-G protein, and the VSV-G protein targets the system to retinal pigment epithelium (RPE) cells. In some embodiments, the viral envelope glycoprotein is an HIV-1 envelope glycoprotein, and the HIV-1 envelope glycoprotein targets the system to CD4+ cells. In some embodiments, the viral envelope glycoprotein is a FuG-B2 envelope glycoprotein, and the FuG-B2 envelope glycoprotein targets the system to neurons.

It will be appreciated that general methods are known in the art for producing viral vector particles, which generally contain coding nucleic acids of interest, may also be used for producing the virus-derived particles according to the present invention, which do not contain coding nucleic acids of interest but instead are designed to deliver a protein cargo (e.g., a PE RNP).

Conventional viral vector particles encompass retroviral, lentiviral, adenoviral and adeno-associated viral vector particles that are well known in the art. For a review of various viral vector particles that may be used, the one skilled in the art may notably refer to Kushnir et al. (2012, Vaccine, Vol. 31: 58-83), Zeltons (2013, Mol Biotechnol, Vol. 53: 92-107), Ludwig et al. (2007. Curr Opin Biotechnol, Vol. 18(no 6): 537-55) and Naskalaska et al. (2015, Vol. 64 (no 1): 3-13). Further, references to various methods using virus-derived particles for delivering proteins to cells are found by the one skilled in the art in the article of Maetzig et al. (2012, Current Gene therapy, Vol. 12: 389-409) as well as the article of Kaczmarczyk et al. (2011, Proc Natd Acad Sci USA, Vol. 108 (no 41): 16998-17003).

Generally, a virus-like particle that is used according to the present disclosure, which virus-like particle may also be termed “virus-derived particle,” is formed by one or more virus-derived structural protein(s) and/or one more virus-derived envelope protein.

A virus-like particle that is used according to the present invention is replication incompetent in a host cell wherein it has entered.

In preferred embodiments, a virus-like particle is formed by one or more retrovirus-derived stuctural protein(s) and optionally one or more virus-derived envelope protein(s).

In preferred embodiments, the virus-derived structural protein is a retroviral Gag protein or a peptide fragment thereof. As it is known in the art, Gag and Gag/pol precursors are expressed from full length genomic RNA as polyproteins, which require proteolytic cleavage, mediated by the retroviral protease (PR), to acquire a functional conformation. Further, Gag, which is structurally conserved among the retroviruses, is composed of at least three protein units: matrix protein (MA), capsid protein (CA) and nucleocapsid protein (NC), whereas Pol consists of the retroviral protease, (PR), the retrotranscriptase (RT) and the integrase (IN).

In some embodiments, a virus-derived particle comprises a retroviral Gag protein but does not comprise a Pol protein.

As it is known in the art, the host range of retroviral vector, including lentiviral vectors, may be expanded or altered by a process known as pseudotyping. Pseudotyped lentiviral vectors consist of viral vector particles bearing glycoproteins derived from other enveloped viruses. Such pseudotyped viral vector particles possess the tropism of the virus from which the glycoprotein is derived.

In some embodiments, a virus-like particle is a pseudotyped virus-like particle comprising one or more viral structural protein(s) or viral envelope protein(s) imparting a tropism to the said virus-like particle for certain eukaryotic cells. A pseudotyped virus-like particle as described herein may comprise, as the viral protein used for pseudotyping, a viral envelope protein selected in a group comprising VSV-G protein, Measles virus HA protein, Measles virus F protein, Influenza virus HA protein, Moloney virus MLV-A protein, Moloney virus MLV-E protein, Baboon Endogenous retrovirus (BAEV) envelope protein, Ebola virus glycoprotein and foamy virus envelope protein, or a combination of two or more of these viral envelope proteins.

A well-known illustration of pseudotyping viral vector particles consists of the pseudotyping of viral vector particles with the vesicular stomatitis virus glycoprotein (VSV-G). For the pseudotyping of viral vector particles, the one skilled in the art may notably refer to Yee et al. (1994, Proc Natl Acad Sci, USA, Vol. 91: 9564-9568) Cronin et al. (2005, Curr Gene Ther, Vol. 5(no 4): 387-398), which are incorporated herein by reference.

For producing virus-like particles, and more precisely VSV-G pseudotypes virus-like particles, for delivering protein(s) of interest into target cells, the one skilled in the art may refer to Mangeot et al. (2011, Molecular Therapy, Vol. 19 (no 9): 1656-1666).

In some embodiments, a virus-like particle further comprises a viral envelope protein, wherein either (i) the said viral envelope protein originates from the same virus as the viral structural protein, e.g., originates from the same virus as the viral Gag protein, or (ii) the said viral envelope protein originates from a virus distinct from the virus from which originates the viral structural protein, e.g. originates from a virus distinct from the virus from which originates the viral Gag protein.

As it is readily understood by the one skilled in the art, a virus-like particle that is used according to the disclosure may be selected in a group comprising Moloney murine leukemia virus-derived vector particles, Bovine immunodeficiency virus-derived particles, Simian immunodeficiency virus-derived vector particles, Feline immunodeficiency virus-derived vector particles, Human immunodeficiency virus-derived vector particles, Equine infection anemia virus-derived vector particles, Caprine arthritis encephalitis virus-derived vector particle, Baboon endogenous virus-derived vector particles, Rabies virus-derived vector particles, Influenza virus-derived vector particles, Norovirus-derived vector particles, Respiratory syncytial virus-derived vector particles. Hepatitis A virus-derived vector particles, Hepatitis B virus-derived vector panicles, Hepatitis E virus-derived vector particles, Newcastle disease virus-derived vector particles, Norwalk virus-derived vector particles, Parvovirus-derived vector particles, Papillomavirus-derived vector particles, Yeast retrotransposon-derived vector panicles. Measles virus-derived vector particles, and bacteriophage-derived vector particles.

In particular, a virus-like particle that is used according to the invention is a retrovirus-derived particle. Such retrovirus may be selected among Moloney murine leukemia virus, Bovine immunodeficiency virus, Simian immunodeficiency virus, Feline immunodeficiency virus, Human immunodeficiency virus, Equine infection anemia virus, and Caprine arthritis encephalitis virus.

In another embodiment, a virus-like particle that is used according to the disclosure is a lentivirus-derived particle. Lentiviruses belong to the retroviruses family, and have the unique ability of being able to infect non-dividing cells.

Such lentivirus may be selected among Bovine immunodeficiency virus, Simian immunodeficiency virus, Feline immunodeficiency virus, Human immunodeficiency virus, Equine infection anemia virus, and Caprine arthritis encephalitis virus.

For preparing Moloney murine leukemia virus-derived vector particles, one skilled in the art may refer to the methods disclosed by Sharma et al. (1997, Proc Natl Acad Sci USA, Vol. 94: 10803+-10808), Guibingua et al. (2002, Molecular Therapy, Vol. 5(no 5): 538-546), which are incorporated herein by reference. Moloney murine leukemia virus-derived (MLV-derived) vector particles may be selected in a group comprising MLV-A-derived vector panicles and MLV-E-derived vector particles.

For preparing Bovine Immunodeficiency virus-derived vector panicles, the one skilled in the art may refer to the methods disclosed by Rasmussen et al. (1990, Virology, Vol. 178(no 2): 435-451), which is incorporated herein by reference.

For preparing Simian immunodeficiency virus-derived vector particles, including VSV-G pseudotyped SIV virus-derived particles, the one skilled in the art may notably refer to the methods disclosed by Mangeot et al. (2000, Journal of Virology, Vol. 71(no 18): 8307-8315), Negre et al. (2000, Gene Therapy, Vol. 7: 1613-1623) Mangeot et al. (2004, Nucleic Acids Research, Vol. 32 (no 12), e102), which are incorporated herein by reference.

For preparing Feline Immunodeficiency virus-derived vector particles, the one skilled in the art may notably refer to the methods disclosed by Saenz et al. (2012, Cold Spring Harb Protoc, (1): 71-76; 2012. Cold Spring Harb Protoc, (1): 124-125; 2012, Cold Spring Harb Protoc, (1): 118-123), which are incorporated herein by reference.

For preparing Human immunodeficiency virus-derived vector particles, the one skilled in the art may notably refer to the methods disclosed by Jalaguier et al. (2011, PlosOne, Vol. 6(no 11), e28314), Cervera et al. (J Biotechnol, Vol. 166(no 4): 152-165), Tang et al. (2012, Journal of Virology, Vol. 86(no 14): 7662-7676), which are incorporated herein by reference.

For preparing Equine infection anemia virus-derived vector particles, the one skilled in the art may notably refer to the methods disclosed by Olsen (1998, Gene Ther, Vol. 5(no 11): 1481-1487), which are incorporated herein by reference.

For preparing Caprine arthritis encephalitis virus-derived vector particles, the one skilled in the art may notably refer to the methods disclosed by Mselli-Lakhal et al. (2006, J Virol Methods, Vol. 136(no 1-2): 177-184), which are incorporated herein by reference.

For preparing Baboon endogenous virus-derived vector particles, the one skilled in the art may notably refer to the methods disclosed by Girard-Gagnepain et al. (2014, Blood, Vol. 124(no 8): 1221-1231), which is incorporated herein by reference.

For preparing Rabies virus-derived vector particles, the one skilled in the art may notably refer to the methods disclosed by Kang et al. (2015, Viruses, Vol. 7: 1134-1152, doi:10.3390/v7031134), Fontana et al. (2014, Vaccine, Vol. 32(no 24): 2799-27804) or to the PCT application published under no WO 2012/0618, which is incorporated herein by reference.

For preparing Influenza virus-derived vector particles, the one skilled in the art may notably refer to the methods disclosed by Quan et al. (2012, Virology, Vol. 430: 127-135) and to Latham et al. (2001, Journal of Virology, Vol. 75(no 13): 6154-6155), which is incorporated herein by reference.

For preparing Norovirus-derived vector particles, the one skilled in the art may notably refer to the methods disclosed by Tomd-Amat et al., (2014, Microbial Cell Factories, Vol. 13: 134-142), which is incorporated herein by reference.

For preparing Respiratory syncytial virus-derived vector particles, the one skilled in the art may notably refer to the methods disclosed by Walpita et al. (2015, PlosOne, DOI: 10.1371/journal.pone.0130755), which is incorporated herein by reference.

For preparing Hepatitis B virus-derived vector particles, the one skilled in the art may notably refer to the methods disclosed by Hong et al. (2013, Vol. 87(no 12): 6615-6624), which is incorporated herein by reference.

For preparing Hepatitis E virus-derived vector particles, the one skilled in the art may notably refer to the methods disclosed by Li et al. (1997, Journal of Virology, Vol. 71(no 10): 7207-7213), which is incorporated herein by reference.

For preparing Newcastle disease virus-derived vector particles, the one skilled in the art may notably refer to the methods disclosed by Murawski et al. (2010, Journal of Virology, Vol. 84(no 2): 1110-1123), which is incorporated herein by reference.

For preparing Norwalk virus-derived vector particles, the one skilled in the art may notably refer to the methods disclosed by Herbst-Kralovetz et al. (2010, Expert Rev Vaccines, Vol. 9(no 3): 299-307), which is incorporated herein by reference.

For preparing Parvovirus-derived vector particles, the one skilled in the art may notably refer to the methods disclosed by Ogasawara et al. (2006, In Vivo, Vol. 20: 319-324), which is incorporated herein by reference.

For preparing Papillomavirus-derived vector particles, the one skilled in the art may notably refer to the methods disclosed by Wang et al. (2013, Expert Rev Vaccines, Vol. 12(no 2): doi:10.1586/erv.12.151), which is incorporated herein by reference.

A virus-like particle that is used herein comprises a Gag protein, and most preferably a Gag protein originating from a virus selected in a group comprising Rous Sarcoma Virus (RSV) Feline Immunodeficiency Virus (FIV), Simian Immunodeficiency Virus (SIV), Moloney Leukemia Virus (MLV) and Human Immunodeficiency Viruses (HIV-1 and HIV-2) especially Human Immunodeficiency Virus of type 1 (HIV-1).

In some embodiments, a virus-like particle may also comprise one or more viral envelope protein(s). The presence of one or more viral envelope protein(s) may impart to the said virus-derived particle a more specific tropism for the cells which are targeted, as it is known in the art. The one or more viral envelope protein(s) may be selected in a group comprising envelope proteins from retroviruses, envelope proteins from non-retroviral viruses, and chimeras of these viral envelope proteins with other peptides or proteins. An example of a non-lentiviral envelope glycoprotein of interest is the lymphocytic choriomeningitis virus (LCMV) strain WE54 envelope glycoprotein. These envelope glycoproteins increase the range of cells that can be transduced with retroviral derived vectors.

### Pharmaceutical Compositions

Other aspects of the present disclosure relate to pharmaceutical compositions comprising any of the various prime editing system described herein (e.g., including, but not limited to, the napDNAbps, reverse transcriptases, fusion proteins (e.g., comprising napDNAbps and reverse transcriptases), extended guide RNAs, and complexes comprising fusion proteins and extended guide RNAs, as well as accessory elements, such as second strand nicking components and 5′ endogenous DNA flap removal endonucleases for helping to drive the multi-flap prime editing process towards the edited product formation).

In some embodiments, the pharmaceutical composition further comprises a pharmaceutically acceptable carrier. In some embodiments, the pharmaceutical composition comprises additional agents (e.g. for specific delivery, increasing half-life, or other therapeutic compounds).

Some examples of materials which can serve as pharmaceutically-acceptable carriers include, but are not limited to: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil: (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid: (16) pyrogen-free water, (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105). Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierce-able by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### REFERENCES

Mort, M., Ivanov, D., Cooper, D. N. & Chuzhanova, N. A. A meta-analysis of nonsense mutations causing human genetic disease. Hum Mutat 29, 1037-1047 (2008).
Karijolich, J. & Yu. Y. T. Therapeutic suppression of premature termination codons: mechanisms and clinical considerations (review). Int J Mol Med 34, 355-362 (2014).
Banskota, S. et al. Engineered virus-like particles for efficient in vivo delivery of therapeutic proteins. Cell 185, 250-265 e216 (2022).
Krishnamurthy, S. et al. Functional correction of CFTR mutations in human airway epithelial cells using adenine base editors. Nucleic Acids Res 49, 10558-10572 (2021).
Osborn, M. J. et al. Base Editor Correction of COL7A1 in Recessive Dystrophic Epidermolysis Bullosa Patient-Derived Fibroblasts and iPSCs. J Invest Dermatol 140, 338-347 e335 (2020).
Porter, J. J., Heil, C. S. & Lueck, J. D. Therapeutic promise of engineered nonsense suppressor tRNAs. Wiley Interdiscip Rev RNA 12, e1641 (2021).
Liu, C. C. & Schultz, P. G. Adding new chemistries to the genetic code. Annu Rev Biochem 79, 413-444 (2010).
Wang, J. et al. AAV-delivered suppressor tRNA overcomes a nonsense mutation in mice. Nature 604, 343-348 (2022).
Lueck, J. D. et al. Engineered transfer RNAs for suppression of premature termination codons. Nat Commun 10, 822 (2019).
Buvoli, M., Buvoli, A. & Leinwand, L. A. Suppression of nonsense mutations in cell culture and mice by multimerized suppressor tRNA genes. Mol Cell Biol 20, 3116-3124 (2000).
Torres, A. G., Reina, O., Stephan-Otto Attolini, C. & Ribas de Pouplana. L. Differential expression of human tRNA genes drives the abundance of tRNA-derived fragments. Proc Natl Acad Sci USA 116, 8451-8456 (2019).
Iben, J. R. & Maraia, R. J. tRNA gene copy number variation in humans. Gene 536, 376-384 (2014).
Berg, M. D. & Brandl, C. J. Transfer RNAs: diversity in form and function. RNA Biol 18, 316-339 (2021).
Himeno, H., Yoshida, S., Soma, A. & Nishikawa, K. Only one nucleotide insertion to the long variable arm confers an efficient serine acceptor activity upon Saccharomyces cerevisiae tRNA(Leu) in vitro. J Mol Biol 268, 704-711 (1997).
Anzalone, A. V. et al. Search-and-replace genome editing without double-strand breaks or donor DNA. Nature 576, 149-157 (2019).
Anzalone, A. V. et al. Programmable deletion, replacement, integration and inversion of large DNA sequences with twin prime editing. Nat Biotechnol 40, 731-740 (2022).
Chan, P. P. & Lowe, T. M. GtRNAdb 2.0: an expanded database of transfer RNA genes identified in complete and draft genomes. Nucleic Acids Res 44, D184-189 (2016).
Nelson, J. W. et al. Engineered pegRNAs improve prime editing efficiency. Nat Biotechnol (2021).
Doman, J. L., Sousa, A. A., Randolph, P. B., Chen, P. J. & Liu, D. R. Designing and executing prime editing experiments in mammalian cells. Nat Protoc 17, 2431-2468 (2022).
Duvoisin, R. et al. Human U6 promoter drives stronger shRNA activity than its schistosome orthologue in Schistosoma mansoni and human fibrosarcoma cells. Transgenic Res 21, 511-521 (2012).
Yarnall, M. T. N. et al. Drag-and-drop genome insertion of large sequences without double-strand DNA cleavage using CRISPR-directed integrases. Nat Biotechnol (2022).
Durrant, M. G. et al. Systematic discovery of recombinases for efficient integration of large DNA sequences into the human genome. Nat Biotechnol (2022).
Klompe, S. E., Vo, P. L. H., Halpin-Healy, T. S. & Sternberg, S. H. Transposon-encoded CRISPR-Cas systems direct RNA-guided DNA integration. Nature 571, 219-225 (2019).
Strecker, J. et al. RNA-guided DNA insertion with CRISPR-associated transposases. Science 365, 48-53 (2019).
Tou, C. J. & Kleinstiver, B. P. Recent Advances in Double-Strand Break-Free Kilobase-Scale Genome Editing Technologies. Biochemistry (2022).
Chen, P. J. & Liu, D. R. Prime editing for precise and highly versatile genome manipulation. Nat Rev Genet (2022).
Clarke, L. A. et al. The effect of premature termination codon mutations on CFTR mRNA abundance in human nasal epithelium and intestinal organoids: a basis for read-through therapies in cystic fibrosis. Hum Mutat 40, 326-334 (2019).
Buckley, R. H. The multiple causes of human SCID. J Clin Invest 114, 1409-1411 (2004).
Chen, P. J. et al. Enhanced prime editing systems by manipulating cellular determinants of editing outcomes. Cell 184, 5635-5652.e5629 (2021).
Anzalone, A. V., Koblan, L. W. & Liu, D. R. Genome editing with CRISPR-Cas nucleases, base editors, transposases and prime editors. Nat Biotechnol 38, 824-844 (2020).
Gaudelli, N. M. et al. Programmable base editing of A*T to G*C in genomic DNA without DNA cleavage. Nature 551, 464-471 (2017).
Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
Newby, G. A. & Liu, D. R. In vivo somatic cell base editing and prime editing. Mol Ther 29, 3107-3124 (2021).
Rees, H. A. & Liu, D. R. Base editing: precision chemistry on the genome and transcriptome of living cells. Nat Rev Genet 19, 770-788 (2018).
Koblan, L. W. et al. Efficient C*G-to-G*C base editors developed using CRISPRi screens, target-library analysis, and machine learning. Nat Biotechnol 39, 1414-1425 (2021).
Nishimasu, H. et al. Engineered CRISPR-Cas9 nuclease with expanded targeting space. Science 361, 1259-1262 (2018).
Neugebauer, M. E. et al. Evolution of an adenine base editor into a small, efficient cytosine base editor with low off-target activity. Nat Biotechnol (2022).
Berg, M. D. et al. Targeted sequencing reveals expanded genetic diversity of human transfer RNAs. RNA Biology (2019).

### EXAMPLES

### Example 1. Prime Editing Conversion of Endogenous tRNAs to Suppressor tRNAs in HEK293T Cells

A panel of epegRNAs and nicking guide RNAs were designed targeting the indicated human tRNA genes. These epegRNA and nicking guide RNA pairs were transiently delivered to HEK293T cells by plasmid transfection. Seventy-two hours post-transfection, genomic DNA was harvested and editing efficiency was determined by amplicon sequencing of the targeted tRNA gene. The plotted data represents the highest editing efficiency achieved for each targeted tRNA gene.

### Example 2. Demonstration of PERT Using an eGFP Reporter Assay

To demonstrate the validity of PERT, epegRNAs and nicking guide RNAs were designed targeting two endogenous tRNAs, Arg-CCG-2-1 and Leu-TAA-2-1, to effectuate mutations in their anticodons to CTA and TCA, respectively. These epegRNAs and nicking guide RNAs were delivered alongside an optimized prime editor enzyme to HEK293T cells. Forty-eight hours after the editing components were delivered, a reporter plasmid encoding an eGFP cassette with a PTC was transfected into the edited cells and unedited control cells (FIG. 3A). The frequency of cells exhibiting readthrough was quantified using fluorescence-activated cell sorting (FACS) and editing efficiency was quantified using amplicon sequencing (FIG. 3B,C). In Arg-CCG-2-1 and Leu-TAA-2-1 edited cells, fluorescent signal was 1.75% and 13.13% of wild type eGFP control cell populations, respectively (FIG. 3C).

### Example 3. Approaches to Identify the Most Effective Suppressor tRNA Gene

While the anticodon loop of any tRNA gene can theoretically be altered to bind a premature amber, ochre, or opal stop codon, the ability of each suppressor tRNA gene to enable readthrough is not equivalent. Variables that can change suppressor tRNA efficiency include: tRNA expression levels, correct placement of RNA modifications that can modify tRNA stability, correct processing of the 5′ and 3′ ends of the tRNA to facilitate activity and localization, and adequate recognition by the appropriate synthetase enzyme for charging the tRNA.

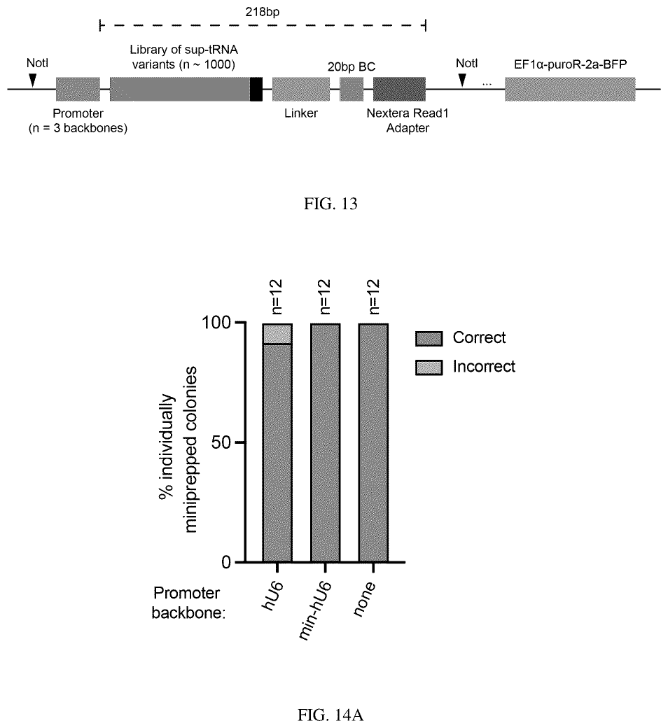

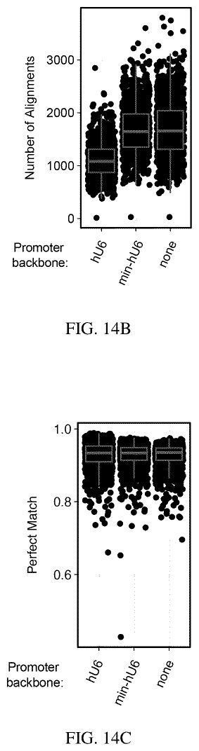

To identify the most effective suppressor tRNA gene to install into the genome, a strategy was developed to screen suppressor tRNA sequences in high throughput. First, a reporter cell line was constructed that constitutively expresses an mCherry fluorescent protein followed by a PTC (either TGA, TAG, or TAA), a ribosomal skipping element, and a GFP fluorescent protein (FIG. 13). At baseline, this reporter cell line expresses the mCherry protein only; however, following successful readthrough using a suppressor tRNA with an anticodon loop complementary to the given PTC, the cell will express both mCherry and GFP proteins. To more closely mimic a therapeutic setting in which only one or two alleles of a single gene harbor a nonsense mutation, a cell line was designed such that each cell expressed only a single-copy of the PTC-containing reporter. Control experiments were also conducted to confirm that the PTC in this reporter construct could be replaced with any amino acid variant without altering GFP expression levels, which theoretically enables any suppressor tRNA variant to directly be compared to each other based on the percentage of cells exhibiting GFP readthrough (FIGS. 14A-14D).

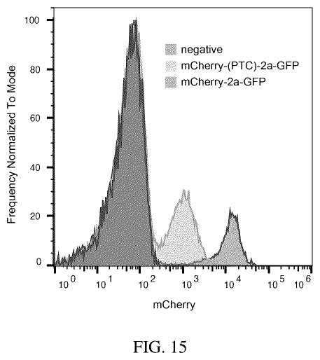

Next an initial set of suppressor tRNA variants were designed by taking all mature human tRNA gene sequences and replacing their native 3-bp anticodon with an anticodon that recognizes the amber stop codon (e.g., codon TAG, which corresponds to the anticodon CTA). To assess the minimal promoter elements required to express a suppressor tRNA, this pool of tRNA variants were cloned into lentiviral backbones containing a human U6 promoter (249-bp), a minimal human U6 promoter sequence lacking a nucleosome positioning element (111-bp), or no exogenous promoter (0-bp) (FIG. 15). tRNAs contain endogenous polIII promoter elements that theoretically should be sufficient to position polIII polymerase without the need for an exogenous promoter. All suppressor tRNAs in the screen were immediately followed by a sequence of seven thymidines, which signals polIII polymerase to stop transcription.

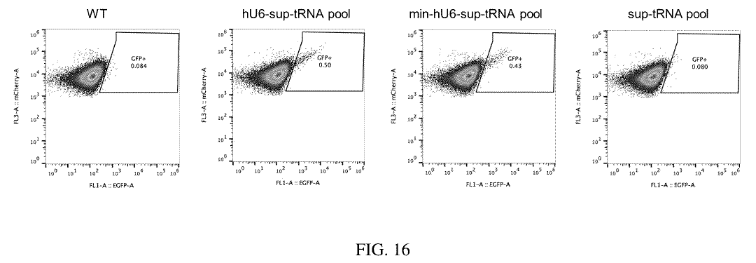

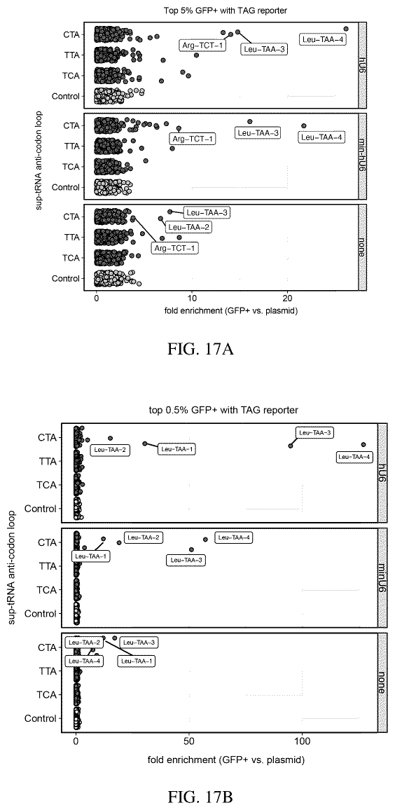

Control experiments were conducted to confirm the fidelity of the candidate suppressor tRNA screening plasmids (FIGS. 16A-16C). Interestingly, protein expression levels of PTC-containing transcripts expressed from a lentiviral construct were >10-fold lower than non-PTC containing transcripts (FIG. 17, e.g., transcripts that were successfully readthrough using suppressor tRNAs). Without wishing to be bound by any particular theory, it is generally believed that nonsense-mediated decay (NMD) machinery is thought to be recruited to PTC-containing mRNAs with (1) downstream splicing factors and/or (2) long 3′ UTRs. Lentiviral sequences do not typically have polyA tails, which would truncate packaging of the RNA virus).

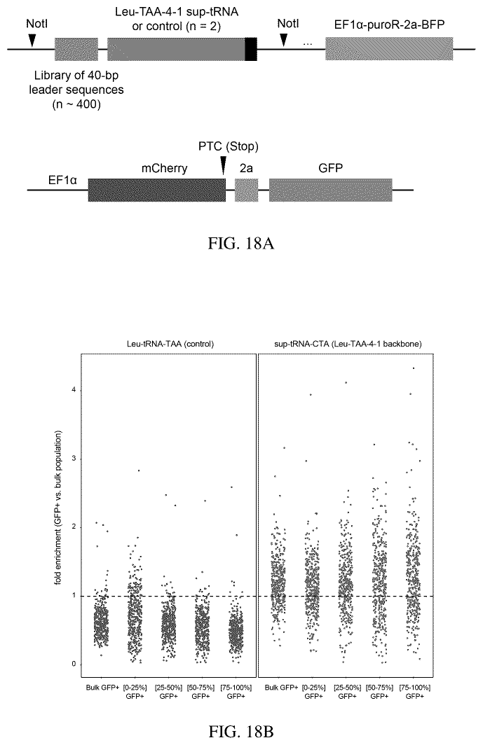

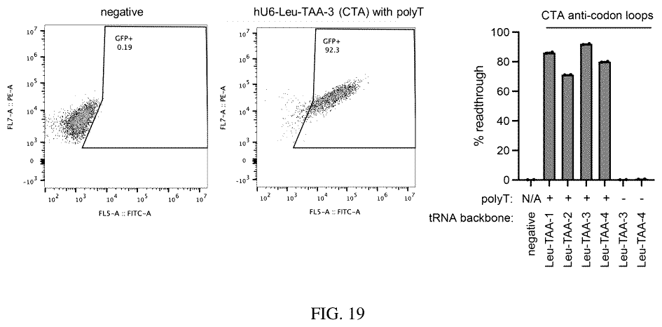

The lentiviral library of tRNA sequences was introduced into the reporter cell line at a single-copy per cell. Fluorescence-activated cell sorting (FACS) was used to sort cells (FIG. 18) that successfully read through the PTC and expressed the GFP protein followed by next generation sequencing to identify which tRNA sequences led to the highest levels of readthrough (FIG. 19A, top 5% and FIG. 19B, top 0.5%). The suppressor tRNA variants that led to the strongest readthrough levels (e.g., top 0.5%) with this design were the four members of the Leu-TAA family of tRNAs with CITA anticodon loops (Leu-TAA-1-1, Leu-TAA-2-1, Leu-TAA-3-1, and Leu-TAA-4-1) (FIG. 19B). Interestingly, while the endogenous promoter of the tRNA was technically sufficient to enable readthrough of the reporter, the enrichment values for tRNAs driven by an endogenous promoter were several orders of magnitude weaker than for tRNAs driven by a human U6 promoter.

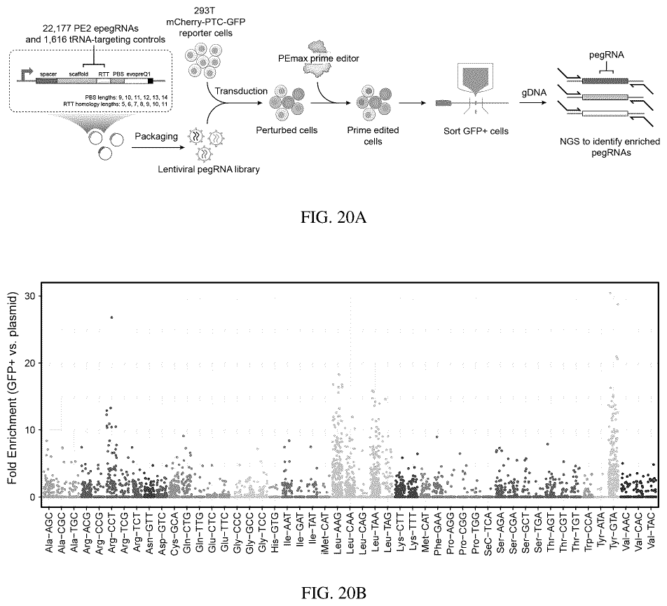

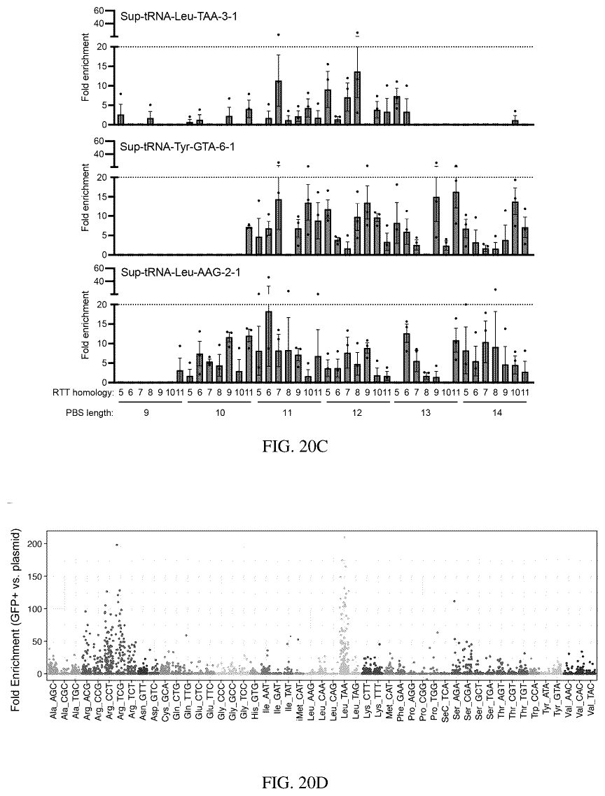

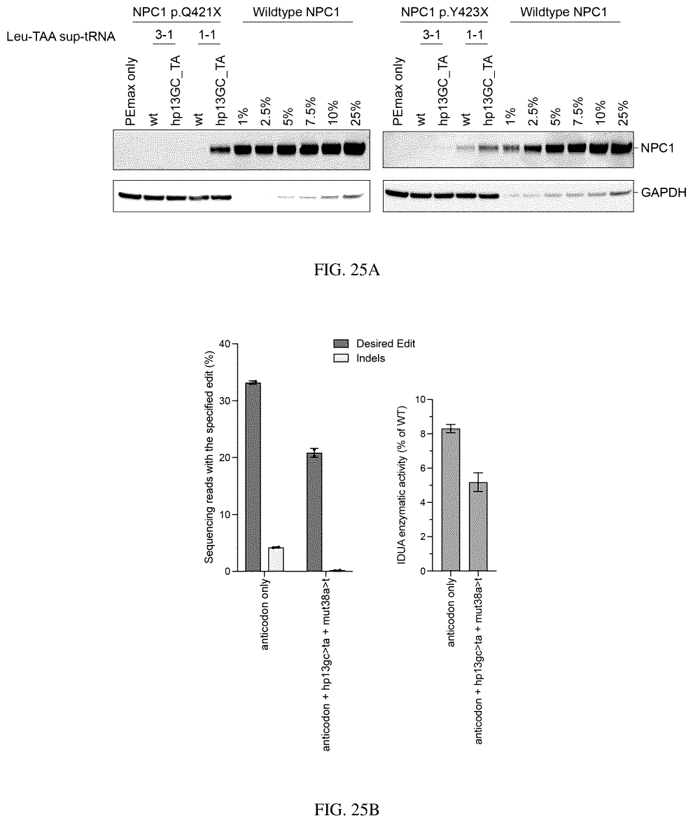

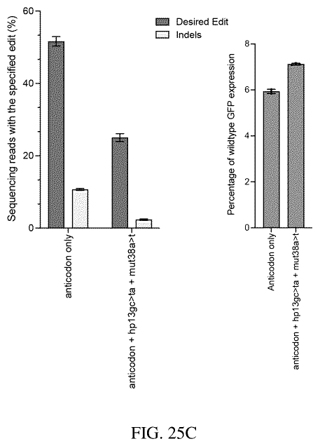

Given that tRNAs are among the most highly expressed RNAs in the genome, it was hypothesized that tRNAs might have additional surrounding sequences that regulate their expression and that therefore might impact their aptitude when they are converted to suppressor tRNAs. In particular, the 40-bp sequences upstream of each mature tRNA in the human genome are highly diversified sequences of unknown function. To evaluate the ability of these sequences to modulate expression levels of potential suppressor tRNAs, a library of all 40-bp sequences that precede each mature tRNA sequence in the human genome was cloned in front of either one of the top suppressor tRNA hits in the original screen (Leu-TAA-4-1 with a CTA anticodon) or a control tRNA (Leu-TAA-4-1 with a TAA anticodon) (FIG. 20A). Surprisingly, nearly all 40-hp sequences were sufficient to enable readthrough of the reporter gene using the endogenous tRNA promoter, to levels that are on par with or even better than with a 249-bp exogenous human U6 promoter (FIG. 20B). This finding suggests that the endogenous promoter of a suppressor tRNA is sufficient to drive expression of the tRNA, as long as an upstream sequence element is encoded alongside the tRNA sequence. Also, it was found that the best leader sequences precede highly expressed tRNAs (FIG. 25).

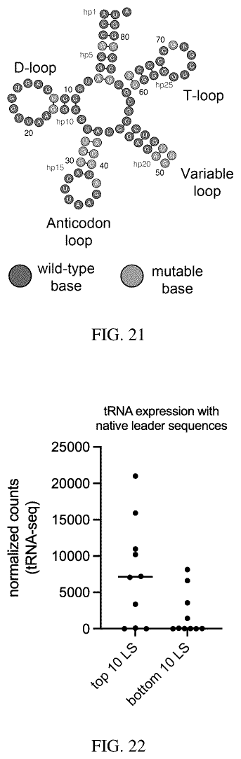

Since the sequence preceding a tRNA is important for regulating its expression, it was hypothesized that a termination sequence may also be required element of the tRNA sequence. It is known in the art, that four and five thymidine tracks are sufficient to initiate termination with 25% and 99% efficiency, respectively. Interestingly, 28/419 (6.7%) high-confidence human tRNA sequences do not have a four thymidine track and 217/419 (51.8%) do not have a five thymidine track within 100-bp of the mature tRNA sequence. When the Leu-TAA-3-1 and Leu-TAA-4-1 suppressor tRNAs with CTA anticodon loops are expressed without an immediately adjacent polyT sequence, readthrough efficiency is abolished (FIG. 21). Therefore, a termination sequence appears to be an essential feature of a suppressor tRNA. Without wishing to be bound by any particular theory, it is generally believed that the termination sequence facilitates recycling of polIII back to the promoter and thereby influences tRNA expression, which is a mechanism that has been shown to greatly accelerate polIII-driven transcription in in vitro yeast studies.

To evaluate the potential of converting endogenous tRNA sequences into suppressor tRNA sequences, >22,000 prime editing guide RNAs (pegRNAs) targeting every tRNA sequence in the genome and which convert the natural tRNA anticodon to CTA to recognize the amber stop codon were designed (FIG. 22A). Of note, since this screen targets the anticodon of tRNAs at their endogenous loci, the leader sequence and termination sequence of the endogenous tRNA remains unchanged from the original sequence context. Approximately 2,000 tRNA-targeting controls which convert native tRNA anticodons to other anticodons of the same isoacceptor family were also included, which should have a neutral effect. To screen which pegRNAs worked best, pegRNAs were cloned as a pool into a lentiviral backbone and transduced into reporter cells such that each cell expressed one pegRNA sequence. Cell were then transfected with an optimized prime editor protein (PEmax), cells that read through the reporter PTC and expressed GFP protein were sorted, and next generation sequencing used to identify which pegRNAs were enriched in the GFP+ population. Of note, the pegRNAs enriched in this screen represent pegRNAs that not only mediated successful editing of the anticodon loop of the targeted tRNA but also that resulted in a functional suppressor tRNA capable of readthrough. In comparison to the screen expressing suppressor tRNAs off of a lentiviral construct with an exogenous promoter, this screen resulted in additional hits from other tRNA families, including representative members from the Arg-CCT, Leu-AAG, Leu-CAA, Leu-TAA, Leu-TAG, and Tyr-GTA tRNA families (FIGS. 22B and 22C). The above discussion and exemplary embodiments are not limited to amber stop codon. Similar screens to create suppressor tRNAs with anticodons corresponding to the opal and ochre stop codons using the appropriate reporter cell line, as also herein contemplated. A similar screen was performed in a reporter cell line harboring a TGA stop codon to create suppressor tRNAs with anticodons corresponding to the opal stop codon, which revealed both overlapping and distinct subsets of suppressor tRNAs (FIG. 22D).

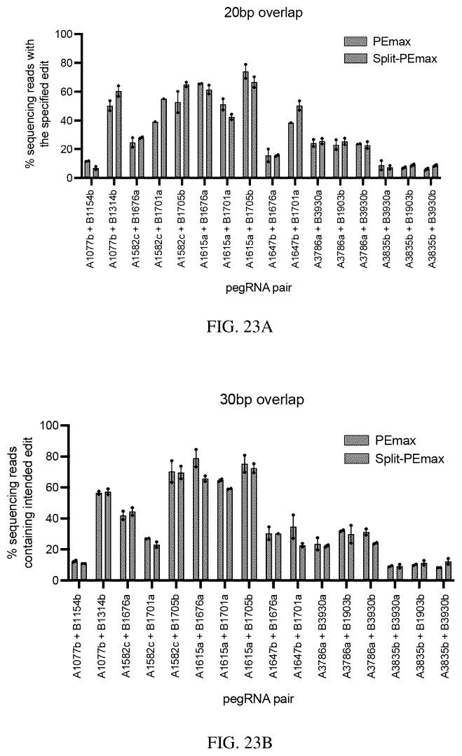

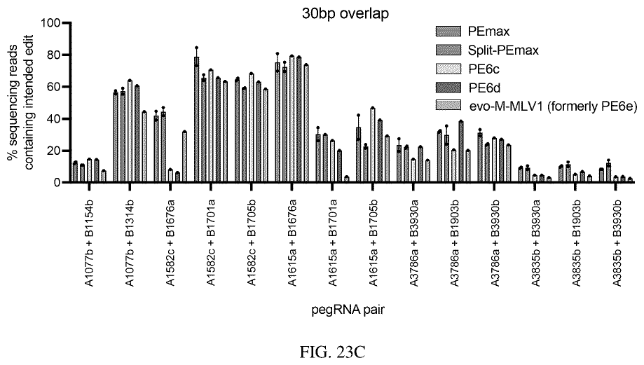

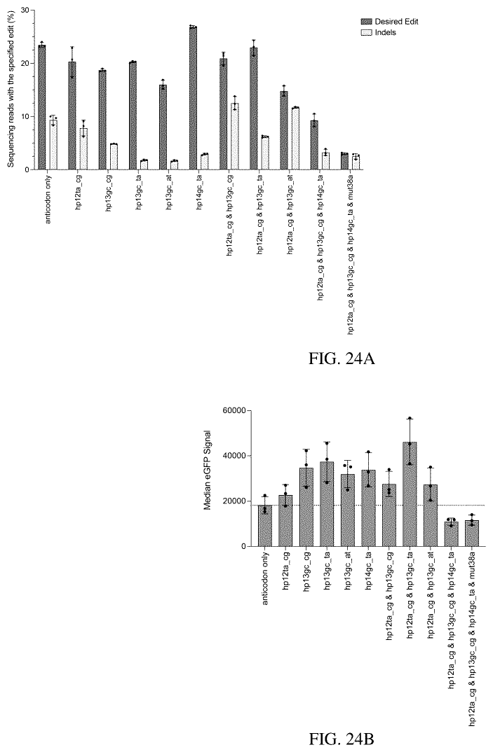

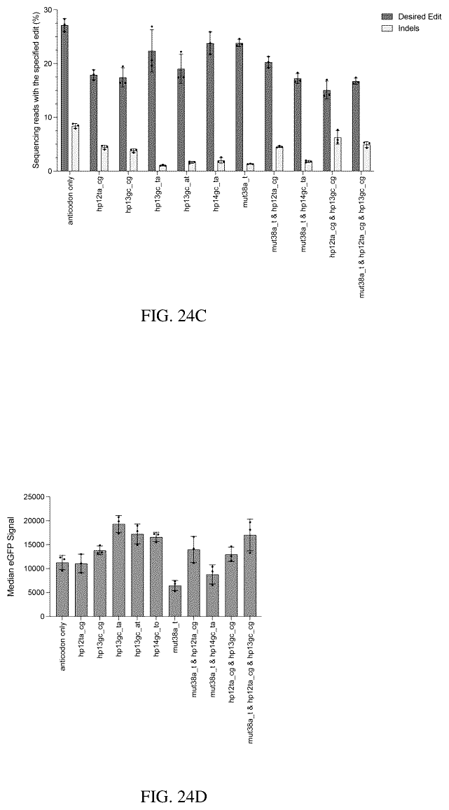

While the screens described so far have used existing tRNA backbones for suppressor tRNA sequences, wherein only the anticodon is changed relative to the wild-type tRNA sequence, we hypothesized that adding additional mutations could increase the effectiveness of a suppressor tRNA. In addition, the ability to add neutral mutations to the tRNA sequence while editing the anticodon loop could prevent the prime editor from re-binding and re-nicking the sequence after the correct edit has been made, which could have implications for maintaining high expression levels of the tRNA. As a proof of concept, we took one of the highest performing suppressor tRNAs from the original lentiviral screen (Leu-TAA-4-1 with the anticodon loop changed to CTA, see FIG. 19B) and performed saturation mutagenesis across the entire 83-bp sequence (FIG. 23). We created a library of every single base pair variant, every single base pair deletion, and every hairpin double mismatch variant, cloned these sequences into a lentiviral backbone, transduced the reporter cells, sorted cells that expressed GFP protein, and used next generation sequencing to identify the highest performing variants capable of readthrough. Of the 534 variant sequences, 21 were capable of stronger readthrough than the wild-type suppressor tRNA sequence, with the most highly enriched variant leading to >60% greater enrichment than wild-type (FIG. 23). Without being bound by any particular theory, it is believed that the combination of these variants may further improve the performance of this suppressor tRNA. The above discussion and exemplary embodiments are not limited to the Leu-TAA-4-1 tRNA. Similar screens to create other mutated suppressor tRNAs with enhanced performance are also contemplated herein.

### Example 4. Prime Editing Approaches to Edit an Endogenous tRNA Gene

To perform simple anticodon edits, standard prime editing, as opposed to twin prime editing, is sufficient. Due to prime editing's exquisite target specificity, pegRNAs can be carefully designed to distinguish between on-target and off-target tRNA genes, even ones that differ at most by a few base pairs. Alternatively, in the case of large families of tRNAs with many redundant and dispensable family members, prime editing can be used to edit multiple tRNA family members with the same sequence to maximize expression and potential readthrough efficiency. PERT can be applied to convert any of the human tRNA genes (listed in Table 1) into suppressor tRNAs, as we demonstrated with a ˜24,000 element pegRNA screen to convert endogenous tRNAs into amber suppressor tRNAs (FIG. 22). Additional example pegRNA sequences that could convert these human tRNAs into amber, opal, or ochre suppressor tRNAs are listed in Table 2. Stabilizing sequences can be appended to the end of each pegRNA in Table 2 (resulting in “epegRNAs”) in order to increase prime editing efficiencies.

While many possible pegRNAs could in principle be used to mediate the above-described edits, the specific pegRNA(s) need to be optimized for high-efficiency prime editing. Once particular pegRNAs have been optimized for a particular locus (either an endogenous tRNA locus or a safe harbor locus), those pegRNAs, along with an optimal prime editor variant, form a single composition of matter that could be used to rescue all diseases caused by a specific type of premature stop codon. This underscores a major advantage of PERT compared to existing methods: editing agent optimization is required only once during the research and development phase, yielding a generalizable therapeutic strategy for multiple diseases.

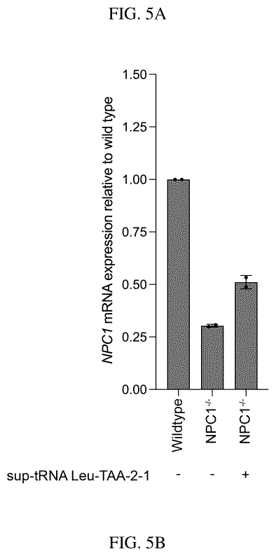

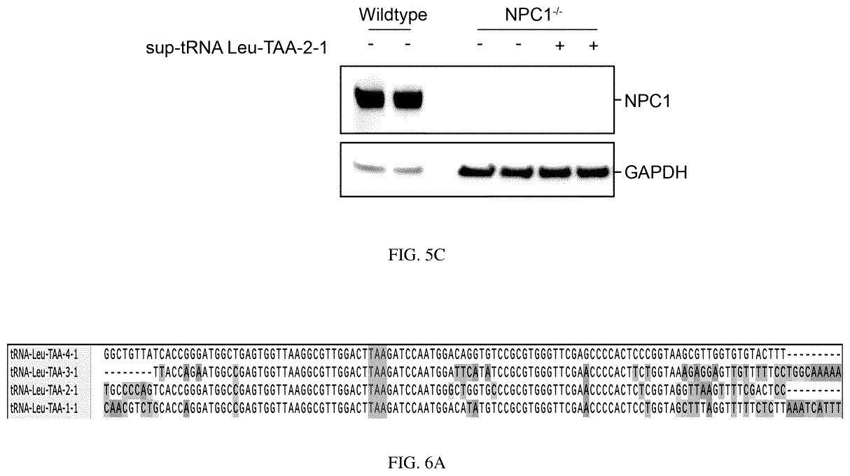

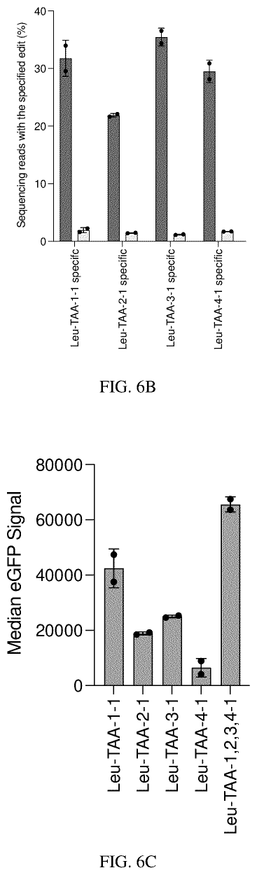

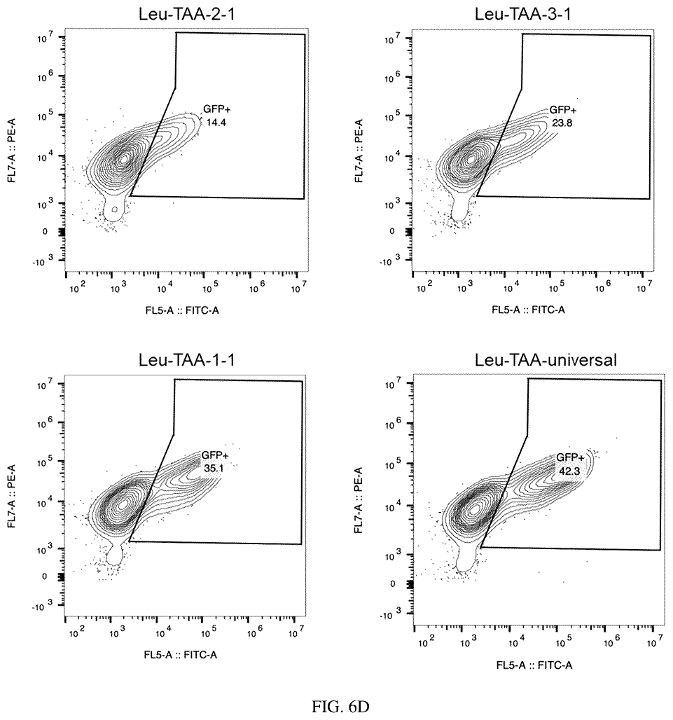

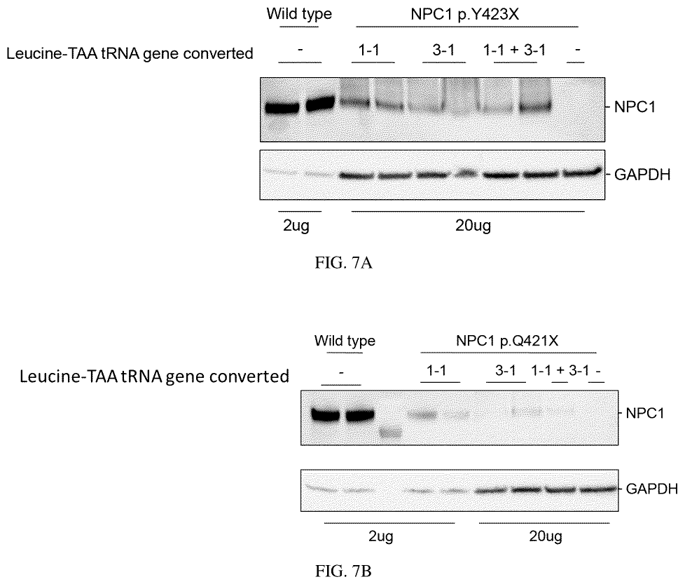

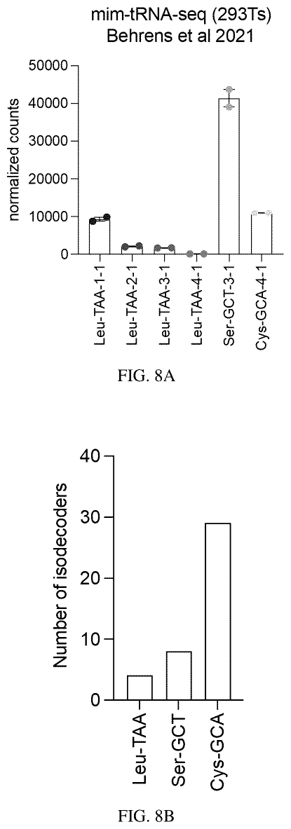

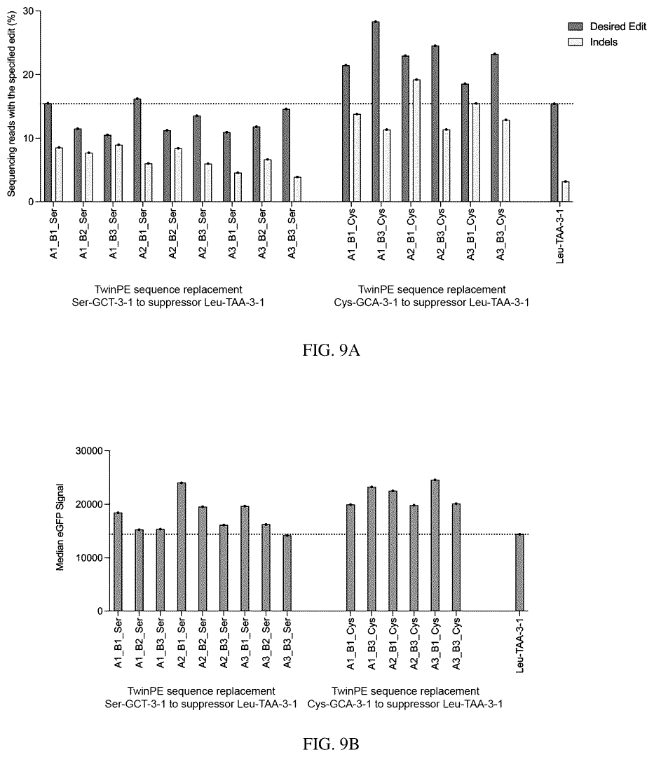

To demonstrate the feasibility of PERT, a set of epegRNA and nicking guide RNA pairs targeting each of the Leu-TAA tRNA genes were designed. Prime editing of Leu-TAA-2-1 led to detectable read through at mRNA but not protein levels (FIG. 5A-5C). Prime editing of Leu-TAA-1-1. Leu-TAA-2-1, and Leu-TAA-3-1 led to detectable readthrough in the reporter cell line described above (FIG. 6A-6D). Editing resulted between 20% and 40% of the sequencing reads with the specified edit and between 0% and 2.5% indels. Results were confirmed using readthrough of a single-copy integrated endogenous eGFP reporter assay, which was quantified using flow cytometry (FIGS. 6C and 6D). Prime editing of Leu-TAA-4-1, however, did not lead to detectable readthrough, which is likely explained by low endogenous expression of this tRNA in HEK293T cells (FIG. 6B). We then made two clonal HEK293T cell models harboring homozygous PTCs at two different positions in the NPC1 gene (NPC1 p.Y423X and NPC1 p.Q421X). Using these cell models, we delivered a prime editor enzyme alongside the epegRNA and nicking guide RNA pairs targeting either Leu-TAA-1-1, Leu-TAA-3-1 or both simultaneously, converting them into suppressor tRNAs. In each condition, the PERT treatment of these cell models led to rescue of western blot-detectable full-length NPC1 protein (FIGS. 7A and 7B). These results support PERT as a viable strategy to elicit PTC readthrough of an endogenous disease locus and demonstrate the generalizability of the approach to distinct PTCs.

The versatility of prime editing allows for the conversion of larger sequences, which permits the introduction of additional changes beyond simple anticodon editing. To demonstrate this, PERT was used to preliminarily validate a subset of the variant sequences identified to enhance readthrough efficiency in the saturation mutagenesis screen described above. Since the Leu-TAA-4-1 tRNA gene that was used as a backbone for the saturation mutagenesis screen is not sufficiently expressed in HEK293T cells, the chosen variants were introduced into the Leu-TAA-3-1 or Leu-TAA-1-1 endogenous tRNA genes. Without being bound by any particular theory, it is believed that the high sequence similarity found between the Leu-TAA family members would allow for the beneficial impact of the candidate variant sequences to be generally applicable to any Leu-TAA tRNA gene. epegRNA and ngRNA pairs were designed to introduce a subset of the variant sequences either in isolation or in combinations with each other. epegRNA and ngRNA pairs were transfected with a PEmax enzyme to the HEK293T reporter cell line. Multiple hits from the saturation mutagenesis screen led to enhanced readthrough when introduced into both the Leu-TAA-1-1 and Leu-TAA-3-1 endogenous tRNA genes (FIG. 26). epegRNA and ngRNA pairs capable of introducing a change of hp13 from G•C to T•A—a top hit from the validation in the reporter cell line-were delivered to HEK293T Niemann-Pick disease type C cells and the readthrough efficiency measured by western blot. The introduction of the hairpin change alongside the anticodon edit led to a marked increase in full-length NPC1 protein production, reaching approximately 1% of wildtype control expression (FIG. 27).

To further demonstrate the versatility of PERT, the murine Leu-TAA-2-1 gene was edited in a cell model recapitulating the human IDUA p.W402X mutation underlying mucopolysaccharidosis type I. pegRNAs and ngRNAs were delivered to convert the anticodon only or the anticodon alongside the murine equivalent of three additional mutations identified by the saturation mutagenesis screen. Using a fluorometric enzyme activity assay, the average translational readthrough of up to 8% of wildtype IDUA protein was measured (FIG. 27B). Furthermore, to preliminarily validate the epegRNA screen data performed in the context of a TGA stop codon, cells were transfected with a TGA fluorescent reporter cell line equivalent to the TAG reporter described above with PERT reagents. An edit was introduced that converted the anticodon alone or the anticodon alongside three additional mutations identified in the saturation mutagenesis screen. These edits led to average readthrough of 5.93% and 7.12%, respectively (FIG. 27C).

Together, these data support applications of PERT in converting endogenous tRNAs into engineered, highly active suppressor tRNAs while validating the screening methodology described above.

### Example 5. Prime Editing Approaches to Replace an Endogenous tRNA Gene

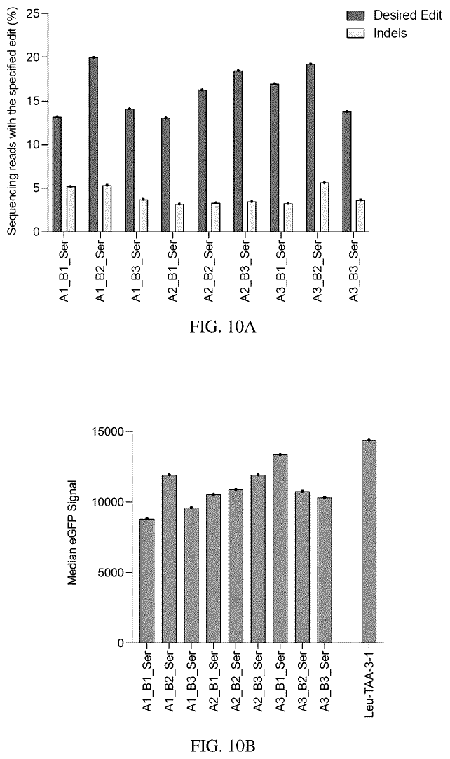

Given the complex factors governing suppressor tRNA efficiency and that repurposing certain tRNAs into suppressor tRNAs might be negatively perturbative to cell function, overwriting an existing tRNA with a suppressor tRNA could be more effective than simple anticodon editing. For example, an ideal tRNA candidate for conversion to a suppressor tRNA may not be expressed in a target tissue (FIG. 10A), and thus would likely elicit poor levels of readthrough. To address this, a highly expressed tRNA gene could be overwritten with the sequence of an optimized suppressor tRNA candidate sequence. This is possible as many tRNAs have a plurality of isodecoders that can be edited (FIG. 10B). This approach negates the need to introduce additional sequence elements, given our data demonstrating that the upstream sequence element can support expression of non-native tRNA sequences.

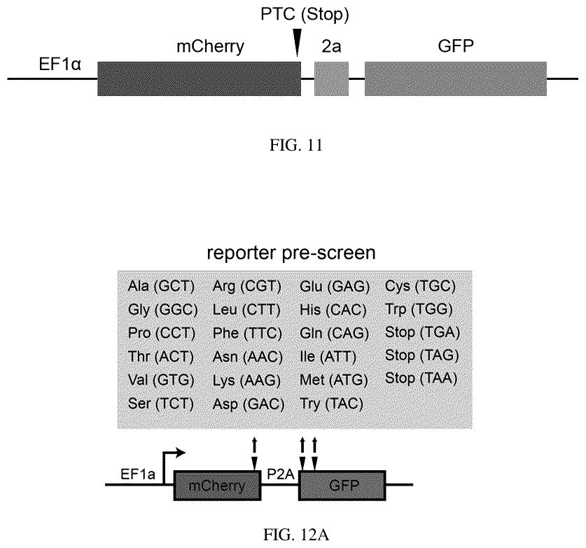

Replacing an endogenous tRNA with a suppressor tRNA could be achieved by standard prime editing, though the length of the sequence replacement might limit editing efficiency. Dual-flap prime editing, or twin prime editing, may provide higher editing efficiencies. To illustrate this approach, a pair of twin prime editing epegRNAs targeting two tRNA genes shown to be highly expressed in HEK293T cells, Cys-GCA-4-1 and Ser-GCT-3-1, were designed. These twin prime editing epegRNA pairs were designed to replace the native tRNA sequence with the sequence of Leu-TAA-3-1 with a modified anticodon to suppress amber stop codons. epegRNAs were transfected alongside the PEmax enzyme into the HEK293T reporter cell line. Twin prime editing efficiencies ranged from 10.54% to 28.32% and showed similar or modestly improved levels of readthrough compared to simple anticodon editing of the endogenous Leu-TAA-3-1 gene (FIG. 11A). FIG. 11B shows results using a second assay based on readthrough of a single-copy integrated endogenous eGFP reporter assay quantified using flow cytometry. Importantly, the general trends observed using the reporter assay system mirrored those obtained via sequencing (e.g., A3_B1_Cys exhibited the greatest signal followed by A2_B1_Ser, A1_B1_Ser, and A3_B3_Ser). Since the twin prime editing epegRNAs encode the entire suppressor tRNA sequence, this approach could be adapted to replace existing tRNA genes with highly engineered, sequence-modified suppressor tRNAs optimized for maximum readthrough. This strategy allows for additional flexibility in PERT applications, including allowing for minimally perturbative or tissue-specific suppressor tRNA expression.

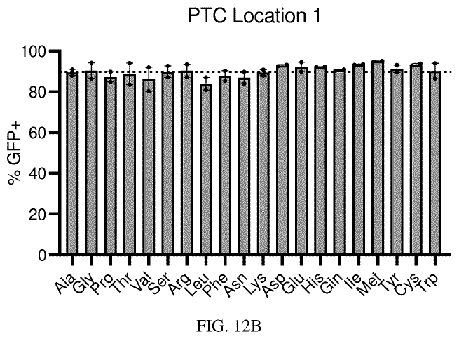

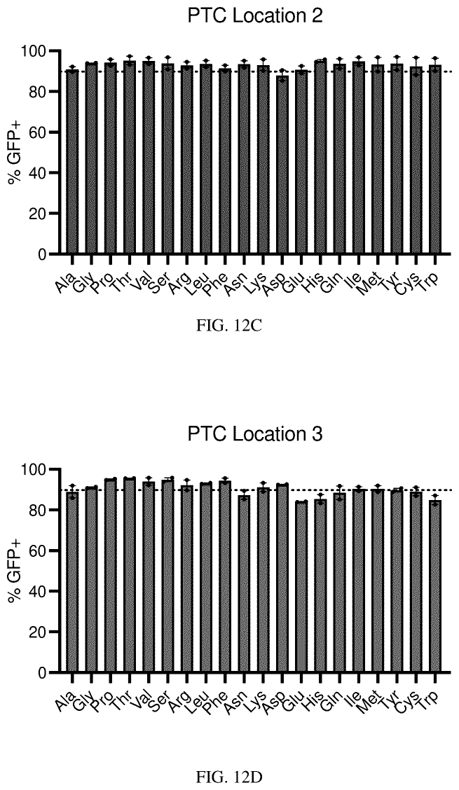

Next, the ability to rescue the suppressor activity of sup-tRNA-Leu-TAA-4-1 was rescued by writing the gene into tRNAs with highly expressed loci. As previously described elsewhere herein, twinPE was used to insert the tRNA-TAA-4-1 gene into the loci of various Ser-GCT-3-1 isodecoders. FIG. 12A shows a plot of the percentage of sequencing reads with the specified edit and indels as a function of the various edited isodecoders. The percentage of reads was between 10% and 20% for all sup-tRNA-TAA-4-1 constructs. FIG. 12B shows results using a second assay based on readthrough of a single-copy integrated endogenous eGFP reporter assay quantified using flow cytometry. Importantly, the general trends observed using the reporter assay system mirrored those obtained via sequencing with median eGFP signals that were at least 2-3× greater than suppressor tRNAs obtained from editing at the Leu-TAA-4-1 locus (FIG. 6C).

### Example 6. Prime Editing Approaches to Insert a Suppressor tRNA into a Safe Harbor Locus

An alternative approach to PERT involves the insertion of a new suppressor tRNA gene into a safe harbor locus or general expression site in the human genome (such as ROSA26 or ALB), rather than converting an endogenous tRNA gene into a suppressor tRNA. This approach requires insertion of a small gene rather than a local edit of a subset of endogenous tRNA bases, but may offer complementary advantages such as the lack of dependence on the presence, sequence, and dispensability of an endogenous tRNA gene in a specific target organism or patient. Because tRNAs are short (˜80 bp6) and encode their own Pol III promoter elements within the body of the tRNA sequence combined with short leading sequences (˜40 bp), it is possible that all of the elements required for suppressor tRNA expression could be inserted by prime editing methods such as twin prime editing26. To demonstrate this, a panel of twin prime editing epegRNAs targeting the putative safe harbor loci within the genome were designed and tested. Results showed that the mature Gln-CTG-5-1 tRNA sequence could be edited into the AAVS1 locus with editing efficiencies of up to 80% (FIG. 25A-25C). The above discussion and exemplary embodiments are not limited to the Gln-CTG-5-1 tRNA sequence or the AAVS1 locus. Embodiments directed toward the introduction of any suppressor tRNA sequence any suitable safe harbor loci are herein contemplated.

Alternatively, prime editing or twin prime editing coupled with integrase or recombinase enzymes could be used to perform the insertion. The use of CRISPR-associated transposases (CASTs) and other targeted gene insertion technologies to achieve insertion of a suppressor tRNA or a suppressor tRNA expression cassette into the human genome is likewise also envisioned.

Without wishing to be bound by theory, it is generally believed that because prime editing is efficient in numerous cell types and in mice, PERT will be able to promote therapeutic stop codon readthrough in all of these cell types. Additionally, PERT can be applied both for ex vivo and in vivo therapies. Devastating diseases such as cystic fibrosis and severe combined immunodeficiency disease can be caused by numerous different premature stop codons at different locations within a particular gene, and a single PERT strategy may be used to treat all of these diseases. For these reasons, PERT could transform the landscape of therapies for genetic diseases caused by premature stop codons.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim may be modified to include one or more limitations found in any other claim that is dependent on the same base claim. Where elements are presented as lists, e.g., in Markush group format, each subgroup of the elements is also disclosed, and any element(s) may be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention may be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

Lengthy table referenced here
US20260009027A1-20260108-T00001
Please refer to the end of the specification for access instructions.

Lengthy table referenced here
US20260009027A1-20260108-T00002
Please refer to the end of the specification for access instructions.

Lengthy table referenced here
US20260009027A1-20260108-T00003
Please refer to the end of the specification for access instructions.

Lengthy table referenced here
US20260009027A1-20260108-T00004
Please refer to the end of the specification for access instructions.

Lengthy table referenced here
US20260009027A1-20260108-T00005
Please refer to the end of the specification for access instructions.

LENGTHY TABLES
The patent application contains a lengthy table section. A copy of the table is available in electronic form from the USPTO web site (https://seqdata.uspto.gov/docdetail?docId=US20260009027A1). An electronic copy of the table will also be available from the USPTO upon request and payment of the fee set forth in 37 CFR 1.19(b)(3).

1. A method for editing a DNA sequence encoding an endogenous tRNA at a target site, the method comprising contacting the DNA sequence at the target site with a prime editor and a pegRNA, wherein the prime editor installs one or more modifications in the DNA sequence at the target site, relative to the DNA sequence encoding the endogenous tRNA, thus converting the endogenous tRNA into a suppressor tRNA, wherein the pegRNA comprises a spacer sequence, a gRNA core, and an extension arm.

2. The method of claim 1, wherein the spacer sequence and extension arms are any sequences listed in Table 2, and/or wherein the DNA sequence is any sequence listed in Table 1.

3-5. (canceled)

6. The method of claim 1, wherein the target site in the DNA sequence encodes one or more domains of the endogenous tRNA, wherein the domain is selected from the group consisting of a D-arm domain, a variable arm domain, an acceptor stem domain, a T-arm domain, or an anticodon arm domain of the endogenous tRNA.

7-9. (canceled)

10. The method of claim 6, wherein the domain is an anticodon arm domain comprising an anticodon sequence of the tRNA.

11. (canceled)

12. The method of claim 1, wherein installing the one or more modifications comprises installing a single base nucleotide insertion in a variable arm domain, wherein the insertion replaces a cognate amino acid with a non-cognate amino acid.

13-14. (canceled)

15. The method of claim 1, wherein the one or more modifications comprises a C70U mutation in an acceptor stem domain.

16-17. (canceled)

18. The method of claim 1, wherein the one or more modifications comprises substituting the DNA sequence encoding an anticodon sequence with a nonsense suppressor anticodon sequence.

19. (canceled)

20. The method of claim 18, wherein the nonsense suppressor anticodon sequence is selected from the group consisting of 5′-UUA-3′, 5′-UCA-3′, and 5′-CUA-3′.

21-25. (canceled)

26. The method of claim 1, wherein the pegRNA directs the prime editor to install an edit at the target site located between positions +1 and +40 relative to a first editable base located 3′ of a pegRNA-directed nick.

27. (canceled)

28. The method of claim 1, wherein the extension arm comprises a DNA synthesis template and a primer binding site (PBS).

29. The method of claim 1, wherein the suppressor tRNA is used to treat a disease caused by a premature termination codon.

30-33. (canceled)

34. The method of claim 1, wherein installing one or more modifications in the DNA sequence at the target site comprises installing one or more PAM-disrupting mutations, MMR-evading mutations, or combinations thereof.

35. The method of claim 1, further comprising contacting the DNA sequence with a gRNA.

36. The method of claim 1, further comprising contacting the DNA sequence with a second pegRNA.

37-61. (canceled)

62. A prime editing guide RNA (pegRNA) for editing a DNA sequence encoding an endogenous tRNA by prime editing to produce a DNA sequence encoding a suppressor tRNA, wherein the pegRNA comprises a spacer sequence, a gRNA core, and an extension arm comprising a DNA synthesis template and a primer binding site (PBS), wherein the spacer sequence is configured to bind to a DNA sequence encoding an endogenous tRNA.

63-76. (canceled)

77. A complex comprising a prime editor and the pegRNA of claim 62.

78. A polynucleotide comprising a first nucleic acid sequence encoding a prime editor and a second nucleic acid sequence encoding the pegRNA of claim 62.

79-80. (canceled)

81. A cell comprising the complex of claim 77.

82-84. (canceled)

85. A pharmaceutical composition comprising the pegRNA of claim 62 or a polynucleotide encoding the pegRNA, a prime editor or a polynucleotide encoding the prime editor, and a pharmaceutical excipient.

86-114. (canceled)

115. A method for inserting a DNA sequence encoding a suppressor tRNA gene into a target site in a host genome using prime editing, the method comprising contacting the target site with (i) a prime editor, (ii) a pegRNA, (iii) a sgRNA, and (iv) a plasmid, wherein the prime editor comprises a fusion protein comprising a nucleic acid programmable DNA binding protein (napDNAbp), a DNA polymerase, and a recombinase.

116-172. (canceled)
